(19) United States
(12) Patent Application Publication
(10) Pub. No.: US 2025/0051433 A1
(43) Pub. Date: Feb. 13, 2025

(54) NOVEL ANTI-TSLP ANTIBODIES

(71) Applicant: INMAGENE PTE. LTD., Singapore (SG)

(72) Inventors: Run LEI, Shanghai (CN); Chongtian GUO, Shanghai (CN); Pengcheng FAN, Zhejiang, Hangzhou (CN); Zhihao XU, Zhejiang, Hangzhou (CN); Qiang SUN, Zhejiang, Hangzhou (CN); Jonathan Jian WANG

(21) Appl. No.: 18/722,607

(22) PCT Filed: Dec. 23, 2022
(86) PCT No.: PCT/CN2022/141670

§ 371 Date: Jun. 21, 2024

(30) Foreign Application Priority Data
Dec. 24, 2021 (WO) PCT/CN2021/141294
Nov. 25, 2022 (WO) PCT/CN2022/134293

(51) Int. Cl. C07K 16/24; A61K 39/00; C12N 5/0783; G01N 33/68

(52) U.S. Cl. CPC C07K 16/244; A61K 39/4611; A61K 39/4631; A61K 39/46444; C12N 5/0636; G01N 33/6869; C07K 2317/24; C07K 2317/732; C07K 2317/76; C07K 2317/92; C07K 2317/94; C12N 2510/00

(57) ABSTRACT

Provided herein are anti-TSLP antibodies or antigen-binding fragments thereof, isolated poly nucleotides encoding the same, pharmaceutical compositions comprising the same, and the uses thereof.

### FIELD OF THE INVENTION

The present disclosure generally relates to novel anti-TSLP antibodies.

### BACKGROUND

Thymic stromal lymphopoietin (TSLP) is a protein belonging to the cytokine family. It is known to play an important role in the maturation of T cell populations through activation of antigen presenting cells. TSLP is produced mainly by non-hematopoietic cells such as fibroblasts, epithelial cells and different types of stromal or stromal-like cells.

TSLP is a multifunctional cytokine that can exert biological functions through TSLP/IL-7Rα receptors on the surface of a variety of immune cells, including DC cells, CD4 and CD8+ T cells, B cells, ILC2, sensory neuron, mast cells, basophils, eosinophils and NKT cells. TSLP transduces signals through JAK/STAT (JAK kinase-signal transducer and activator of transcription) pathway. TSLP binds to TSLPR on the cell membrane and then binds to IL-7Rα to form a stable TSLP-TSLPR-IL7Rα complex. The intracellular segment of TSLPR receptor in the complex recruits and activates JAK2, which acts together with JAK1 recruited by IL7Rα to activate downstream signal molecules. Studies have shown that in human peripheral blood derived CD11c+DC cells, TSLP can activate STAT1, STAT3, STAT4, STAT5 and STAT6, among which STAT5 activation signal is the key to promote Th2 cell differentiation and secrete Th2 factor.

TSLP is known to control type 2 inflammation via activation of dendritic cells, mast cells, and ILC2s, which makes it closely related to the occurrence of type II inflammatory diseases.

Therefore, blocking of TSLP signaling is a potentially effective strategy for the treatment of asthma, allergic dermatitis and other diseases relating to TSLP signaling. However, TSLP-related diseases have high unmet medical needs. Therefore, needs remain for novel anti-TSLP antibodies.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one respect, the present disclosure provides an antibody or an antigen-binding fragment thereof capable of specifically binding to human TSLP, comprising heavy chain complementary determining region 1 (HCDR1), HCDR2 and HCDR3 contained within any one of the heavy chain variable region sequence of SEQ ID NO: 1 or 3, and/or light chain complementary determining region 1 (LCDR1), LCDR2 and LCDR3 contained within any one of the heavy chain variable region sequence of SEQ ID NO: 2 or 4.

In one respect, the present disclosure provides an antibody or an antigen-binding fragment thereof capable of specifically binding to human TSLP, comprising heavy chain complementary determining region 1 (HCDR1), HCDR2 and HCDR3, wherein (a) the HCDR1 comprises an amino acid sequence of SEQ ID NO: 11; the HCDR2 comprises an amino acid sequence of IFPGDGX₁T (SEQ ID NO: 59); and the HCDR3 comprises an amino acid sequence of ARX₂GX₃X₄X₅X₆X₇X₈YAMDY (SEQ ID NO: 60); wherein, X₁ is E or D; X₂ is G or S; X₃ is Y or F; X₄ is V or D; X₅ is N or Y; X₆ is none or G; X₇ is F or Y; X₈ is L or F; or (b) the HCDR1 comprises an amino acid sequence of SYWX₉N (SEQ ID NO: 61); the HCDR2 comprises an amino acid sequence of QIFPGDGX₁₀TX₁₁YNGX₁₂FKG (SEQ ID NO: 62); and the HCDR3 comprises an amino acid sequence of X₁₃GX₁₄X₁₅X₁₆X₁₇X₁₈X₁₉YAMDY (SEQ ID NO: 63); wherein, X₉ is M or I; X₁₀ is E or D; X₁₁ is N or T; X₁₂ is K or N; X₁₃ is G or S; X₁₄ is Y or F; X₁₅ is V or D; X₁₆ is N or Y; X₁₇ is none or G; X₁₈ is F or Y; X₁₉ is L or F.

In some embodiments, the antibody or an antigen-binding fragment thereof comprises light chain complementary determining region 1 (LCDR1), LCDR2 and LCDR3, wherein (a) the LCDR1 comprises an amino acid sequence of SEQ ID NO: 14; the LCDR2 comprises an amino acid sequence of SEQ ID NO: 15; and the LCDR3 comprises an amino acid sequence of SEQ ID NO: 16; or (b) the LCDR1 comprises an amino acid sequence of SEQ ID NO: 53; the LCDR2 comprises an amino acid sequence of GTSX₂₀LAS (SEQ ID NO: 64); and the LCDR3 comprises an amino acid sequence of SEQ ID NO: 16; wherein, X₂₀ is T or N.

In some embodiments, the antibody or an antigen-binding fragment thereof comprises a heavy chain variable region comprising HCDR1, HCDR2 and HCDR3, and/or a light chain variable region comprising LCDR1, LCDR2 and LCDR3, wherein (a) the HCDR1 comprises an amino acid sequence of SEQ ID NO: 11; the HCDR2 comprises an amino acid sequence of IFPGDGX₁T (SEQ ID NO: 59); and the HCDR3 comprises an amino acid sequence of ARX₂GX₃X₄X₅X₆X₇X₈YAMDY (SEQ ID NO: 60); the LCDR1 comprises an amino acid sequence of SEQ ID NO: 14, the LCDR2 comprises an amino acid sequence of SEQ ID NO: 15; and the LCDR3 comprises an amino acid sequence of SEQ ID NO: 16; wherein X₁ is E or D; X₂ is G or S; X₃ is Y or F; X₄ is V or D; X₅ is N or Y; X₆ is none or G; X₇ is F or Y; X₈ is L or F; or (b) the HCDR1 comprises an amino acid sequence of SYWX₉N (SEQ ID NO: 61); the HCDR2 comprises an amino acid sequence of QIFPGDGX₁₀TX₁₁YNGX₁₂FKG (SEQ ID NO: 62); and the HCDR3 comprises an amino acid sequence of X₁₃GX₁₄X₁₅X₁₆X₁₇X₁₈X₁₉YAMDY (SEQ ID NO: 63); the LCDR1 comprises an amino acid sequence of SEQ ID NO: 53; the LCDR2 comprises an amino acid sequence of GTSX₂₀LAS (SEQ ID NO: 64); and the LCDR3 comprises an amino acid sequence of SEQ ID NO: 16; wherein X₉ is M or I; X₁₀ is E or D; X₁₁ is N or T; X₁₂ is K or N; X₁₃ is G or S; X₁₄ is Y or F; X₁₅ is V or D; X₁₆ is N or Y; X₁₇ is none or G; X₁₈ is F or Y; X₁₉ is L or F; X₂₀ is T or N.

In some embodiments, the HCDR1 comprises an amino acid sequence of SEQ ID NO: 11, the HCDR2 comprises an amino acid sequence of SEQ ID NO: 12 or 25, the HCDR3 comprises an amino acid sequence of SEQ ID NO: 13 or 26, the LCDR1 comprises an amino acid sequence of SEQ ID NO: 14, the LCDR2 comprises an amino acid sequence of SEQ ID NO: 15, and the LCDR3 comprises an amino acid sequence of SEQ ID NO: 16.

In some embodiments, the HCDR1 comprises an amino acid sequence of SEQ ID NO: 50 or 55, the HCDR2 comprises an amino acid sequence of SEQ ID NO: 51 or 56, the HCDR3 comprises an amino acid sequence of SEQ ID NO: 52 or 57, the LCDR1 comprises an amino acid sequence of SEQ ID NO: 53, the LCDR2 comprises an amino acid sequence of SEQ ID NO: 54 or 58, and the LCDR3 comprises an amino acid sequence of SEQ ID NO: 16.

In some embodiments, (a) the HCDR1 comprises an amino acid sequence of SEQ ID NO: 11; the HCDR2 comprises an amino acid sequence of SEQ ID NO: 12; the HCDR3 comprises an amino acid sequence of SEQ ID NO: 13; the LCDR1 comprises an amino acid sequence of SEQ ID NO: 14; the LCDR2 comprises an amino acid sequence of SEQ ID NO: 15; and the LCDR3 comprises an amino acid sequence of SEQ ID NO: 16; (b) the HCDR1 comprises an amino acid sequence of SEQ ID NO: 11; the HCDR2 comprises an amino acid sequence of SEQ ID NO: 25; the HCDR3 comprises an amino acid sequence of SEQ ID NO: 26; the LCDR1 comprises an amino acid sequence of SEQ ID NO: 14; the LCDR2 comprises an amino acid sequence of SEQ ID NO: 15; and the LCDR3 comprises an amino acid sequence of SEQ ID NO: 16; (c) the HCDR1 comprises an amino acid sequence of SEQ ID NO: 50; the HCDR2 comprises an amino acid sequence of SEQ ID NO: 51; the HCDR3 comprises an amino acid sequence of SEQ ID NO: 52; the LCDR1 comprises an amino acid sequence of SEQ ID NO: 53; the LCDR2 comprises an amino acid sequence of SEQ ID NO: 54; and the LCDR3 comprises an amino acid sequence of SEQ ID NO: 16; (d) the HCDR1 comprises an amino acid sequence of SEQ ID NO: 55; the HCDR2 comprises an amino acid sequence of SEQ ID NO: 56; the HCDR3 comprises an amino acid sequence of SEQ ID NO: 57; the LCDR1 comprises an amino acid sequence of SEQ ID NO: 53; the LCDR2 comprises an amino acid sequence of SEQ ID NO: 58; and the LCDR3 comprises an amino acid sequence of SEQ ID NO: 16.

In some embodiments, the antibody or an antigen-binding fragment thereof provided herein further comprises one or more of heavy chain framework region 1 (HFR1), HFR2, HFR3 and HFR4, and/or one or more of light chain framework region 1 (LFR1), LFR2, LFR3 and LFR4, wherein the HFR1 comprises an amino acid sequence of SEQ ID NO: 17, SEQ ID NO: 27, or X₂₁VQLVQSGAEVKKPGX₂₂SX₂₃KX₂₄SCKX₂₅S (SEQ ID NO: 65), or a homologous sequence of at least 85% sequence identity thereof, the HFR2 comprises an amino acid sequence of SEQ ID NO: 18 or WVRQX₂₇PGX₂₈GLEWMG (SEQ ID NO: 66), or a homologous sequence of at least 85% sequence identity thereof, the HFR3 comprises an amino acid sequence of SEQ ID NO: 19, SEQ ID NO: 28, or X₃₁VTX₃₂X₃₃X₃₄DX₃₅SX₃₆STX₃₇YX₃₈X₃₉X₄₀SSLX₄₁X₄₂X₄₃DTAX₄₄YYC (SEQ ID NO: 67), or a homologous sequence of at least 85% sequence identity thereof, the HFR4 comprises an amino acid sequence of SEQ ID NO: 20 or SEQ ID NO: 35, or a homologous sequence of at least 85% sequence identity thereof, the LFR1 comprises an amino acid sequence of SEQ ID NO: 21, SEQ ID NO: 29, or SEQ ID NO: 36, or a homologous sequence of at least 85% sequence identity thereof, the LFR2 comprises an amino acid sequence of SEQ ID NO: 22, SEQ ID NO: 30, or WYQQKPGQSPRPWIX₄₅ (SEQ ID NO: 68), or a homologous sequence of at least 85% sequence identity thereof, the LFR3 comprises an amino acid sequence of SEQ ID NO: 23, SEQ ID NO: 31, or SEQ ID NO: 38, or a homologous sequence of at least 85% sequence identity thereof, and the LFR4 comprises an amino acid sequence of SEQ ID NO: 24 or SEQ ID NO: 39, or a homologous sequence of at least 85% sequence identity thereof, wherein, X₂₁ is E or Q; X₂₂ is E, S or A; X₂₃ is L or V; X₂₄ is I or V; X₂₅ is S or A; X₂₇ is M or A; X₂₈ is K or Q; X₃₁ is Q or R; X₃₂ is I or M; X₃₃ is S or T; X₃₄ is A or R; X₃₅ is K or T; X₃₆ is I or T; X₃₇ is A or V; X₃₈ is L or M; X₃₉ is Q or E; X₄₀ is W or L; X₄₁ is K or R; X₄₂ is A or S; X₄₃ is S or E; X₄₄ is M or V; X₄₅ is Y or F.

In some embodiments, the HFR1 comprises a sequence selected from the group consisting of SEQ ID NOs: 17, 27, 32, 40, 42, 47 and 87, the HFR2 comprises a sequence selected from the group consisting of SEQ ID NOs: 18, 33, 43 and 48, the HFR3 comprises the sequence selected from the group consisting of SEQ ID NOs: 19, 28, 34, 41, 44 and 49, the HFR4 comprises the sequence of SEQ ID NOs: 20 and 35, the LFR1 comprises the sequence of SEQ ID NOs: 21, 29 and 36, the LFR2 comprises the sequence selected from the group consisting of SEQ ID NOs: 22, 30, 37 and 45, the LFR3 comprises a sequence selected from the group consisting of SEQ ID NOs: 23, 31 and 38, and the LFR4 comprises a sequence selected from the group consisting of SEQ ID NOs: 24 and 39.

In some embodiments, (a) the HFR1 comprises an amino acid sequence of SEQ ID NO: 17, the HFR2 comprises an amino acid sequence of SEQ ID NO: 18, the HFR3 comprises an amino acid sequence of SEQ ID NO: 19, the HFR4 comprises an amino acid sequence of SEQ ID NO: 20, the LFR1 comprises an amino acid sequence of SEQ ID NO: 21, the LFR2 comprises an amino acid sequence of SEQ ID NO: 22, the LFR3 comprises an amino acid sequence of SEQ ID NO: 23, and the LFR4 comprises an amino acid sequence of SEQ ID NO: 24; (b) the HFR1 comprises an amino acid sequence of SEQ ID NO: 27, the HFR2 comprises an amino acid sequence of SEQ ID NO: 18, the HFR3 comprises an amino acid sequence of SEQ ID NO: 28, the HFR4 comprises an amino acid sequence of SEQ ID NO: 20, the LFR1 comprises an amino acid sequence of SEQ ID NO: 29, the LFR2 comprises an amino acid sequence of SEQ ID NO: 30, the LFR3 comprises an amino acid sequence of SEQ ID NO: 31, and the LFR4 comprises an amino acid sequence of SEQ ID NO: 24; (c) the HFR1 comprises an amino acid sequence of SEQ ID NO: 32, the HFR2 comprises an amino acid sequence of SEQ ID NO: 33, the HFR3 comprises an amino acid sequence of SEQ ID NO: 34, the HFR4 comprises an amino acid sequence of SEQ ID NO: 35, the LFR1 comprises an amino acid sequence of SEQ ID NO: 36, the LFR2 comprises an amino acid sequence of SEQ ID NO: 37, the LFR3 comprises an amino acid sequence of SEQ ID NO: 38, and the LFR4 comprises an amino acid sequence of SEQ ID NO: 39; (d) the HFR1 comprises an amino acid sequence of SEQ ID NO: 40, the HFR2 comprises an amino acid sequence of SEQ ID NO: 33, the HFR3 comprises an amino acid sequence of SEQ ID NO: 41, the HFR4 comprises an amino acid sequence of SEQ ID NO: 35, the LFR1 comprises an amino acid sequence of SEQ ID NO: 36, the LFR2 comprises an amino acid sequence of SEQ ID NO: 37, the LFR3 comprises an amino acid sequence of SEQ ID NO: 38, and the LFR4 comprises an amino acid sequence of SEQ ID NO: 39; (e) the HFR1 comprises an amino acid sequence of SEQ ID NO: 42, the HFR2 comprises an amino acid sequence of SEQ ID NO: 43, the HFR3 comprises an amino acid sequence of SEQ ID NO: 44, the HFR4 comprises an amino acid sequence of SEQ ID NO: 35, the LFR1 comprises an amino acid sequence of SEQ ID NO: 36, the LFR2 comprises an amino acid sequence of SEQ ID NO: 45, the LFR3 comprises an amino acid sequence of SEQ ID NO: 38, and the LFR4 comprises an amino acid sequence of SEQ ID NO: 39; (f) the HFR1 comprises an amino acid sequence of SEQ ID NO: 87, the HFR2 comprises an amino acid sequence of SEQ ID NO: 33, the HFR3 comprises an amino acid sequence of SEQ ID NO: 41, the HFR4 comprises an amino acid sequence of SEQ ID NO: 35, the LFR1 comprises an amino acid sequence of SEQ ID NO: 36, the LFR2 comprises an amino acid sequence of SEQ ID NO: 45, the LFR3 comprises an amino acid sequence of SEQ ID NO: 38, and the LFR3 comprises an amino acid sequence of SEQ ID NO: 39; (g) the HFR1 comprises an amino acid sequence of SEQ ID NO: 47, the HFR2 comprises an amino acid sequence of SEQ ID NO: 48, the HFR3 comprises an amino acid sequence of SEQ ID NO: 49, the HFR4 comprises an amino acid sequence of SEQ ID NO: 35, the LFR1 comprises an amino acid sequence of SEQ ID NO: 36, the LFR2 comprises an amino acid sequence of SEQ ID NO: 45, the LFR3 comprises an amino acid sequence of SEQ ID NO: 38, and the LFR4 comprises an amino acid sequence of SEQ ID NO: 39.

In some embodiments, the heavy chain variable region of the antibody or an antigen-binding fragment thereof provided herein comprises the sequence selected from the group consisting of SEQ ID NOs: 1, 3, 5, 7, 8, 10 and 86, and a homologous sequence thereof having at least 80% sequence identity yet retaining specific binding affinity to human TSLP.

In some embodiments, the light chain variable region of the antibody or an antigen-binding fragment thereof provided herein comprises the sequence selected from the group consisting of SEQ ID NOs: 2, 4, 6 and 9, and a homologous sequence thereof having at least 80% sequence identity yet retaining specific binding affinity to human TSLP.

In some embodiments, in the antibody or an antigen-binding fragment thereof provided herein, the heavy chain variable region comprises the sequence of SEQ ID NO: 1 and the light chain variable region comprises the sequence of SEQ ID NO: 2; or the heavy chain variable region comprises the sequence of SEQ ID NO: 3 and the light chain variable region comprises the sequence of SEQ ID NO: 4; or the heavy chain variable region comprises the sequence of SEQ ID NO: 5 and the light chain variable region comprises the sequence of SEQ ID NO: 6; or the heavy chain variable region comprises the sequence of SEQ ID NO: 7 and the light chain variable region comprises the sequence of SEQ ID NO: 6; or the heavy chain variable region comprises the sequence of SEQ ID NO: 86 and the light chain variable region comprises the sequence of SEQ ID NO: 9; or the heavy chain variable region comprises the sequence of SEQ ID NO: 8 and the light chain variable region comprises the sequence of SEQ ID NO: 9; or the heavy chain variable region comprises the sequence of SEQ ID NO: 10 and the light chain variable region comprises the sequence of SEQ ID NO: 9.

In some embodiments, the antibody or an antigen-binding fragment thereof provided herein further comprises one or more amino acid residue substitutions or modifications yet retains specific binding affinity to human TSLP. In some embodiments, at least one of the substitutions or modifications is in one or more of the CDR sequences, and/or in one or more of the non-CDR sequences of the heavy chain variable region or light chain variable region. In some embodiments, at least one of the substitutions is a conservative substitution.

In some embodiments, the antibody or an antigen-binding fragment thereof provided herein further comprises an Fc region, optionally an Fc region of human immunoglobulin (Ig), or optionally an Fc region of human IgG. In some embodiments, the Fc region is derived from human IgG1, IgG2, IgG3, IgG4, IgA1, IgA2 or IgM. In some embodiments, the Fc region derived from human IgG4 comprises mutations of S228P, F234A, L235A, M252Y, S254T, T256E or K447del (PAA-YTE). In some embodiments, the Fc region derived from human IgG1 comprises one or more mutations selected from the group consisting of L234A, L235A, M252Y, S254T, and T256E. In some embodiments, the Fc region derived from human IgG1 comprises mutations of L234A, L235A, M252Y, S254T, and T256E (LALA-YTE).

In some embodiments, the antibody or an antigen-binding fragment thereof provided herein further comprises a signal peptide at the N-terminal of the heavy chain variable region and/or a signal peptide at the N-terminal of the light chain variable region.

In some embodiments, the antibody or an antigen-binding fragment thereof provided herein is humanized. In some embodiments, the antibody or an antigen-binding fragment thereof provided herein is a monoclonal antibody, a bispecific antibody, a multi-specific antibody, a recombinant antibody, a chimeric antibody, a labeled antibody, a bivalent antibody, an anti-idiotypic antibody or a fusion protein.

In some embodiments, the antibody or an antigen-binding fragment thereof provided herein is a diabody, a Fab, a Fab′, a F(ab′)₂, an Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In some embodiments, the antibody or an antigen-binding fragment thereof provided herein has one or more properties selected from the group consisting of: a) having the ability to specifically bind to human TSLP; b) having the ability to block the binding between TSLP with TSLPR; c) having the ability to block the binding between TSLP with cells expressing TSLPR and IL7R; d) having the ability to inhibit the TSLP dependent proliferation of BaF3 cells; e) having the ability to inhibit the TSLP dependent TARC secretion from PBMC; or f) having the ability to inhibit the TSLP dependent STAT5 activation in cells expressing TSLPR and IL7R.

In some embodiments, the antibody or antigen-binding fragment thereof does not specifically bind to TSLP of monkey, rat or mouse.

In some embodiments, the antibody or antigen-binding fragment thereof specifically binds to long-form human TSLP and does not bind to short-form human TSLP; wherein, the long-form human TSLP has an amino acid sequence as shown in SEQ ID NO: 73, and the short-form human TSLP has an amino sequence as shown in SEQ ID NO: 74.

In some embodiments, the antibody or antigen-binding fragment thereof has the ability to inhibit the human TSLP dependent proliferation of human TSLPR-expressing cells. In some embodiments, the cells further express human IL7R.

In some embodiments, the antibody or an antigen-binding fragment thereof binds to a different epitope from what a reference antibody binds, wherein the amino acid sequence of the heavy chain of reference antibody is shown as SEQ ID NO: 77, and the amino acid sequence of the light chain of reference antibody is shown as SEQ ID NO: 78.

In some embodiments, the antibody or an antigen-binding fragment thereof is linked to one or more conjugate moieties. In some embodiments, the conjugate moiety comprises an agent for detection or isolation, such as a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, or other anticancer drugs.

In another aspect, the present disclosure provides an isolated polynucleotide encoding the antibody or an antigen-binding fragment thereof disclosed herein. In another aspect, the present disclosure provides a vector comprising the isolated polynucleotide disclosed herein. In another aspect, the present disclosure provides a host cell comprising the vector disclosed herein.

In another aspect, the present disclosure provides a pharmaceutical composition, comprising: (i) the antibody or an antigen-binding fragment thereof or the polynucleotide encoding the antibody or antigen-binding fragment thereof disclosed herein; and (ii) one or more pharmaceutically acceptable carriers, diluent, buffer or excipient.

In some embodiments, the pharmaceutical composition further comprises an additional therapeutic agent. In some embodiments, the additional therapeutic agent is an agent for treating an inflammatory disease, an autoimmune disease, and a cancer. In some embodiments, the additional therapeutic agent is an agent targeting IL-33, IL-25, IL-4, IL-5, IL-4R, or IL-13.

In another aspect, the present disclosure provides a method of expressing the antibody or an antigen-binding fragment thereof disclosed herein, comprising culturing the host cell disclosed herein under the condition at which the vector disclosed herein is expressed.

In another aspect, the present disclosure provides a method of treating, preventing or alleviating a TSLP-related disease or disorder in a subject, comprising administering to the subject a therapeutically effective amount of the antibody or an antigen-binding fragment thereof capable of binding to long-form human TSLP having an amino acid sequence as shown in SEQ ID NO: 73, and a digested form of TSLP.

In another aspect, the present disclosure provides a method of treating, preventing or alleviating a TSLP-related disease or disorder in a subject, comprising administering to the subject a therapeutically effective amount of the antibody or an antigen-binding fragment thereof, or the polynucleotide encoding the antibody or antigen-binding fragment thereof, and/or the pharmaceutical composition disclosed herein. In some embodiments, the disease or disorder is associated with dysregulation of TSLPR mediated signaling compared to the control level. In some embodiments, the dysregulation of TSLP mediated signaling includes dysregulation of STAT5 activation by phosphorylation. In some embodiments, the dysregulation of TSLP mediated signaling includes dysregulation of TSLPR-expressing cell proliferation.

In some embodiments, the disease or disorder is selected from the group consisting of an inflammatory disease, an autoimmune disease, and a cancer.

In some embodiments, the disease or disorder is selected from the group consisting of: asthma (including severe asthma), idiopathic pulmonary fibrosis, atopic dermatitis (AD), allergic conjunctivitis, allergic rhinitis (AR), Netherton syndrome (NS), eosinophilic esophagitis (EoE), food allergy, allergic diarrhoea, eosinophilic gastroenteritis, allergic bronchopulmonary aspergillosis (ABPA), allergic fungal sinusitis, chronic pruritus, cancer, rheumatoid arthritis, Chronic obstructive pulmonary disease COPD, systemic sclerosis, multiple sclerosis, keloids, ulcerative colitis, chronic rhinosinusitis (CRS), polyposis, chronic eosinophilic pneumonia, eosinophilic bronchitis, allergic bronchopulmonary aspergillosis, coeliac disease, eosinophilic gastroenteritis, ChurgStrauss syndrome, eosinophilic myalgia syndrome, hypereosinophilic syndrome, eosinophilic granulomatosis with polyangiitis, eosinophilic esophagitis, inflammatory bowel disease, fibrotic disorder, inflammatory bowel disease Hodgkin's lymphoma, systemic lupus erythematosus.

In some embodiments, the cancer is selected from the group consisting of: breast cancer, pancreas cancer, colon cancer, lung cancer, ovarian cancer, prostate cancer, and B-cell acute lymphoblastic leukemia.

In some embodiments, wherein the fibrotic disorder is selected from the group consisting of: systemic and local scleroderma, keloids and hypertrophic scars, interstitial lung disease (ILD), idiopathic pulmonary fibrosis (IPF), liver fibrosis resulting from chronic hepatitis B or C infection, radiation-induced fibrosis, and fibrosis arising from wound healing, atherosclerosis, restinosis, pulmonary inflammation and fibrosis, liver cirrhosis, kidney disease, heart disease resulting from scar tissue, and eye diseases such as macular degeneration, and retinal and vitreal retinopathy, fibrosis resulting from chemotherapeutic drugs, and injuries and burns.

In some embodiments, the disease or disorder is selected from the group consisting of asthma, Polyp nasal Sinusitis, COPD, urticaria, EoE, and atopic dermatitis.

In another aspect, the present disclosure provides a method of detecting the presence or amount of TSLP in a sample, comprising contacting the sample with the antibody or an antigen-binding fragment thereof disclosed herein, and determining the presence or the amount of TSLP in the sample. In some embodiments, the method further comprises a step of determining whether TSLP is over-expressed in the cells in the sample.

In another aspect, the present disclosure provides use of the antibody or an antigen-binding fragment thereof, the pharmaceutical composition, and/or the polynucleotide encoding the antibody or antigen-binding fragment thereof disclosed herein in the manufacture of a medicament for treating, preventing or alleviating a disease or disorder that is related to TSLP.

In another aspect, the present disclosure provides a chimeric antigen receptor (CAR) comprising an antigen binding domain, a transmembrane domain, and a TCR signaling domain, wherein the antigen binding domain specifically binds to TSLP and comprises an antigen binding fragment disclosed herein. In another aspect, the present disclosure provides a nucleic acid sequence encoding the chimeric antigen receptor (CAR) disclosed herein. In another aspect, the present disclosure provides a cell comprising the nucleic acid sequence disclosed herein. In another aspect, the present disclosure provides a vector comprising the nucleic acid sequence disclosed herein. In another aspect, the present disclosure provides a method for stimulating a T cell-mediated immune response to a TSLP-enriched environment or tissue in a mammal, the method comprising administering to the mammal an effective amount of a cell genetically modified to express the CAR disclosed herein.

In another aspect, the present disclosure provides a method of treating a mammal having a disease or disorder that is responsive to TSLP inhibition, comprising administering to the mammal an effective amount of a cell disclosed herein, thereby treating the mammal. In some embodiments, the cell is an autologous T cell. In some embodiments, the mammal is a human subject. In some embodiments, the mammal is identified as having a TSLP positive cell, or a cell with TSLP signaling up-regulated.

### BRIEF DESCRIPTION OF THE DRAWINGS

FIG. 1 shows the binding of antibody Chi39H11 and BMK to TSLP as measured by ELISA.

FIG. 2 shows the binding of antibodies Chi39H11, BMK and the control to fusion protein of human long form TSLP expressed on cell membrane as measured by FACS.

FIG. 3 shows the activity of antibodies Chi39H11, BMK and the control to block binding of human long form TSLP to TSLPR-expressing cells.

FIG. 4a and FIG. 4b show the cell binding of antibodies Chi39H11, 39H11Z07 (FIG. 4a), 39H11Z11 (FIG. 4b), BMK and the control to human long form TSLP expressed on cell membrane.

FIG. 5a and FIG. 5b show the activity of antibodies Chi39H11, 39H11Z07 (FIG. 5a), 39H11Z11 (FIG. 5b), BMK and the control to block binding of human long form TSLP to TSLPR-expressing cells.

FIG. 6 shows the activity of antibodies Chi39H11, 39H11Z07, 39H11Z11, BMK and the control to inhibit recombinant long form TSLP dependent BaF3 proliferation.

FIG. 7 shows the cell binding of antibodies Chi35G12, BMK and the control to human long form TSLP expressed on cell membrane.

FIG. 8 shows the activity of antibodies Chi35G12, BMK and the control to inhibit recombinant long form TSLP dependent BaF3 proliferation.

FIG. 9 shows the cell binding of antibodies Chi35G12, 35G12Z01, 35G12Z02, 35G12Z03, BMK and the control to human long form TSLP expressed on cell membrane.

FIG. 10 shows the activity of antibodies Chi35G12, 35G12Z01, 35G12Z02, 35G12Z03, BMK and the control to block binding of human long form TSLP to TSLPR-expressing cells.

FIG. 11 shows the activity of antibodies Chi35G12, 35G12Z01, 35G12Z02, 35G12Z03, BMK and the control to inhibit recombinant TSLP dependent BaF3 proliferation.

FIG. 12 shows the protein binding of antibodies mAb35G12, mAb39H11, BMK and the control to TSLP.

FIG. 13a to FIG. 13d show the protein binding of antibodies mAb35G12, mAb39H11, BMK and the control to human TSLP protein (FIG. 13a), Cyno TSLP (FIG. 13b), mouse TSLP (FIG. 13c), and rat TSLP (FIG. 13d).

FIG. 14 shows the results of epitope analysis of antibodies 39H11, 35G12 and BMK.

FIG. 15 shows the activity of antibodies mAb35G12, mAb39H11, BMK and the control to block binding of human long form TSLP to TSLPR-expressing cells.

FIG. 16 shows the cell binding of antibodies mAb35G12, mAb39H11, BMK and the control to human short form TSLP expressed on cell membrane.

FIG. 17 shows that TSLP protein 52Ha (i.e. long form mutant TSLP), 52Hb (long form native TSLP) and 52Hb-d (i.e. digested products of 52Hb) was immunoblotted with anti-his-HRP.

FIGS. 18a and 18b show the protein binding of antibodies 35G12Z03-IgG2, 39H11Z07-IgG2 and BMK to digested TSLP.

FIG. 19 shows the inhibition of TSLP dependent STAT5 activation by antibodies mAb35G12, mAb35G12_IgG1, mAb39H11, BMK and the control.

FIG. 20 shows the inhibition of natural TSLP dependent TARC secretion by antibodies mAb35G12, mAb39H11, BMK and the control.

FIG. 21 shows the inhibition of TSLP dependent BaF3 proliferation by antibodies mAb35G12, mAb35G12_IgG1, mAb39H11, BMK and the control.

FIG. 22 shows the inhibition of natural TSLP dependent BaF3 proliferation by antibodies mAb35G12, mAb39H11, BMK and the control.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273 (4), 927 (1997); Chothia, C. et al., J Mol Biol. December 5; 186 (3): 651-63 (1985); Chothia, C. and Lesk, A. M., J. Mol. Biol., 196,901 (1987); Chothia, C. et al., Nature. December 21-28; 342 (6252): 877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27:55-77 (2003); Marie-Paule Lefranc et al., Immunome Research, 1 (3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

“Fab” with regard to an antibody refers to that portion of the antibody consisting of a single light chain (both variable and constant regions) bound to the variable region and first constant region of a single heavy chain by a disulfide bond.

“Fab” refers to a Fab fragment that includes a portion of the hinge region.

“F(ab′)₂” refers to a dimer of Fab′.

“Fc” with regard to an antibody (e.g., of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85: 5879 (1988)).

“Single-chain Fv-Fc antibody” or “scFv-Fc” refers to an engineered antibody consisting of an scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231 (1-2): 25-38 (1999); Muyldermans S., J Biotechnol. June; 74 (4): 277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363 (6428): 446-8 (1993); Nguyen V K. et al. Immunogenetics. April; 54 (1): 39-47 (2002); Nguyen V K. et al. Immunology. May; 109 (1): 93-101 (2003)). The variable domain of a heavy chain antibody (VHH domain) represents the smallest known antigen-binding unit generated by adaptive immune responses (Koch-Nolte F. et al., FASEB J. November; 21 (13): 3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a V_{H} domain connected to a V_{L} domain in the same polypeptide chain (V_{H}-V_{L} Or V_{L}-V_{H}) (see, e.g., Holliger P. et al., Proc Natl Acad Sci USA. July 15; 90 (14): 6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or an antigen-binding fragment having multiple antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

In certain embodiments, an “scFv dimer” is a bivalent diabody or bispecific scFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate, and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g., a long flexible linker) and bound to two V_{L} moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human.

The term “affinity” as used herein refers to the strength of non-covalent interaction between an immunoglobulin molecule (i.e., antibody) or fragment thereof and an antigen.

The term “specific binding” or “specifically binds” as used herein refers to a non-random binding reaction between two molecules, such as for example between an antibody and an antigen. Specific binding can be characterized in binding affinity, for example, represented by K_{D} value, i.e., the ratio of dissociation rate to association rate (k_{off}/kₒₙ) when the binding between the antigen and antigen-binding molecule reaches equilibrium. K_{D} may be determined by using any conventional method known in the art, including but are not limited to, surface plasmon resonance method, microscale thermophoresis method, HPLC-MS method and flow cytometry (such as FACS) method. A K_{D} value of ≤10⁻⁶ M (e.g. ≤5×10⁻⁷M, ≤2×10⁻⁷M, ≤10⁻⁷M, ≤5×10⁻⁸ M, ≤2×10⁻⁸ M, ≤10⁻⁸ M, ≤5×10⁻⁹ M, ≤4×10⁻⁹M, ≤3×10⁻⁹M, ≤2×10⁻⁹ M, or ≤10⁻⁹ M) can indicate specific binding between an antibody or antigen binding fragments thereof and TSLP (e.g. human TSLP).

The ability to “compete for binding to human TSLP” as used herein refers to the ability of a first antibody or antigen-binding fragment to inhibit the binding interaction between human TSLP and a second anti-TSLP antibody to any detectable degree. In certain embodiments, an antibody or antigen-binding fragment that compete for binding to human TSLP inhibits the binding interaction between human TSLP and a second anti-TSLP antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e., including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Name of Amino Acid | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among amino acid residues with neutral hydrophilic side chains (e.g., Cys, Ser, Thr, Asn and Gln), among amino acid residues with acidic side chains (e.g., Asp, Glu), among amino acid residues with basic side chains (e.g., His, Lys, and Arg), or among amino acid residues with aromatic side chains (e.g., Trp, Tyr, and Phe). As known in the art, conservative substitution usually does not cause significant change in the protein conformational structure, and therefore could retain the biological activity of a protein.

The term “homologous” as used herein refers to nucleic acid sequences (or its complementary strand) or amino acid sequences that have sequence identity of at least 60% (e.g., at least 65%, 70%, 75%, 80%, 85%, 88%, 90%, 91%, 92%, 93%, 94%, 95%, 96%, 97%, 98%, 99%) to another sequence when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23 (21): 2947-8 (2007)), and ALIGN or Megalign (DNASTAR) software. A person skilled in the art may use the default parameters provided by the tool or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or an antigen-binding fragment thereof, at least one promoter (e.g., SV40, CMV, EF-1α) operably linked to the nucleic acid sequence, and at least one selection marker.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis” “diagnoses” or “diagnosing” refers to the identification of a pathological state, disease or condition, such as identification of a TSLP related disease, or refer to identification of a subject with a TSLP related disease who may benefit from a particular treatment regimen. Wherein, the TSLP related disease refers to a disease or disorder that is responsive to TSLP inhibition. In some embodiments, diagnosis contains the identification of abnormal amount or activity of TSLP. In some embodiments, diagnosis refers to the identification of an inflammatory disease, an autoimmune disease, or a cancer in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from heart, liver, spleen, lung, kidney, skin or blood vessels of the subject.

“TSLP” as used herein, refers to thymic stromal lymphopoietin, which is a protein belonging to the cytokine family and is known to be involved in type 2 inflammation via activation of dendritic cells, mast cells and ILC2s. TSLP signals through a heterodimeric receptor complex composed of the thymic stromal lymphopoietin receptor CRLF2 and the IL-7R alpha chain. After binding of TSLP to the heterodimeric receptor complex, STAT5 phosphorylation is induced, resulting in the expression of upstream transcription factors.

TSLP binds to a heterodimeric receptor consisting of the IL-7 receptor α-chain (IL-7Rα) and the TSLPR chain, which is closely related to the common receptor γ chain (γc), in order to exert its biological activity on a broad range of cell types. TSLPR alone has low affinity for TSLP but binding of TSLPR to IL-7Rα creates a high affinity binding site for TSLP and triggers signaling. In some embodiments, a fusion protein of human IL7Rα and human thymic stromal lymphopoietin receptor (TSLPR) can be used to trigger TSLP signaling. In some embodiments, the fusion protein comprises an amino acid sequence as set forth in SEQ ID NO: 70.

Amino acid sequence of the huIL7Ra-huTSLPR fusion protein (SEQ ID NO: 70):

MGWSCIILFLVATATGVHSESGYAQNGDLEDAELDDYSFSCYSQLEV
NGSQHSLTCAFEDPDVNITNLEFEICGALVEVKCLNFRKLQEIYFIE
TKKFLLIGKSNICVKVGEKSLTCKKIDLTTIVKPEAPFDLSVVYREG
ANDFVVTFNTSHLQKKYVKVLMHDVAYRQEKDENKWTHVNLSSTKLT
LLQRKLQPAAMYEIKVRSIPDHYFKGFWSEWSPSYYFRTPEINNSSG
EMDGGSGGSGGSGGSGGSGGSGGSGGSGGSGGSGGSGGSGGSGGSGG
SGGSGGSGGSGGSGGSQGGAAEGVQIQIIYFNLETVQVTWNASKYSR
TNLTFHYRFNGDEAYDQCTNYLLQEGHTSGCLLDAEQRDDILYFSIR
NGTHPVFTASRWMVYYLKPSSPKHVRFSWHQDAVTVTCSDLSYGDLL
YEVQYRSPFDTEWQSKQENTCNVTIEGLDAEKCYSFWVRVKAMEDVY
GPDTYPSDWSEVTCWQRGEIRDACAETPTPPKPKLSK

Human IL-7Rα has an amino acid sequence as set forth in SEQ ID NO: 71.

Amino acid sequence of the human IL-7Rα protein (SEQ ID NO: 71):

MTILGTTFGMVFSLLQVVSGESGYAQNGDLEDAELDDYSFSCYS
QLEVNGSQHSLTCAFEDPDVNITNLEFEICGALVEVKCLNFRKLQEIYF
IETKKFLLIGKSNICVKVGEKSLTCKKIDLTTIVKPEAPFDLSVVYREG
ANDFVVTFNTSHLQKKYVKVLMHDVAYRQEKDENKWTHVNLSSTKLTLL
QRKLQPAAMYEIKVRSIPDHYFKGFWSEWSPSYYFRTPEINNSSGEMDP
ILLTISILSFFSVALLVILACVLWKKRIKPIVWPSLPDHKKTLEHLCKK
PRKNLNVSFNPESFLDCQIHRVDDIQARDEVEGFLQDTFPQQLEESEKQ
RLGGDVQSPNCPSEDVVITPESFGRDSSLTCLAGNVSACDAPILSSSRS
LDCRESGKNGPHVYQDLLLSLGTTNSTLPPPFSLQSGILTLNPVAQGQP
ILTSLGSNQEEAYVTMSSFYQNQ

Human TSLPR comprises an amino acid sequence as set forth in SEQ ID NO: 72.

Amino acid sequence of the human TSLPR (SEQ ID NO: 72):

MGRLVLLWGAAVFLLGGWMALGQGGAAEGVQIQIIYFNLETVQ
VTWNASKYSRTNLTFHYRFNGDEAYDQCTNYLLQEGHTSGCLLDAEQRD
DILYFSIRNGTHPVFTASRWMVYYLKPSSPKHVRFSWHQDAVTVTCSDL
SYGDLLYEVQYRSPFDTEWQSKQENTCNVTIEGLDAEKCYSFWVRVKAM
EDVYGPDTYPSDWSEVTCWQRGEIRDACAETPTPPKPKLSKFILISSLA
ILLMVSLLLLSLWKLWRVKKFLIPSVPDPKSIFPGLFEIHQGNFQEWIT
DTQNVAHLHKMAGAEQESGPEEPLVVQLAKTEAESPRMLDPQTEEKEAS
GGSLQLPHQPLQGGDVVTIGGFTFVMNDRSYVAL

It has been described in mice that there exist two isoforms of TSLP, short and long isoforms, but the functional consequences of this variation are unclear. In humans, the main isoform expressed during steady state conditions is the short form of TSLP, whereas the long form of TSLP is upregulated in inflammatory conditions.

In some embodiments, the antibody or antigen-binding fragment disclosed herein binds to long-form TSLP but does not bind to short-form TSLP. In some embodiments, the TSLP to which the antibody or antigen-binding fragment disclosed herein specifically binds is the digested form of TSLP. In some embodiments, the antibody or antigen-binding fragment disclosed herein binds to the digested form of TSLP more potently, compared with existing antibodies (such as, AMG 157).

In some embodiments, the long-form TSLP comprises amino acid residues from position 29 to 159 of the full-length TSLP, while the short-form TSLP comprises amino acid residues from position 97 to 159 of the full-length TSLP. In some embodiments, the amino acid sequence of full-length TSLP is shown in the sequence with an NCBI accession number of NP_149024.1 (SD.hTSLP, i.e., SEQ ID NO: 85).

(SEQ ID NO: 85)
MFPFALLYVLSVSFRKIFILQLVGLVLTYDFTNCDFEKIKAAYLSTI
SKDLITYMSGTKSTEFNNTVSCSNRPHCLTEIQSLTFNPTAGCASLA
KEMFAMKTKAALAIWCPGYSETQINATQAMKKRRKRKVTTNKCLEQV
SQLQGLWRRFNRPLLKQQ

Native full-length or long-form human TSLP protein is prone to enzymatic cleavage, e.g. by PCSK3 enzyme. The PCSK3 enzyme can specifically target the positions 127 to 130 of the long-form TSLP protein and specifically truncates the protein at the site between positions 130 and 130, wherein the amino acid numbering is according to the sequence of full-length human TSLP protein. To prevent from such enzymatic cleavage and stabilize TSLP in its long form, certain mutations can be introduced to the enzymatic cleavage site (such as R127 and R130). Accordingly, in the present disclosure, the reference to long-form TSLP should be understood to encompass both the native long form TSLP and a mutated and stabilized long form TSLP, for example, a mutated TSLP with R127A and R130A mutations (e.g. SEQ ID NO: 73), or with R127A and R130S mutations (e.g. the TSLP sequence in SEQ ID NO: 80). In some embodiments, the long-form TSLP comprise an amino acid sequence as set forth in SEQ ID NO: 73. In some embodiments, the short-form TSLP comprise an amino acid sequence as set forth in SEQ ID NO: 74.

Amino acid sequence of long-form TSLP (SEQ ID NO: 73):

YDFTNCDFEKIKAAYLSTISKDLITYMSGTKSTEFNNTVSCSNRPHC
LTEIQSLTFNPTAGCASLAKEMFAMKTKAALAIWCPGYSETQINATQ
AMKKARKAKVTTNKCLEQVSQLQGLWRRFNRPLLKQQ

Amino acid sequence of short-form TSLP (SEQ ID NO: 74):

MFAMKTKAALAIWCPGYSETQINATQAMKKARKAKVTTNKCL
EQVSQLQGLWRRFNRPLLKQQ

The term “digested form of TSLP” as used herein refers to enzymatic digested product of a long form TSLP. In some embodiments, a digested form of TSLP can be obtained by the digestion of wild type long-form human TSLP protein by PCSK3 enzyme. In some embodiments, the PCSK3 enzyme specifically targets the positions 127 to 130 of the long-form TSLP protein and specifically truncates the protein at the site between positions 130 and 130, wherein the amino acid numbering is according to the sequence of full-length human TSLP protein. In some embodiments, the digested form of TSLP comprises a TSLP fragment comprising amino acid residues from position 29 to 124 of the full-length TSLP, and/or a TSLP fragment comprising amino acid residues from position 131 to 159 of the full-length TSLP. In some embodiments, the TSLP fragments further comprise a His-tag at the C-terminal region. In some embodiments, the digested form of TSLP is presented as a hetero-dimerized form, wherein the two heterologous monomers are linked via disulfide bonds. It is known in the art that expression level of the digested form of TSLP, especially presented as a hetero-dimerized form, is significantly higher in the inflammatory physiological conditions, compared with that in normal physiological conditions.

The term “anti-TSLP antibody” refers to an antibody that is capable of specifically binding to long form TSLP (e.g., human long form TSLP). In certain embodiments, the anti-TSLP antibody is further capable of specifically binding to a digested product of TSLP. The term “anti-human TSLP antibody” refers to an antibody that is capable of specifically binding to human TSLP or digested form of human TSLP.

A “TSLP-related” disease, disorder or condition refers to any disease or condition caused by, exacerbated by, or otherwise linked to increased or decreased expression or activities of long form TSLP. In some embodiments, the TSLP-related disease or disorder is a disease or disorder that is responsive to long form TSLP inhibition.

In some embodiments, the TSLP related disease, disorder or condition is associated with dysregulation of long form TSLP mediated signaling compared to the control level. In some embodiments, the dysregulation of long form TSLP mediated signaling includes dysregulation of STAT5 activation by phosphorylation. In some embodiments, the dysregulation of long form TSLP mediated signaling includes dysregulation of TSLPR-expressing cell proliferation. In some embodiments, the control level is the level in a healthy individual.

In some embodiments, the TSLP related disease, disorder or condition is an immune-related disorder, such as, for example, an autoimmune disease. In some embodiments, the TSLP related disease, disorder or condition is a disorder related to excessive cell proliferation, such as, for example, cancer. In certain embodiments, the TSLP related disease or condition is characterized in expressing or over-expressing of TSLP gene. In certain embodiments, the TSLP related disease or condition is characterized in over-expression of TSLP and/or dysregulation of TSLP mediated signaling.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “TSLP-positive cell” as used herein refer to a cell which shows an abnormal expression level of TSLP relative to a control cell. The abnormal expression level can be up-regulated or down-regulated relative to the level of the control cell and can be associated with dysregulation of TSLP mediated signaling. The control cell can be a normal or healthy counterpart cell, which may or may not express TSLP. In case the control cell expresses TSLP, the abnormal expression level of the TSLP-positive cell can be up-regulated or down-regulated. In case the control cell does not express TSLP, the abnormal expression level of the TSLP-positive cell can be up-regulated.

### Anti-TSLP Antibodies

The present disclosure provides anti-TSLP antibodies and antigen-binding fragments thereof. The anti-TSLP antibodies and antigen-binding fragments provided herein are capable of specific binding to long form TSLP.

In certain embodiments, the antibodies and the antigen-binding fragments thereof provided herein specifically bind to human TSLP (i.e., long-form TSLP) at an K_{D} value of no more than 10⁻⁷ M, no more than 8× 10⁻⁸ M, no more than 5×10⁻⁸ M, no more than 2×10⁻⁸ M, no more than 8×10⁻⁹ M, no more than 5×10⁻⁹ M, no more than 2×10⁻⁹ M, no more than 10⁻⁹ M, no more than 8×10⁻¹⁰ M, no more than 7×10⁻¹⁰ M, or no more than 6×10⁻¹⁰ M by Surface Plasmon Resonance (SPR) assay, see, for example, Murphy, M. et al., Current protocols in protein science, Chapter 19, unit 19.14, 2006.

Binding of the antibodies or the antigen-binding fragments thereof provided herein to human TSLP can also be represented by “half maximal effective concentration” (EC₅₀) value, which refers to the concentration of an antibody where 50% of its maximal binding is observed. The EC₅₀ value can be measured by binding assays known in the art, for example, direct or indirect binding assay such as enzyme-linked immunosorbent assay (ELISA), flow cytometry assay, and other binding assays.

In certain embodiments, the antibodies and antigen-binding fragments thereof provided herein exhibit no detectable binding to cynomolgus, rat or mouse TSLP or exhibits a binding to cynomolgus, rat or mouse TSLP at a level comparable to that of a negative control antibody under equivalent assay conditions. In addition, a negative control antibody can be any antibody that is known not to bind to cynomolgus, rat or mouse TSLP.

In certain embodiments, the antibodies and antigen-binding fragments thereof provided herein is capable of blocking TSLP signaling induced by a TSLP agonist (such as a fusion protein comprising TSLP and IL-7Rα), as measured by TSLP reporter assay.

In certain embodiments, the TSLP reporter assay is an assay measuring the level of STAT5 activation. In certain embodiments, the TSLP reporter assay is an assay measuring the secretion of TARC. In certain embodiments, the TSLP reporter assay is an assay measuring the TSLP dependent BaF3 proliferation.

In certain embodiments, the antibodies or the antigen-binding fragments thereof provided herein specifically bind to the digested form of TSLP, with a binding affinity higher than that of a control antibody (such as AMG157). In some embodiments, the binding affinity of the antibodies or the antigen-binding fragments thereof provided herein to the digested form of TSLP is at least 100% higher (e.g. at least 150% higher, 200% higher, 300% higher, 400% higher, 500% higher, 600% higher, 700% higher, 800% higher, 900% higher etc.) than that of AMG157. The binding affinity can be measured by the conventional means well known by those skilled in the art, such as western blotting, ELISA binding assay, fluorescence activated cell sorting (FACS) assay, biolayer interferometry (BLI) assay, surface plasmon resonance (SPR) assay, etc.

### Illustrative Anti-TSLP Antibodies

In certain embodiments, the present disclosure provides anti-TSLP antibodies (e.g., anti-human TSLP antibodies) and antigen-binding fragments thereof comprising one or more (e.g., 1, 2, or 3) HCDRs comprising the sequences selected from the group consisting of SEQ ID NO: 11, IFPGDGX₁T (SEQ ID NO: 59), and ARX₂GX₃X₄X₅X₆X₇X₈YAMDY (SEQ ID NO: 60); wherein, X₁ is E or D; X₂ is G or S; X₃ is Y or F; X₄ is V or D; X₅ is N or Y; X₆ is none or G; X₇ is F or Y; X₈ is L or F; or the group consisting of SYWX₉N (SEQ ID NO: 61), QIFPGDGX₁₀TX₁₁YNGX₁₂FKG (SEQ ID NO: 62), and X₁₃GX₁₄X₁₅X₁₆X₁₇X₁₈X₁₉YAMDY (SEQ ID NO: 63); wherein, X₉ is M or I; X₁₀ is E or D; X₁₁ is N or T; X₁₂ is K or N; X₁₃ is G or S; X₁₄ is Y or F; X₁₅ is V or D; X₁₆ is N or Y; X₁₇ is none or G; X₁₈ is F or Y; X₁₉ is L or F. In certain embodiments, the present disclosure further encompasses antibodies and antigen binding fragments thereof having no more than one, two or three amino acid residue substitutions to any of the sequences herein.

In certain embodiments, the present disclosure provides anti-TSLP antibodies (e.g., anti-human TSLP antibodies) and antigen-binding fragments thereof comprising one or more (e.g., 1, 2, or 3) LCDRs comprising the sequences selected from the group consisting of SEQ ID NO: 14, SEQ ID NO: 15, and SEQ ID NO: 16; or the group consisting of SEQ ID NO: 53, GTSX₂₀LAS (SEQ ID NO: 64), and SEQ ID NO: 16; wherein, X₂₀ is T or N. In certain embodiments, the present disclosure further encompasses antibodies and antigen binding fragments thereof having no more than one, two or three amino acid residue substitutions to any of the sequences herein.

Antibody “39H11” as used herein refers to a monoclonal antibody comprising a heavy chain variable region having the sequence of SEQ ID NO: 1, and a light chain variable region having the sequence of SEQ ID NO: 2.

Antibody “35G12” as used herein refers to a monoclonal antibody comprising a heavy chain variable region having the sequence of SEQ ID NO: 3, and a light chain variable region having the sequence of SEQ ID NO: 4.

In one respect, the present disclosure provides an antibody or an antigen-binding fragment thereof capable of specifically binding to human TSLP, comprising heavy chain complementary determining region 1 (HCDR1), HCDR2 and HCDR3 contained within any one of the heavy chain variable region sequence of SEQ ID NO: 1 or 3, and/or light chain complementary determining region 1 (LCDR1), LCDR2 and LCDR3 contained within any one of the heavy chain variable region sequence of SEQ ID NO: 2 or 4.

In certain embodiments, the present disclosure provides anti-TSLP antibodies and antigen-binding fragments thereof comprising HCDR1 comprising the sequence of SEQ ID NO: 11, HCDR2 comprising the sequence of SEQ ID NO: 12 or 25, and HCDR3 comprising the sequence of SEQ ID NO: 13 or 26, and/or LCDR1 comprising the sequence of SEQ ID NO: 14, LCDR2 comprising the sequence of SEQ ID NO: 15, and LCDR3 comprising the sequence of SEQ ID NO: 16.

In certain embodiments, the present disclosure provides anti-TSLP antibodies and antigen-binding fragments thereof comprising a HCDR1 comprising the sequence of SEQ ID NO: 11, a HCDR2 comprising the sequence of SEQ ID NO: 12, a HCDR3 comprising the sequence of SEQ ID NO: 13, and/or a LCDR1 comprising the sequence of SEQ ID NO: 14, a LCDR2 comprising the sequence of SEQ ID NO: 15, and a LCDR3 comprising the sequence of SEQ ID NO: 16.

In certain embodiments, the present disclosure provides anti-TSLP antibodies and antigen-binding fragments thereof comprising a HCDR1 comprising the sequence of SEQ ID NO: 11, a HCDR2 comprising the sequence of SEQ ID NO: 25, a HCDR3 comprising the sequence of SEQ ID NO: 26, and/or a LCDR1 comprising the sequence of SEQ ID NO: 14, a LCDR2 comprising the sequence of SEQ ID NO: 15, and a LCDR3 comprising the sequence of SEQ ID NO: 16.

Tables 1 and 2 below show the CDR amino acid sequences of antibodies 39H11 and 35G12, where Table 1 shows the CDR boundaries defined or identified by the convention of IMGT, and Table 2 shows the CDR boundaries defined or identified by the convention of Kabat. Table 3 below shows the heavy chain and light chain variable region amino acid sequences of antibodies 39H11 and 35G12.

TABLE 1
CDR amino acid sequences of
2 antibodies identified by IMGT

| Antibody | | CDR1 | CDR2 | CDR3 |
|---|---|---|---|---|
| 39H11 | HCDR | SEQ ID | SEQ ID NO: 12 | SEQ ID NO: 13 |
| | | NO: 11 | IFPGDGET | ARGGYVNFLY |
| | | GYAFSSYW | | AMDY |
| | LCDR | SEQ ID | SEQ ID NO: 15 | SEQ ID NO: 16 |
| | | NO: 14 | GTS | QQWSSYPLT |
| | | SSISSSN | | |
| 35G12 | HCDR | SEQ ID | SEQ ID NO: 25 | SEQ ID NO: 26 |
| | | NO: 11 | IFPGDGDT | ARSGFDYGYF |
| | | GYAFSSYW | | YAMDY |
| | LCDR | SEQ ID | SEQ ID NO: 15 | SEQ ID NO: 16 |
| | | NO: 14 | GTS | QQWSSYPLT |
| | | SSISSSN | | |

TABLE 2
CDR amino acid sequences of 2 antibodies identified by
Kabat

| Antibody | | CDR1 | CDR2 | CDR3 |
|---|---|---|---|---|
| 39H11 | HCDR | SEQ ID NO: 50 | SEQ ID NO: 51 | SEQ ID NO: 52 |
| | | SYWMN | QIFPGDGETNY | GGYVNFLYAM |
| | | | NGKFKG | DY |
| | LCDR | SEQ ID NO: 53 | SEQ ID NO: 54 | SEQ ID NO: 16 |
| | | SVSSSISSSNLH | GTSTLAS | QQWSSYPLT |
| 35G12 | HCDR | SEQ ID NO: 50 | SEQ ID NO: 56 | SEQ ID NO: 57 |
| | | SYWMN | QIFPGDGDTTY | SGFDYGYFYA |
| | | | NGNFKG | MDY |
| | LCDR | SEQ ID NO: 53 | SEQ ID NO: 58 | SEQ ID NO: 16 |
| | | SVSSSISSSNLH | GTSNLAS | QQWSSYPLT |

TABLE 3
Variable region amino acid sequences of 2 antibodies

| Antibody | VH | VL |
|---|---|---|
| 39H11 | SEQ ID NO: 1 | SEQ ID NO: 2 |
| | QVQLQQSGAELVRPGSSVKISC | EIVLTQSPALMAASPGEKVTI |
| | KASGYAFSSYWMNWVKQRPG | TCSVSSSISSSNLHWYQQKSE |
| | QGLEWIGQIFPGDGETNYNGKF | TSPKPWIYGTSTLASGVPVR |
| | KGKATLTADKSSSTAYMQLSSL | FSGSGSGTSYSLTISSMEAED |
| | TSEDSAVYFCARGGYVNFLYA | AATYYCQQWSSYPLTFGAGT |
| | MDYWGQGTSVTVSS | KLELK |
| 35G12 | SEQ ID NO: 3 | SEQ ID NO: 4 |
| | QVQLQQSGAELVRPGSSVKISC | EIVLTQSPTLMAASPGEKVTI |
| | KSSGYAFSSYWMNWVKQRPG | TCSVSSSISSSNLHWYQQKSE |
| | QGLEWIGQIFPGDGDTTYNGNF | TSPKPWIFGTSNLASGVPVRF |
| | KGKTTLTADKSSNTAYMQLSSL | SGSGSGTSYSLTISNMEAEDA |
| | TSEDSAVYFCARSGFDYGYFYA | ATYYCQQWSSYPLTFGAGTK |
| | MDYWGQGTSVTVSS | LELK |

Given that both of antibodies 39H11 and 35G12 can bind to TSLP and that antigen-binding specificity is provided primarily by the CDR1, CDR2 and CDR3 regions, the HCDR1, HCDR2 and HCDR3 sequences and LCDR1, LCDR2 and LCDR3 sequences of antibodies 39H11 and 35G12 can be “mixed and matched” (i.e., CDRs from different antibodies can be mixed and matched, but each antibody must contain a HCDR1, HCDR2 and HCDR3 and a LCDR1, LCDR2 and LCDR3) to create anti-TSLP binding molecules of the present disclosure. TSLP binding of such “mixed and matched” antibodies can be tested using the binding assays described above and in the Examples. Preferably, when VH CDR sequences are mixed and matched, the HCDR1, HCDR2 and/or HCDR3 sequence from a particular VH sequence is replaced with a structurally similar CDR sequence(s). Likewise, when VL CDR sequences are mixed and matched, the LCDR1, LCDR2 and/or LCDR3 sequence from a particular VL sequence preferably is replaced with a structurally similar CDR sequence(s). It will be readily apparent to a person skilled in the art that novel VH and VL sequences can be created by substituting one or more VH and/or VL CDR region sequences with structurally similar sequences from the CDR sequences disclosed herein for monoclonal antibodies 39H11 and 35G12.

CDRs are known to be responsible for antigen binding. However, it has been found that not all of the 6 CDRs are indispensable or unchangeable. In other words, it is possible to replace or change or modify one or more CDRs in anti-TSLP antibodies 39H11 and 35G12, yet substantially retain the specific binding affinity to TSLP.

In certain embodiments, the antibodies and antigen-binding fragments thereof provided herein comprise suitable framework region (FR) sequences, as long as the antibodies and antigen-binding fragments thereof can specifically bind to TSLP. The CDR sequences provided in Table 1 above are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

Table 4 below shows the heavy chain and light chain variable region amino acid sequences of humanized antibodies for 39H11 or 35G12, which are designated as 39H11Z07, 39H11Z11 and mAb39H11, 35G12Z01, 35G12Z02, 35G12Z03 and mAb35G12. Table 4 below shows the heavy chain and light chain variable region amino acid sequences of humanized antibodies 39H11 or 35G12, which are designated as 39H11Z07, 39H11Z11 and mAb39H11, 35G12Z01, 35G12Z02, 35G12Z03 and mAb35G12.

TABLE 4
Variable region amino acid sequences of 7 humanized
antibodies

| Antibody | VH | VL |
|---|---|---|
| 39H11Z07 | SEQ ID NO: 5 | SEQ ID NO: 6 |
| | QVQLVQSGAEVKKPGSSVKV | EIVMTQSPATLSVSPGERATL |
| | SCKASGYAFSSYWMNWVRQ | SCSVSSSISSSNLHWYQQKP |
| | APGQGLEWMGQIFPGDGETN | GQSPRPWIYGTSTLASGIPAR |
| | YNGKFKGRVTITADKSTSTAY | FSGSGSGTEFTLTISSLQSEDF |
| | MELSSLRSEDTAVYYCARGG | AVYYCQQWSSYPLTFGGGT |
| | YVNFLYAMDYWGQGTTVTV | KVEIK |
| | SS | |
| 39H11Z11 | SEQ ID NO: 7 | SEQ ID NO: 6 |
| | QVQLVQSGAEVKKPGASVKV | EIVMTQSPATLSVSPGERATL |
| | SCKASGYAFSSYWMNWVRQ | SCSVSSSISSSNLHWYQQKP |
| | APGQGLEWMGQIFPGDGETN | GQSPRPWIYGTSTLASGIPAR |
| | YNGKFKGRVTMTRDTSTSTV | FSGSGSGTEFTLTISSLQSEDF |
| | YMELSSLRSEDTAVYYCARG | AVYYCQQWSSYPLTFGGGT |
| | GYVNFLYAMDYWGQGTTVT | KVEIK |
| | VSS | |
| 35G12Z01 | SEQ ID NO: 86 | SEQ ID NO: 9 |
| | QVQLVQSGAEVKKPGASVKV | EIVMTQSPATLSVSPGERATL |
| | SCKSSGYAFSSYWMNWVRQ | SCSVSSSISSSNLHWYQQKP |
| | APGQGLEWMGQIFPGDGDTT | GQSPRPWIFGTSNLASGIPAR |
| | YNGNFKGRVTMTRDTSTSTV | FSGSGSGTEFTLTISSLQSEDF |
| | YMELSSLRSEDTAVYYCARS | AVYYCQQWSSYPLTFGGGT |
| | GFDYGYFYAMDYWGQGTTV | KVEIK |
| | TVSS | |
| 35G12Z02 | SEQ ID NO: 8 | SEQ ID NO: 9 |
| | EVQLVQSGAEVKKPGESLKIS | EIVMTQSPATLSVSPGERATL |
| | CKSSGYAFSSYWINWVRQMP | SCSVSSSISSSNLHWYQQKP |
| | GKGLEWMGQIFPGDGDTTYN | GQSPRPWIFGTSNLASGIPAR |
| | GNFKGQVTISADKSISTAYLQ | FSGSGSGTEFTLTISSLQSEDF |
| | WSSLKASDTAMYYCARSGFD | AVYYCQQWSSYPLTFGGGT |
| | YGYFYAMDYWGQGTTVTVS | KVEIK |
| | S | |
| 35G12Z03 | SEQ ID NO: 10 | SEQ ID NO: 9 |
| | QVQLVQSGAEVKKPGSSVKV | EIVMTQSPATLSVSPGERATL |
| | SCKSSGYAFSSYWINWVRQA | SCSVSSSISSSNLHWYQQKP |
| | PGQGLEWMGQIFPGDGDTTY | GQSPRPWIFGTSNLASGIPAR |
| | NGNFKGRVTITADKSTSTAY | FSGSGSGTEFTLTISSLQSEDF |
| | MELSSLRSEDTAVYYCARSGF | AVYYCQQWSSYPLTFGGGT |
| | DYGYFYAMDYWGQGTTVTV | KVEIK |
| | SS | |

TABLE 5
FR amino acid sequences of 7 humanized antibodies

| Antibody | | FR1 | FR2 | FR3 | FR4 |
|---|---|---|---|---|---|
| 39H11Z07 | HFR | SEQ ID NO: | SEQ ID NO: | SEQ ID NO: | SEQ ID NO: |
| | | 32 | 33 | 34 | 35 |
| | | QVQLVQSG | WVRQAPGQ | RVTITADKS | WGQGTTVT |
| | | AEVKKPGSS | GLEWMG | TSTAYMELS | VSS |
| | | VKVSCKAS | | SLRSEDTAV | |
| | | | | YYC | |
| | LFR | SEQ ID NO: | SEQ ID NO: | SEQ ID NO: | SEQ ID NO: |
| | | 36 | 37 | 38 | 39 |
| | | EIVMTQSPA | WYQQKPGQ | GIPARFSGS | FGGGTKVEI |
| | | TLSVSPGER | SPRPWIY | GSGTEFTLT | K |
| | | ATLSC | | ISSLQSEDF | |
| | | | | AVYYC | |
| 39H11Z11 | HFR | SEQ ID NO: | SEQ ID NO: | SEQ ID NO: | SEQ ID NO: |
| | | 40 | 33 | 41 | 35 |
| | | QVQLVQSG | WVRQAPGQ | RVTMTRDT | WGQGTTVT |
| | | AEVKKPGA | GLEWMG | STSTVYMEL | VSS |
| | | SVKVSCKA | | SSLRSEDTA | |
| | | S | | VYYC | |
| | LFR | SEQ ID NO: | SEQ ID NO: | SEQ ID NO: | SEQ ID NO: |
| | | 36 | 37 | 38 | 39 |
| | | EIVMTQSPA | WYQQKPGQ | GIPARFSGS | FGGGTKVEI |
| | | TLSVSPGER | SPRPWIY | GSGTEFTLT | K |
| | | ATLSC | | ISSLQSEDF | |
| | | | | AVYYC | |
| 35G12Z01 | HFR | SEQ ID NO: | SEQ ID NO: | SEQ ID NO: | SEQ ID NO: |
| | | 87 | 33 | 41 | 35 |
| | | QVQLVQSG | WVRQAPGQ | RVTMTRDT | WGQGTTVT |
| | | AEVKKPGA | GLEWMG | STSTVYMEL | VSS |
| | | SVKVSCKSS | | SSLRSEDTA | |
| | | | | VYYC | |
| | LFR | SEQ ID NO: | SEQ ID NO: | SEQ ID NO: | SEQ ID NO: |
| | | 36 | 45 | 38 | 39 |
| | | EIVMTQSPA | WYQQKPGQ | GIPARFSGS | FGGGTKVEI |
| | | TLSVSPGER | SPRPWIF | GSGTEFTLT | K |
| | | ATLSC | | ISSLQSEDF | |
| | | | | AVYYC | |
| 35G12Z02 | HFR | SEQ ID NO: | SEQ ID NO: | SEQ ID NO: | SEQ ID NO: |
| | | 42 | 43 | 44 | 35 |
| | | EVQLVQSG | WVRQMPG | QVTISADKS | WGQGTTVT |
| | | AEVKKPGE | KGLEWMG | ISTAYLQWS | VSS |
| | | SLKISCKSS | | SLKASDTA | |
| | | | | MYYC | |
| | LFR | SEQ ID NO: | SEQ ID NO: | SEQ ID NO: | SEQ ID NO: |
| | | 36 | 45 | 38 | 39 |
| | | EIVMTQSPA | WYQQKPGQ | GIPARFSGS | FGGGTKVEI |
| | | TLSVSPGER | SPRPWIF | GSGTEFTLT | K |
| | | ATLSC | | ISSLQSEDF | |
| | | | | AVYYC | |
| 35G12Z03 | HFR | SEQ ID NO: | SEQ ID NO: | SEQ ID NO: | SEQ ID NO: |
| | | 47 | 48 | 49 | 35 |
| | | QVQLVQSG | WVRQAPGQ | RVTITADKS | WGQGTTVT |
| | | AEVKKPGSS | GLEWMG | TSTAYMELS | VSS |
| | | VKVSCKSS | | SLRSEDTAV | |
| | | | | YYC | |
| | LFR | SEQ ID NO: | SEQ ID NO: | SEQ ID NO: | SEQ ID NO: |
| | | 36 | 45 | 38 | 39 |
| | | EIVMTQSPA | WYQQKPGQ | GIPARFSGS | FGGGTKVEI |
| | | TLSVSPGER | SPRPWIF | GSGTEFTLT | K |
| | | ATLSC | | ISSLQSEDF | |
| | | | | AVYYC | |

In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1-4, and/or light chain LFR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In certain embodiments, the present disclosure also provides humanized anti-TSLP antibodies and antigen-binding fragments thereof comprising a heavy chain HFR1 comprising the sequence of X₂₁VQLVQSGAEVKKPGX₂₂SX₂₃KX₂₄SCKX₂₅S (SEQ ID NO: 65) or a homologous sequence of at least 80% sequence identity thereof, a heavy chain HFR2 comprising the sequence of WVRQX₂₇PGX₂₈GLEWMG (SEQ ID NO: 66) or a homologous sequence of at least 80% sequence identity thereof, a heavy chain HFR3 comprising the sequence of X₃₁VTX₃₂X₃₃X₃₄DX₃₅SX₃₆STX₃₇YX₃₈X₃₉X₄₀SSLX₄₁X₄₂X₄₃DTAX₄₄YYC (SEQ ID NO: 67) or a homologous sequence of at least 80% sequence identity thereof, and a heavy chain HFR4 comprising the sequence of SEQ ID NO: 35 or a homologous sequence of at least 80% sequence identity thereof, wherein X₂₁ is E or Q; X₂₂ is E, S or A; X₂₃ is L or V; X₂₄ is I or V; X₂₅ is S or A; X₂₇ is M or A; X₂₈ is K or Q; X₃₁ is Q or R; X₃₂ is I or M; X₃₃ is S or T; X₃₄ is A or R; X₃₅ is K or T; X₃₆ is I or T; X₃₇ is A or V; X₃₈ is L or M; X₃₉ is Q or E; X₄₀ is W or L; X₄₁ is K or R; X₄₂ is A or S; X₄₃ is S or E; X₄₄ is M or V.

In certain embodiments, the present disclosure also provides humanized anti-TSLP antibodies and antigen-binding fragments thereof comprising a light chain LFR1 comprising the sequence of SEQ ID NO: 36 or a homologous sequence of at least 80% sequence identity thereof, a light chain LFR2 comprising the sequence of WYQQKPGQSPRPWIX₄₅ (SEQ ID NO: 68) or a homologous sequence of at least 80% sequence identity thereof, a light chain LFR3 comprising the sequence of SEQ ID NO: 38 or a homologous sequence of at least 80% sequence identity thereof, and a light chain LFR4 comprising the sequence of SEQ ID NO: 39 or a homologous sequence of at least 80% sequence identity thereof, wherein X₄₅ is Y or F.

In certain embodiments, the present disclosure also provides humanized anti-TSLP antibodies and antigen-binding fragments thereof comprising a heavy chain HFR1 comprising a sequence selected from the group consisting of SEQ ID NOs: 32, 40, 42, 47 and 87, a heavy chain HFR2 comprising a sequence selected from the group consisting of SEQ ID NOs: 33, 43 and 48, a heavy chain HFR3 comprising a sequence selected from the group consisting of SEQ ID NOs: 34, 41, 44 and 49, and a heavy chain HFR4 comprising a sequence of SEQ ID NO: 35; and/or a light chain LFR1 comprising a sequence from the group consisting of SEQ ID NO: 36, a light chain LFR2 comprising a sequence selected from the group consisting of SEQ ID NOs: 37 and 45, a light chain LFR3 comprising a sequence of SEQ ID NO: 38, and a light chain LFR4 comprising a sequence of SEQ ID NO: 39.

In certain embodiments, the present disclosure also provides humanized anti-TSLP antibodies and antigen-binding fragments thereof comprising HFR1, HFR2, HFR3, and/or HFR4 sequences contained in a heavy chain variable region selected from a group consisting of: 39H11Z07-VH (SEQ ID NO: 5), 39H11Z11-VH (SEQ ID NO: 7), 35G12Z01-VH (SEQ ID NO: 86), 35G12Z02-VH (SEQ ID NO: 8), and 35G12Z03-VH (SEQ ID NO: 10).

In certain embodiments, the present disclosure also provides humanized anti-TSLP antibodies and antigen-binding fragments thereof comprising LFR1, LFR2, LFR3, and/or LFR4 sequences contained in a light chain variable region selected from a group consisting of: 39H11Z07-VL/39H11Z11-VL (SEQ ID NO: 6), 35G12Z01-VL/35G12Z03-VL (SEQ ID NO: 9).

In certain embodiments, the humanized anti-TSLP antibodies and antigen-binding fragments thereof provided herein comprise a heavy chain variable domain sequence selected from the group consisting of SEQ ID NO: 5, SEQ ID NO: 7, SEQ ID NO: 8, SEQ ID NO: 86, and SEQ ID NO: 10; and/or a light chain variable domain sequence selected from the group consisting of SEQ ID NO: 6 and SEQ ID NO: 9.

The present disclosure also provides exemplary humanized antibodies of 39H11 or 35G12, including:
1) “39H11Z07” comprising the heavy chain variable region of 39H11Z07-VH (SEQ ID NO: 5) and the light chain variable region of 39H11Z07-VL (SEQ ID NO: 6);
2) “mAb39H11” comprising the heavy chain variable region of 39H11Z07-VH (SEQ ID NO: 5) and the light chain variable region of 39H11Z07-VL (SEQ ID NO: 6), and further comprising a Fc region of IgG4 with PAA-YTE mutations (SEQ ID NO: 80);
3) “39H11Z11” comprising the heavy chain variable region of 39H11Z11-VH (SEQ ID NO: 7), and the light chain variable region of 39H11Z11-VL (SEQ ID NO: 6);
4) “35G12Z01” comprising the heavy chain variable region of 35G12Z01-VH (SEQ ID NO: 86), and the light chain variable region of 35G12Z01-VL (SEQ ID NO: 9);
5) “35G12Z02” comprising the heavy chain variable region of 35G12Z02-VH (SEQ ID NO: 8), and the light chain variable region of 35G12Z02-VL (SEQ ID NO: 9);
6) “35G12Z03” comprising the heavy chain variable region of 35G12Z03-VH (SEQ ID NO: 10), and the light chain variable region of 35G12Z03-VL (SEQ ID NO: 9);
7) “mAb35G12” comprising the heavy chain variable region of 35G12Z03-VH (SEQ ID NO: 10), and the light chain variable region of 35G12Z03-VL (SEQ ID NO: 9), and further comprising an Fc region of IgG4 with PAA-YTE mutations (SEQ ID NO: 75).
8) “mAb35G12_IgG1” comprising the heavy chain variable region of 35G12Z03-VH (SEQ ID NO: 10), and the light chain variable region of 35G12Z03-VL (SEQ ID NO: 9), and further comprising an Fc region of IgG1 (SEQ ID NO: 88).

These exemplary humanized anti-TSLP antibodies retained the specific binding capacity or affinity to TSLP, and are at least comparable to, or even better than, the parent mouse antibody 39H11 or 35G12 in that aspect. For example, data is provided in Example 7.

In some embodiments, the anti-TSLP antibodies and antigen-binding fragments provided herein comprise all or a portion of the heavy chain variable domain and/or all or a portion of the light chain variable domain. In one embodiment, the anti-TSLP antibody or an antigen-binding fragment thereof provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-TSLP antibodies or the antigen-binding fragments thereof provided herein further comprise an immunoglobulin (Ig) constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the anti-TSLP antibodies or the antigen-binding fragments thereof provided herein comprises heavy chain constant regions of human IgG1, IgG2, IgG3, or IgG4. In certain embodiments, the light chain constant region comprises Cκ or Cλ. The constant region of the anti-TSLP antibodies or the antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the anti-TSLP antibodies and antigen-binding fragments thereof provided herein comprises an Fc region of IgG1 or IgG3 isotype, which could induce ADCC or CDC; or alternatively, a constant region of IgG4 or IgG2 isotype, which has reduced or depleted effector function. In certain embodiments, the anti-TSLP antibodies or antigen-binding fragments thereof provided herein comprise a wild type human IgG4 Fc region or other wild type human IgG4 alleles. In certain embodiments, the anti-TSLP antibodies or antigen-binding fragments thereof provided herein comprise a human IgG4 Fc region comprising one or more mutations selected from the group consisting of S228P, F234A, L235A, M252Y, S254T, T256E and K447del. In certain embodiments, the anti-TSLP antibodies or antigen-binding fragments thereof provided herein comprise a human IgG4 Fc region comprising mutations of S228P, F234A, L235A, M252Y, S254T, T256E and K447del. In certain embodiments, the anti-TSLP antibodies or antigen-binding fragments thereof provided herein comprise the heavy chain constant region with an amino acid sequence as shown in SEQ ID NO: 75. In certain embodiments, the anti-TSLP antibodies or antigen-binding fragments thereof provided herein comprise a human IgG1 Fc region comprising one or more mutations selected from the group consisting of L234A, L235A, M252Y, S254T and T256E. In certain embodiments, the anti-TSLP antibodies or antigen-binding fragments thereof provided herein comprise a human IgG1 Fc region comprising mutations of L234A, L235A, M252Y, S254T and T256E. In certain embodiments, the anti-TSLP antibodies or antigen-binding fragments thereof provided herein comprise the heavy chain constant region with an amino acid sequence as shown in SEQ ID NO: 88.

In certain embodiments, the antibodies or the antigen-binding fragments thereof provided herein further comprising a signal peptide at the N-terminal of the heavy chain variable region and/or a signal peptide at the N-terminal of the light chain variable region. In certain embodiments, the signal peptide comprises an amino acid sequence of SEQ ID NO: 76 (MGWSCIILFLVATATGVHS).

In certain embodiments, the antibodies or the antigen-binding fragments thereof provided herein have a specific binding affinity to human TSLP which is sufficient to provide for diagnostic and/or therapeutic use.

The antibodies or antigen-binding fragments thereof provided herein can be a monoclonal antibody, a polyclonal antibody, a humanized antibody, a chimeric antibody, a recombinant antibody, a bispecific antibody, a multi-specific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides an anti-TSLP antibody or antigen-binding fragment thereof, which does not compete for binding to human TSLP with an antibody comprising a heavy chain variable region comprising the sequence of SEQ ID NO: 77, and a light chain variable region comprising the sequence of SEQ ID NO: 78, wherein the antibody or an antigen-binding fragment thereof of is not AMG157.

“AMG157” as used herein refers to an antibody or antigen binding fragment thereof comprising a heavy chain variable region having an amino acid sequence of SEQ ID NO: 77, and a light chain variable region having an amino acid sequence of SEQ ID NO: 78.

### Antibody Variants

The antibodies and antigen-binding fragments thereof provided herein also encompass various variants of the antibody sequences provided herein.

In certain embodiments, the antibody variants comprise one or more modifications or substitutions in one or more of the CDR sequences as provided in Tables 1 and 2 above, one or more of the non-CDR sequences of the heavy chain variable region or light chain variable region provided in Tables 3 and 4 above, and/or the constant region (e.g., Fc region). Such variants retain binding specificity to TSLP of their parent antibodies but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g., one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

### Affinity Variants

Affinity variants of antibodies may contain modifications or substitutions in one or more CDR sequences as provided in Tables 1 and 2 above, one or more FR sequences as provided in Table 5 above, or the heavy or light chain variable region sequences provided in Tables 3 and 4 above. FR sequences can be readily identified by a person skilled in the art based on the CDR sequences in Tables 1 and 2 above and variable region sequences in Tables 3 and 4 above, as it is well-known in the art that a CDR region is flanked by two FR regions in the variable region. The affinity variants retain specific binding affinity to TSLP of the parent antibody, or even have improved TSLP specific binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Tables 1 and 2 above, and variable region sequences provided in Tables 3 and 4 above, one or more amino acid residues may be substituted yet the resulting antibody or antigen-binding fragment still retain the binding affinity or binding capacity to TSLP, or even have an improved binding affinity or capacity. Various methods known in the art can be used to achieve this purpose. For example, a library of antibody variants (such as Fab or scFv variants) can be generated and expressed with phage display technology, and then screened for the binding affinity to human TSLP. For another example, computer software can be used to virtually simulate the binding of the antibodies to human TSLP and identify the amino acid residues on the antibodies which form the binding interface. Such residues may be either avoided in the substitution so as to prevent reduction in binding affinity or targeted for substitution to provide for a stronger binding.

In certain embodiments, the humanized antibody or antigen-binding fragment thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitution(s) in the CDR sequences and/or FR sequences in total.

In certain embodiments, the anti-TSLP antibodies or antigen-binding fragments thereof comprise 1, 2, or 3 CDR sequences having at least 80% (e.g., at least 85%, 88%, 90%, 91%, 92%, 93%, 94%, 95%, 96%, 97%, 98%, 99%) sequence identity to that (or those) listed in Tables 1 and 2 above yet retaining the specific binding affinity to TSLP at a level similar to or even higher than its parent antibody.

In certain embodiments, the anti-TSLP antibodies or antigen-binding fragments thereof comprise one or more variable region sequences having at least 80% (e.g., at least 85%, 88%, 90%, 91%, 92%, 93%, 94%, 95%, 96%, 97%, 98%, 99%) sequence identity to that (or those) listed in Tables 3 and 4 above yet retaining the specific binding affinity to TSLP at a level similar to or even higher than its parent antibody. In some embodiments, a total of 1 to 10 amino acids have been substituted, inserted, or deleted in a variable region sequence listed in Tables 3 and 4 above. In some embodiments, the substitutions, insertions, or deletions occur in regions outside the CDRs (e.g., in the FRs).

### Glycosylation Variants

The anti-TSLP antibodies or antigen-binding fragments thereof provided herein also encompass glycosylation variants, which can be obtained to either increase or decrease the extent of glycosylation of the antibodies or antigen binding fragments thereof.

The antibodies or antigen binding fragments thereof may comprise one or more modifications that introduce or remove a glycosylation site. A glycosylation site is an amino acid residue with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

In certain embodiments, the anti-TSLP antibodies and antigen-binding fragments provided herein comprise a mutation at N297 (e.g., N297A, N297Q, or N297G) to remove the glycosylation site.

### Cysteine-Engineered Variants

The anti-TSLP antibodies or antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### Fc Variants

The anti-TSLP antibodies or antigen-binding fragments thereof provided herein also encompass Fc variants, which comprise one or more amino acid residue modifications or substitutions at the Fc region and/or hinge region, for example, to provide for altered effector functions such as ADCC and CDC. Methods of altering ADCC activity by antibody engineering have been described in the art, see for example, Shields R L. et al., J Biol Chem. 2001. 276 (9): 6591-604; Idusogie E E. et al., J Immunol. 2000.164 (8): 4178-84; Steurer W. et al., J Immunol. 1995, 155 (3): 1165-74; Idusogie E E. et al., J Immunol. 2001, 166 (4): 2571-5; Lazar G A. et al., PNAS, 2006, 103 (11): 4005-4010; Ryan M C. et al., Mol. Cancer Ther., 2007, 6:3009-3018; Richards J O, et al., Mol Cancer Ther. 2008, 7 (8): 2517-27; Shields R. L. et al., J. Biol. Chem, 2002, 277:26733-26740; Shinkawa T. et al., J. Biol. Chem, 2003, 278:3466-3473.

CDC activity of the antibodies or antigen-binding fragments provided herein can also be altered, for example, by improving or diminishing C1q binding and/or CDC (see, for example, WO99/51642; Duncan & Winter Nature 322:738-40 (1988); U.S. Pat. Nos. 5,648,260; 5,624,821; and WO94/29351 concerning other examples of Fc region variants). One or more amino acids selected from amino acid residues 329, 331 and 322 of the Fc region can be replaced with a different amino acid residue to alter C1q binding and/or reduced or abolished complement dependent cytotoxicity (CDC) (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-TSLP antibodies or antigen-binding fragments thereof provided herein has reduced effector functions, and comprise one or more amino acid substitution(s) in IgG1 at a position selected from the group consisting of: 234, 235, 237, and 238, 268, 297, 309, 330, and 331. In certain embodiments, the anti-TSLP antibodies or antigen-binding fragments thereof provided herein is of IgG1 isotype and comprise one or more amino acid substitution(s) selected from the group consisting of: N297A, N297Q, N297G, L235E, L234A, L235A, L234F, L235E, P331S, and any combination thereof. In certain embodiments, the anti-TSLP antibodies or antigen-binding fragments thereof provided herein is of IgG2 isotype, and comprises one or more amino acid substitution(s) selected from the group consisting of: H268Q, V309L, A330S, P331S, V234A, G237A, P238S, H268A, and any combination thereof (e.g., H268Q/V309L/A330S/P331S, V234A/G237A/P238S/H268A/V309L/A330S/P331S). In certain embodiments, the anti-TSLP antibodies or antigen-binding fragments thereof provided herein is of IgG4 isotype, and comprises one or more amino acid substitution(s) selected from the group consisting of: N297A, N297Q, N297G, L235E, L234A, L235A, M252Y/S254T/T256E, T307Q/N434 and any combination thereof. In certain embodiments, the anti-TSLP antibodies or antigen-binding fragments thereof provided herein is of IgG2/IgG4 cross isotype. Examples of IgG2/IgG4 cross isotype is described in Rother R P et al., Nat Biotechnol 25:1256-1264 (2007).

In certain embodiments, the anti-TSLP antibodies and antigen-binding fragments provided herein is of IgG4 isotype and comprises the amino acid substitutions of S228P/F234A/L235A (PAA). In certain embodiments, the anti-TSLP antibodies and antigen-binding fragments provided herein is of IgG4 isotype and comprises the amino acid substitutions of M252Y/S254T/T256 (YTE). In certain embodiments, the anti-TSLP antibodies and antigen-binding fragments provided herein is of IgG4 isotype and comprises the amino acid deletion of K447del.

In certain embodiments, the anti-TSLP antibodies and antigen-binding fragments provided herein is of IgG4 isotype and comprises one or more amino acid substitution(s), for example at the point of 228. In certain embodiments, the anti-TSLP antibodies and antigen-binding fragments provided herein is of IgG4 isotype and comprises one or more mutations selected from the group consisting of S228P, F234A, L235A, M252Y, S254T, T256E and K447del in the Fc region, or any combination thereof. In certain embodiments, the anti-TSLP antibodies or antigen-binding fragments thereof provided herein comprise the heavy chain constant region with an amino acid sequence as shown in SEQ ID NO: 75.

In certain embodiments, the anti-TSLP antibodies and antigen-binding fragments provided herein is of IgG1 isotype and comprises the amino acid substitutions of L234A/L235A (LALA). In certain embodiments, the anti-TSLP antibodies and antigen-binding fragments provided herein is of IgG1 isotype and comprises the amino acid substitutions of M252Y/S254T/T256 (YTE). In certain embodiments, the anti-TSLP antibodies and antigen-binding fragments provided herein is of IgG1 isotype and comprises one or more mutations selected from the group consisting of L234A, L235A, M252Y, S254T and T256E in the Fc region, or any combination thereof. In certain embodiments, the anti-TSLP antibodies or antigen-binding fragments thereof provided herein comprise the heavy chain constant region with an amino acid sequence as shown in SEQ ID NO: 88.

In certain embodiments, the anti-TSLP antibodies or antigen-binding fragments thereof comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6 (1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70:3269-3277 (2010); and Hinton, P. et al., J. Immunology, 176:346-356 (2006).

In certain embodiments, anti-TSLP antibodies or antigen-binding fragments thereof comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

### Antigen-Binding Fragments

Provided herein are also anti-TSLP antigen-binding fragments. Various types of antigen-binding fragments are known in the art and can be developed based on the anti-TSLP antibodies provided herein, including for example, the exemplary antibodies whose CDRs are shown in Tables 1 and 2 above, and variable sequences are shown in Tables 3 and 4 above, and their different variants (such as affinity variants, glycosylation variants, Fc variants, cysteine-engineered variants and so on).

In certain embodiments, an anti-TSLP antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

Various techniques can be used for the production of such antigen-binding fragments. Illustrative methods include, enzymatic digestion of intact antibodies (see, e.g. Morimoto et al., Journal of Biochemical and Biophysical Methods 24:107-117 (1992); and Brennan et al., Science, 229:81 (1985)), recombinant expression by host cells such as E. Coli (e.g. for Fab, Fv and ScFv antibody fragments), screening from a phage display library as discussed above (e.g. for ScFv), and chemical coupling of two Fab′-SH fragments to form F(ab′)₂ fragments (Carter et al., Bio Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the anti-TSLP antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e., having the same sequences), or structurally different (i.e., having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific Antibodies

In certain embodiments, the anti-TSLP antibodies or antigen-binding fragments thereof is bispecific. In certain embodiments, the antibody or antigen-binding fragment thereof is further linked to a second functional moiety having a different binding specificity from said TSLP antibody, or antigen binding fragment thereof.

In certain embodiments, the bispecific antibodies or antigen-binding fragments thereof provided herein are capable of specifically binding to a second antigen other than TSLP, or a second epitope on TSLP. In some embodiments, the second antigen other than TSLP is selected from the group consisting of IL-33, IL-25, IL-4, IL-5, IL-4R, IL-13 or the like.

### Conjugates

In some embodiments, the anti-TSLP antibodies or antigen-binding fragments thereof further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding, affinity binding, intercalation, coordinate binding, complexation, association, blending, or addition, among other methods. In some embodiments, the antibodies or antigen-binding fragments thereof can be linked to one or more conjugates via a linker.

In certain embodiments, the antibodies or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In some embodiments, the antibody moiety is linked with the conjugate moiety through a chemical bond or a linker. In some embodiments, the antibody moiety and the conjugate moiety is linked using a variety of well-known bifunctional reagents and chemistries suitable for conjugating to proteins. Such reagents include but are not limited to: N-succinimidyl-3-(2-pyridyldithio) propionate (SPDP), succinimidyl-4-(N-maleimidornethyl) cyclohexane-1-carboxylate (SMCC), iminothiolane (IT), bifunctional derivatives of imidoesters (e.g., dimethyl adipimidate HQ), active esters (e.g., disuccinimidyl suberate), aldehydes (e.g., glutaraldehyde), bis-azido compounds bis-(p-azidobenzoyl)-hexane-diamine), bis-diazonium derivatives (e.g., bis-(p-diazoniumbenzoyl)-ethylenediamine), diisocyanates (e.g., toluene-2,6-diisocyanate), and bis-active fluorine compounds (e.g., 1,5-difluoro-2,4-dinitrobenzene).

In certain embodiments, the antibodies or antigen-binding fragments thereof may be linked to a conjugate moiety indirectly, or through another conjugate moiety. For example, the antibodies or antigen-binding fragments thereof provided herein may be conjugated to biotin, then indirectly conjugated to a second conjugate that is conjugated to avidin. In some embodiments, the conjugate moiety comprises a clearance-modifying agent (e.g., a polymer such as PEG which extends half-life), a chemotherapeutic agent, a toxin, a radioactive isotope, a lanthanide, a detectable label (e.g., a luminescent label, a fluorescent label, an enzyme-substrate label), a DNA-alkylator, a topoisomerase inhibitor, a tubulin-binder, a purification moiety or other therapeutic agents or drugs.

The therapeutic agents or drugs useful as the conjugate moiety can be those which are useful for treating asthma, Polyp nasal Sinusitis, chronic obstructive pulmonary disease (COPD), urticaria, eosinophilic esophagitis (EoE), and atopic dermatitis and the like.

In some embodiments, the conjugate moiety comprises a therapeutic agent or drug for treating asthma. In some embodiments, the conjugate moiety comprises beta2-adrenoceptor agonists (SABA, such as salbutamol, salmeterol, formoterol and the like), adrenergic agonists (such as inhaled epinephrine), corticosteroids (preferably inhaled forms such as beclomethasone), leukotriene receptor antagonists (such as montelukast and zafirlukast), mast cell stabilizers (such as cromolyn sodium), macrolide antibiotics, or a combination thereof.

In some embodiments, the conjugate moiety comprises a therapeutic agent or drug for treating Polyp nasal Sinusitis. In some embodiments, the conjugate moiety comprises antibiotics, and small molecules like kinase inhibitor, steroid, etc.

In some embodiments, the conjugate moiety comprises a therapeutic agent or drug for treating COPD. In some embodiments, the conjugate moiety comprises antibiotics (including amoxicillin, doxycycline and azithromycin), beta2-adrenergic agonists, anticholinergics (such as ipratropium and tiotropium), long-acting muscarinic antagonist (LAMA, such as tiotropium), aclidinium, LAMA umeclidinium bromide, corticosteroids, phosphodiesterase-4 inhibitors (PDE4 inhibitors, such as roflumilast) and the like.

In some embodiments, the conjugate moiety comprises a therapeutic agent or drug for treating urticaria. In some embodiments, the conjugate moiety comprises antihistamines, systemic steroids, leukotriene-receptor antagonists, anti-inflammatory medications, omalizumab, immunosuppressants, or the like. Non-limiting exemplary antihistamines include diphenhydramine, hydroxyzine, loratadine, cetirizine, desloratadine, fexofenadine and the like.

In some embodiments, the conjugate moiety comprises a therapeutic agent or drug for treating EoE. In some embodiments, the conjugate moiety comprises proton-pump inhibitors (PPIs), corticosteroids, and the like.

In some embodiments, the conjugate moiety comprises a therapeutic agent or drug for treating atopic dermatitis. In some embodiments, the conjugate moiety comprises corticosteroids (such as hydrocortisone), calcineurin inhibitors (such as tacrolimus or pimecrolimus), tacrolimus, pimecrolimus, PDE-4 inhibitor (such as crisaborole), systemic immunosuppressants (such as ciclosporin, methotrexate, interferon gamma-1b, mycophenolate mofetil, and azathioprine), additional antibodies (such as dupilumab, tralokinumab), and the like.

In some embodiments, the conjugate moiety comprises an enzymatically active toxin or a fragment thereof, including but not limited to diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins, Momordica charantia inhibitor, curcin, crotin, Sapaonaria officinalis inhibitor, gelonin, mitogellin, restrictocin, phenomycin, enomycin, and the tricothecenes.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, and anthramycin (AMC)), anti-mitotic agents (e.g. vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioactive isotopes, luminescent labels, chromophoric moieties, digoxigenin, biotin/avidin, DNA molecules or gold for detection. A variety of radioactive isotopes are available for the production of such radio-conjugates. Examples include ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb, and radioactive isotopes of Lu. In some embodiments, the conjugate moiety may comprise a radioisotope for scintigraphic detection, or a spin label for NMR detection or MRI. Suitable radioisotopes or spin labels can include, as ¹²³I, ¹³¹I, ¹¹¹In, ¹³C, ¹⁹F, ¹⁵N, ¹⁷O, various isotopes of Gd, Mn, and Fe.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight and may be branched or unbranched. The number of polymers attached to the antibody may vary, and if more than one polymer is attached, they can be the same or different molecules.

In certain embodiments, the conjugate moiety can be a purification moiety such as a magnetic bead.

In certain embodiments, the antibodies or antigen-binding fragments thereof provided herein is used as a base for a conjugate.

### Polynucleotides and Recombinant Methods

The present disclosure provides isolated polynucleotides that encode the anti-TSLP antibodies or antigen-binding fragments thereof provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-TSLP antibodies or antigen-binding fragments thereof can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1α), and a transcription termination sequence.

The present disclosure provides vectors comprising the isolated polynucleotide provided herein. In certain embodiments, the polynucleotide provided herein encodes the antibodies or antigen-binding fragments thereof, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT.RTM., pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-TSLP antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for anti-TSLP antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or an antigen-binding fragment thereof provided herein, comprising culturing the host cell provided herein under the condition at which the vector of the present disclosure is expressed. The host cells used to produce the antibodies or antigen-binding fragments thereof provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-TSLP antibodies or antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Composition

The present disclosure further provides pharmaceutical compositions comprising the anti-TSLP antibodies or antigen-binding fragments thereof and one or more pharmaceutically acceptable carriers.

The present disclosure further provides a pharmaceutical composition comprising the polynucleotides encoding the anti-TSLP antibodies or antigen-binding fragments thereof, and one or more pharmaceutically acceptable carriers. Antibodies provided herein can also be produced in vivo by delivery of polynucleotides encoding the antibodies or antigen-binding fragments thereof provided herein, such as, for example, in-vitro-transcribed mRNA, or expression vectors. Methods are known in the art for polynucleotide delivery for antibody expression in vivo, see, for example, Rybakova, Y. et al, Molecular Therapy, [symbol] vol. 27 (8), pp. 1415-1423 (2019); Deal, C. E. et al, Vaccines, 2021, 9, 108.

The present disclosure further provides pharmaceutical compositions comprising an expression vector comprising the polynucleotides encoding the anti-TSLP antibodies or antigen-binding fragments thereof, and one or more pharmaceutically acceptable carriers.

In certain embodiments, the expression vector comprises a viral vector or a non-viral vector. Examples of viral vectors include, without limitation, adeno-associated virus (AAV) vector, lentivirus vector, retrovirus vector, and adenovirus vector. Examples of non-viral vectors include, without limitation, naked DNA, plasmid, exosome, mRNA, and so on. In certain embodiments, the expression vector is suitable for gene therapy in human. Suitable vectors for gene therapy include, for example, adeno-associated virus (AAV), or adenovirus vector. In certain embodiments, the expression vector comprises a DNA vector or an RNA vector. In certain embodiments, the pharmaceutically acceptable carriers are polymeric excipients, such as without limitation, microspheres, microcapsules, polymeric micelles and dendrimers. The polynucleotides, or polynucleotide vectors of the present disclosure may be encapsulated, adhered to, or coated on the polymer-based components by methods known in the art (see for example, W. Heiser, Nonviral gene transfer techniques, published by Humana Press, 2004; U.S. Pat. No. 6,025,337; Advanced Drug Delivery Reviews, 57 (15): 2177-2202 (2005)).

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the anti-TSLP antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given and can be empirically determined.

### Kits

In certain embodiments, the present disclosure provides a kit comprising the antibody or an antigen-binding fragment thereof provided herein. In certain embodiments, the present disclosure provides a kit comprising the antibody or an antigen-binding fragment thereof provided herein, and a second therapeutic agent.

The therapeutic agents or drugs useful as the conjugate moiety can be those which are useful for treating asthma, Polyp nasal Sinusitis, chronic obstructive pulmonary disease (COPD), urticaria, eosinophilic esophagitis (EoE), and atopic dermatitis, and the like.

In some embodiments, the second therapeutic agent comprises a therapeutic agent or drug for treating asthma. In some embodiments, the conjugate moiety comprises beta2-adrenoceptor agonists (SABA, such as salbutamol, salmeterol, formoterol and the like), adrenergic agonists (such as inhaled epinephrine), corticosteroids (preferably inhaled forms such as beclomethasone), leukotriene receptor antagonists (such as montelukast and zafirlukast), mast cell stabilizers (such as cromolyn sodium), macrolide antibiotics, or a combination thereof.

In some embodiments, the second therapeutic agent comprises a therapeutic agent or drug for treating Polyp nasal Sinusitis. In some embodiments, the conjugate moiety comprises antibiotics, and small molecules like kinase inhibitor, steroid, etc.

In some embodiments, the second therapeutic agent comprises a therapeutic agent or drug for treating COPD. In some embodiments, the conjugate moiety comprises antibiotics (including amoxicillin, doxycycline and azithromycin), beta2-adrenergic agonists, anticholinergics (such as ipratropium and tiotropium), long-acting muscarinic antagonist (LAMA, such as tiotropium), aclidinium, LAMA umeclidinium bromide, corticosteroids, phosphodiesterase-4 inhibitors (PDE4 inhibitors, such as roflumilast) and the like. In some embodiments, the second therapeutic agent is a monoclonal antibody such as Dupilumab or Itepekimab.

In some embodiments, the second therapeutic agent comprises a therapeutic agent or drug for treating urticaria. In some embodiments, the conjugate moiety comprises antihistamines, systemic steroids, leukotriene-receptor antagonists, anti-inflammatory medications, omalizumab, immunosuppressants, or the like. Non-limiting exemplary antihistamines include diphenhydramine, hydroxyzine, loratadine, cetirizine, desloratadine, fexofenadine and the like. In some embodiments, the second therapeutic agent is a monoclonal antibody such as Dupilumab or Itepekimab.

In some embodiments, the second therapeutic agent comprises a therapeutic agent or drug for treating EoE. In some embodiments, the conjugate moiety comprises proton-pump inhibitors (PPIs), corticosteroids, and the like. In some embodiments, the second therapeutic agent is a monoclonal antibody such as Dupilumab or Itepekimab.

In some embodiments, the second therapeutic agent comprises a therapeutic agent or drug for treating atopic dermatitis. In some embodiments, the conjugate moiety comprises corticosteroids (such as hydrocortisone), calcineurin inhibitors (such as tacrolimus or pimecrolimus), tacrolimus, pimecrolimus, PDE-4 inhibitor (such as crisaborole), systemic immunosuppressants (such as ciclosporin, methotrexate, interferon gamma-1b, mycophenolate mofetil, and azathioprine), additional antibodies (such as dupilumab, tralokinumab), and the like. In some embodiments, the second therapeutic agent is a monoclonal antibody such as Dupilumab or Itepekimab.

In some embodiments, the second therapeutic agent is a drug targeting IL-33, IL-25, IL-4, IL-5, IL-4R, IL-13, etc.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

### Chimeric Antigen Receptor (CAR) Composition

The present disclosure also provides chimeric antigen receptors (CARs) comprising an anti-TSLP antigen binding domain as provided herein and a T-cell activation domain. Chimeric antigen receptors (CARs) are engineered chimeric receptors that combine an antigen-binding domain of an antibody with one or more signaling domains for T cell activation. Immune cells such as T cells and Nature Killer (NK) cells can be genetically engineered to express CARs. T cells expressing a CAR are referred to as CAR-T cells. CAR can mediate antigen-specific cellular immune activity in the T cells, enabling the CAR-T cells to eliminate cells (e.g., tumor cells) expressing the targeted antigen. In one embodiment, binding of the CAR-T cells provided herein to TSLP expressed on cells such as cancer cells, results in proliferation and/or activation of said CAR-T cells, wherein said activated CAT-T cells can release cytotoxic factors, e.g., perforin, granzymes, and granulysin, and initiate cytolysis and/or apoptosis of the cancer cells.

In some embodiments, the T-cell activation domain of the CAR comprises a co-stimulatory signaling domain and a TCR signaling domain, which can be linked to each other in a random or in a specified order, optionally with a short peptide linker having a length of, for example, between 2 and 10 amino acids (e.g., glycine-serine doublet linker).

In some embodiment, the CAR further comprises a transmembrane domain. When expressed in cells, the anti-TSLP antigen binding domain is extracellular, and the T-cell activation domain is intracellular.

In certain embodiments, the CAR comprises an anti-TSLP antigen binding domain, a transmembrane domain, a costimulatory signaling region, and a TCR signaling domain, wherein the antigen binding domain specifically binds to TSLP and comprises an antigen-binding fragment of the antibodies provided herein.

### 1. Antigen Binding Domain

In some embodiments, the anti-TSLP antigen binding domain of the CAR comprises one or more CDR sequences as provided herein, one or more heavy chain variable domains or light chain variable domains provided herein, or one or more antigen-binding fragment derived from any of the anti-TSLP antibodies provided herein.

In some embodiments, it is beneficial for the antigen binding domain to be derived from the same species in which the CAR will ultimately be used in. For example, for use in humans, it may be beneficial to have the antigen binding domain used in the CAR derived from a human antibody or a humanized antibody. In some embodiments, the antigen binding domain comprises a single chain variable fragment (scFv). In some embodiment, the antigen binding domain may exist in a variety of other forms including, for example, Fv, Fab, and (Fab′)₂, as well as bi-functional (i.e. bi-specific) hybrid antibody fragments (e.g., Lanzavecchia et al., Eur. J. Immunol. 17, 105 (1987)). In certain embodiments, the antigen binding domain comprises a Fab or a scFv.

### 2. Transmembrane Domain

In certain embodiments, the CAR comprises a transmembrane domain fused to the extracellular antigen-binding domain of the CAR. In one embodiment, the transmembrane domain can be selected such that it is naturally associated with one of the domains in the CAR. In some instances, the transmembrane domain can be selected or modified to avoid binding to transmembrane domains of other members of the T cell receptor complex.

The transmembrane domain of the CAR provided herein may be derived from transmembrane domains of any natural membrane-bound or transmembrane protein, such as, for example, the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, and CD154. In some embodiments, the transmembrane domain of the CAR can also use a variety of human hinges such as human Ig (immunoglobulin) hinge.

Alternatively, the transmembrane domain of the CAR provided herein may be synthetic, for example, comprising predominantly hydrophobic residues such as leucine and valine. In one embodiment, a triplet of phenylalanine, tryptophan and valine is included at each end of a synthetic transmembrane domain. Optionally, a short oligo- or polypeptide linker, between 2 and 10 amino acids in length may form the linkage between the transmembrane domain and the intracellular signaling domain of the CAR. A glycine-serine doublet provides a particularly suitable linker.

### 3. TCR Signaling Domain

The T-cell activation domain of the CARs provided herein comprises a TCR signaling domain. The TCR signaling domain can activate the T cell which expresses the CAR, to exert at least one of the normal TCR effector functions of a T cell, for example, cytolytic activity or helper activity including the secretion of cytokines. The TCR signaling domain can be either full-length of a natural intracellular signal transduction domain, or a fragment thereof sufficient to transduce the TCR effector function signal.

Exemplary intracellular signaling domains useful in the CARs provided herein include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

The TCR signaling domain that acts in a stimulatory manner may contain signaling motifs which are known as immunoreceptor tyrosine-based activation motifs or ITAMs. Examples of ITAM containing TCR signaling domains useful in the CAR provided herein include those derived from TCR zeta, FcR gamma, FcR beta, CD3 gamma, CD3 delta, CD3 epsilon, CD5, CD22, CD79a, CD79b, and CD66d. In certain embodiments, the TCR signaling domain comprises a cytoplasmic signaling sequence derived from CD3-zeta.

### 4. Co-Stimulatory Signaling Region

In certain embodiments, the T-cell activation domain of the CARs provided herein may further comprise a co-stimulatory signaling region. Co-stimulatory signaling region acts in an antigen-independent manner to mediate TCR activation and can be derived from a co-stimulatory molecule required for an efficient response of lymphocytes to an antigen. Exemplary co-stimulatory molecules include, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

### 5. Polynucleotide Sequence Encoding the CAR

In one aspect, the present disclosure further provides nucleic acid sequences encoding the CAR provided herein, comprising a first polynucleotide sequence encoding the antigen binding domain of the CAR provided herein, and optionally a second polynucleotide sequence encoding the transmembrane domain and the T-cell activation domain provided herein. In some embodiments, the sequence encoding the antigen binding domain is operably linked to the sequence encoding the transmembrane domain and the T-cell activation domain. The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

In one aspect, the present disclosure provides vectors comprising the nucleic acid sequence encoding the CAR provided herein. In some embodiments, the vector is retroviral and lentiviral vector construct expressing the CAR of the present disclosure which can be directly transduced into a cell, or RNA construct that can be directly transfected into a cell.

In one aspect, the present disclosure provides isolated cells which comprises the nucleic acid sequence encoding the CAR and/or express the CAR provided herein.

In certain embodiments, the cell comprising the nucleic acid encoding the CAR or expressing the CAR is selected from the group consisting of a T cell, a NK cell, a cytotoxic T lymphocyte (CTL), and a regulatory T cell. In one embodiment, the cell comprising the nucleic acid encoding the CAR or expressing the CAR exhibits an antitumor immunity when the antigen binding domain of the CAR binds to its corresponding antigen. The cytotoxic lymphocytes will preferably be autologous cells, although heterologous cells or allogenic cells can be used. As used herein, “autologous” means any material derived from the same individual to whom it is later to be re-introduced into the individual.

In one aspect, the present disclosure further provides methods for stimulating a T cell-mediated immune response to a TSLP-enriched environment or tissue in a subject, the method comprising administering to the subject an effective amount of a cell genetically modified to express the CAR provided herein.

In one aspect, the present disclosure further provides methods for treating a mammal having a disease, disorder or condition associated with an elevated expression of TSLP, comprising administering to the mammal an effective amount of a cell genetically modified to express the CAR provided herein, thereby treating the mammal. In certain embodiments, the cell is an autologous T cell. In certain embodiments, the mammal has been diagnosed with the disease, disorder or condition associated with an elevated expression of TSLP.

### Methods of Use

In another aspect, methods are provided to treat a disease, disorder or condition in a subject that would benefit from modulation of TSLP activity. In another aspect, methods are provided to treat a TSLP related disease or disorder in a subject in need thereof. In another aspect, methods are provided to treat a disease, disorder or condition that is responsive to TSLP inhibition in a subject in need thereof.

In some embodiments, the method comprises administering to the subject a therapeutically effective amount of the antibody or antigen-binding fragment thereof provided herein, or the polynucleotide encoding the antibody or antigen-binding fragment thereof provided herein and/or the pharmaceutical composition provided herein. In certain embodiments, the subject is human.

In some embodiments, the TSLP related disease or disorder is a TSLP-positive disease or disorder. In some embodiments, the subject to be treated has been identified as having a TSLP-positive disease or disorder. In some embodiments, the TSLP related disease, disorder or condition is responsive to TSLP inhibition. In some embodiments, the TSLP related disease, disorder or condition is associated with dysregulation of TSLP mediated signaling, or more specifically, associated with up-regulated TSLP signaling.

In some embodiments, the disease or disorder is associated with cells dysregulation of TSLP mediated signaling. In some embodiments, the dysregulation of TSLP mediated signaling includes dysregulation of STAT5 activation by phosphorylation. In some embodiments, the dysregulation of TSLP mediated signaling includes dysregulation of TSLPR-expressing cell proliferation, compared with control level (e.g., the level in a healthy subject).

In some embodiments, the disease or disorder is an inflammatory disease, an autoimmune disease, or cancer.

In some embodiments, the disease or disorder is selected from the group consisting of: asthma (including severe asthma), idiopathic pulmonary fibrosis, atopic dermatitis (AD), allergic conjunctivitis, allergic rhinitis (AR), Netherton syndrome (NS), eosinophilic esophagitis (EoE), food allergy, allergic diarrhoea, eosinophilic gastroenteritis, allergic bronchopulmonary aspergillosis (ABPA), allergic fungal sinusitis, chronic pruritus, cancer, rheumatoid arthritis, Chronic obstructive pulmonary disease COPD, systemic sclerosis, multiple sclerosis, keloids, ulcerative colitis, chronic rhinosinusitis (CRS), polyposis, chronic eosinophilic pneumonia, eosinophilic bronchitis, allergic bronchopulmonary aspergillosis, coeliac disease, eosinophilic gastroenteritis, ChurgStrauss syndrome, eosinophilic myalgia syndrome, hypereosinophilic syndrome, eosinophilic granulomatosis with polyangiitis, eosinophilic esophagitis, inflammatory bowel disease, fibrotic disorder, inflammatory bowel disease Hodgkin's lymphoma, systemic lupus erythematosus.

In some embodiments, the cancer is selected from: breast cancer, pancreas cancer, colon cancer, lung cancer, ovarian cancer, prostate cancer, and B-cell acute lymphoblastic leukemia.

In some embodiments, the fibrotic disorder is selected from: systemic and local scleroderma, keloids and hypertrophic scars, interstitial lung disease (ILD), idiopathic pulmonary fibrosis (IPF), liver fibrosis resulting from chronic hepatitis B or C infection, radiation-induced fibrosis, and fibrosis arising from wound healing, atherosclerosis, restinosis, pulmonary inflammation and fibrosis, liver cirrhosis, kidney disease, heart disease resulting from scar tissue, and eye diseases such as macular degeneration, and retinal and vitreal retinopathy, fibrosis resulting from chemotherapeutic drugs, and injuries and burns.

In some embodiments, the disease is selected from the group consisting of asthma, Polyp nasal Sinusitis, COPD, urticaria, EoE, and atopic dermatitis.

The presence and/or amount of TSLP in an interested biological sample can be indicative of whether the subject from whom the biological sample is derived could likely respond to an anti-TSLP antibody. Various methods can be used to determine the presence and/or amount of TSLP in a test biological sample from the subject. For example, the test biological sample can be exposed to anti-TSLP antibody or antigen-binding fragment thereof, which binds to and detects the expressed TSLP protein. Alternatively, TSLP can also be detected at nucleic acid expression level, using methods such as qPCR, reverse transcriptase PCR, microarray, serial analysis of gene expression (SAGE), fluorescence in situ hybridization (FISH), and the like. In some embodiments, the test sample is derived from an epithelial tissue. In certain embodiments, presence or up-regulated level of the TSLP in the test biological sample indicates likelihood of responsiveness. The term “up-regulated” as used herein, refers to an overall increase of no less than 10%, 15%, 20%, 25%, 30%, 35%, 40%, 45%, 50%, 55%, 60%, 65%, 70%, 75%, 80% or greater, in the expression level of TSLP in the test sample, as compared to the TSLP expression level in a reference sample as detected using the same method. The reference sample can be a control sample obtained from a healthy or non-diseased individual, or a healthy or non-diseased sample obtained from the same individual from whom the test sample is obtained.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g., physician or veterinarian) as indicated by these and other circumstances or requirements.

In certain embodiments, the antibody or antigen-binding fragment provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies or antigen-binding fragments thereof provided herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the antibodies or antigen-binding fragments thereof provided herein may be administered alone or in combination a therapeutically effective amount of a second therapeutic agent. For example, the antibodies or antigen-binding fragments thereof disclosed herein may be administered in combination with a second therapeutic agent.

The therapeutic agents or drugs useful as the conjugate moiety can be those which are useful for treating asthma, Polyp nasal Sinusitis, chronic obstructive pulmonary disease (COPD), urticaria, eosinophilic esophagitis (EoE), and atopic dermatitis, and the like.

In some embodiments, the second therapeutic agent comprises a therapeutic agent or drug for treating asthma. In some embodiments, the second therapeutic agent comprises beta2-adrenoceptor agonists (SABA, such as salbutamol, salmeterol, formoterol and the like), adrenergic agonists (such as inhaled epinephrine), corticosteroids (preferably inhaled forms such as beclomethasone), leukotriene receptor antagonists (such as montelukast and zafirlukast), mast cell stabilizers (such as cromolyn sodium), macrolide antibiotics, or a combination thereof.

In some embodiments, the second therapeutic agent comprises a therapeutic agent or drug for treating Polyp nasal Sinusitis. In some embodiments, the conjugate moiety comprises antibiotics, kinase inhibitors or steroids

In some embodiments, the second therapeutic agent comprises a therapeutic agent or drug for treating COPD. In some embodiments, the conjugate moiety comprises antibiotics (including amoxicillin, doxycycline and azithromycin), beta2-adrenergic agonists, anticholinergics (such as ipratropium and tiotropium), long-acting muscarinic antagonist (LAMA, such as tiotropium), aclidinium, LAMA umeclidinium bromide, corticosteroids, phosphodiesterase-4 inhibitors (PDE4 inhibitors, such as roflumilast) and the like.

In some embodiments, the second therapeutic agent comprises a therapeutic agent or drug for treating urticaria. In some embodiments, the conjugate moiety comprises antihistamines, systemic steroids, leukotriene-receptor antagonists, anti-inflammatory medications, omalizumab, immunosuppressants, or the like. Non-limiting exemplary antihistamines include diphenhydramine, hydroxyzine, loratadine, cetirizine, desloratadine, fexofenadine and the like.

In some embodiments, the second therapeutic agent comprises a therapeutic agent or drug for treating EoE. In some embodiments, the second therapeutic agent comprises proton-pump inhibitors (PPIs), corticosteroids, and the like.

In some embodiments, the second therapeutic agent comprises a therapeutic agent or drug for treating atopic dermatitis. In some embodiments, the conjugate moiety comprises corticosteroids (such as hydrocortisone), calcineurin inhibitors (such as tacrolimus or pimecrolimus), tacrolimus, pimecrolimus, PDE-4 inhibitor (such as crisaborole), systemic immunosuppressants (such as ciclosporin, methotrexate, interferon gamma-1b, mycophenolate mofetil, and azathioprine), additional antibodies (such as dupilumab, tralokinumab), and the like.

In certain of these embodiments, an antibody or antigen-binding fragment thereof provided herein that is administered in combination with one or more additional therapeutic agents may be administered simultaneously with the one or more additional therapeutic agents, and in certain of these embodiments the antibody or antigen-binding fragment thereof and the additional therapeutic agent(s) may be administered as part of the same pharmaceutical composition. However, an antibody or antigen-binding fragment thereof administered “in combination” with another therapeutic agent does not have to be administered simultaneously with or in the same composition as the agent. An antibody or antigen-binding fragment thereof administered prior to or after another agent is considered to be administered “in combination” with that agent as the phrase is used herein, even if the antibody or antigen-binding fragment and the second agent are administered via different routes. Where possible, additional therapeutic agents administered in combination with the antibodies or antigen-binding fragments thereof disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57^{th} edition (November 2002)) or protocols well known in the art.

In another aspect, the present disclosure further provides methods of modulating TSLP activity, comprising exposing the TSLP and TSLPR-positive cells to the antibodies or antigen-binding fragments thereof provided herein.

In another aspect, the present disclosure provides methods of detecting the presence or amount of TSLP in a sample, comprising contacting the sample with the antibody or antigen-binding fragment thereof provided herein, and determining the presence or the amount of TSLP in the sample.

In another aspect, the present disclosure provides a method of diagnosing a TSLP related disease, disorder or condition in a subject, comprising a) contacting a sample obtained from the subject with the antibody or an antigen-binding fragment thereof provided herein; b) determining the presence or amount of TSLP in the sample; and c) correlating the presence or the amount of TSLP to existence or status of the TSLP related disease, disorder or condition in the subject.

In another aspect, the present disclosure provides kits comprising the antibody or antigen-binding fragment thereof provided herein, optionally conjugated with a detectable moiety, which is useful in detecting a TSLP related disease, disorder or condition. The kits may further comprise instructions for use.

In another aspect, the present disclosure also provides use of the antibody or antigen-binding fragment thereof provided herein in the manufacture of a medicament for treating, preventing or alleviating a TSLP related disease, disorder or condition in a subject, in the manufacture of a diagnostic reagent for diagnosing a TSLP related disease, disorder or condition.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. TSLPR-hIL7Ra Protein Expression

Human IL7RA was obtained from human IL-7R (NM_002185) cDNA clone (Fenghui biological G120380), and human TSLPR_23-231 gene fragment was obtained from human CRLF2 (NM_022148) cDNA clone (Fenghui biological G157739). The two gene fragments were connected by a synthetic GGS linker and constructed into the vector Ori. Vec.E016-pTT5-hIgG1.CH1.His. The IgG light chain signal peptide sequence was added at the N terminal and 10× His peptide at the C terminal in the sequence. The target vector huIL7Ra-huTSLPR His fusion was used to obtain pure receptor complexes. After the constructed vector was verified by sequencing, the correct clone was extracted for plasmid extraction. The transfection level plasmid was prepared and transfected into freestyle 293T cells. Supernatant was collected on the 7th day, then affinity purified by nickel column, and the protein was replaced into 20 mm PB and 150 mm NaCl buffer by dialysis. SDS-PAGE electrophoresis and ELISA binding assay results showed that the purity of the protein was more than 90% and the binding activity was qualified. The purified TSLPR-hIL7Ra protein can be used for later functional screening.

### Example 2. Cell Line Establishment

In order to develop monoclonal antibodies that bind and block the function of TSLP, we constructed the following cell lines. All constructed stable overexpression cell lines were packaged with lentivirus and infected with related cell lines.

### 2.1 Membrane Long-Form and Short-Form Human TSLP Expressing Cells

The long-form (29-159) and short-form (97-159) human TSLP (huTSLP) were obtained from the synthetic gene fragment SD.hTSLP (NP_149024.1, R127A; R130S), then constructed into the vector Ori.Vec.E055-pCDH.CMV-Sig.CD3TM-EF1.GFP.T2A.Puro-RE to obtain two overexpression vectors pCDH.CMV-Sig.CD3TM-EF1.GFP.T2A.Puro-RE-hLTSLP, pCDH.CMV-Sig.CD3TM-EF1.GFP.T2A.Puro-RE-hSTSLP, which display TSLP protein on the cell membrane through the transmembrane region of CD3. The amino acid sequence of the membrane long-form huTSLP fusion protein is shown as SEQ ID NO: 80. The amino acid sequence of the membrane short-form huTSLP fusion protein is shown as SEQ ID NO: 79. After the plasmid was sequenced to verify the correct expression frame, the overexpression cell line of related cells was constructed by packaging lentivirus and infecting HEK-293 cells.

The short-form TSLP was comprised in a membrane short-form TSLP fusion protein, which was expressed on the cell membrane. The membrane short-form TSLP fusion protein further comprised a signal peptide at the N-terminal of the short-form TSLP sequence and a transmembrane region at the C-terminal of the short-form TSLP sequence, with an amino acid sequence as shown in SEQ ID NO: 79.

Amino acid sequence of membrane short-form TSLP fusion protein (SEQ ID NO: 79):

MALPVTALLLPLALLLHAARPMFAMKTKAALAIWCPGYSETQINAT
QAMKKARKSKVTTNKCLEQVSQLQGLWRRFNRPLLKQQTTTPAPRPPTP
APTIASQPLSLRPEACRPAAGGAVHTRGLDFACDIYIWAPLAGTCGVLL
LSLVITLYCKRGRKKLLYIFKQPFMRPVQTTQEEDGCSCRFPEEEEGGC
ELRVKFSRSADAPAYKQGQNQLYNELNLGRREEYDVLDKRRGRDPEMGG
KPRRKNPQEGLYNELQKDKMAEAYSEIGMKGERRRGKGHDGLYQGLSTA
TKDTYDALHMQALPPR

The long-form TSLP was comprised in a membrane long-form TSLP fusion protein, which was expressed on the cell membrane. The membrane long-form TSLP fusion protein further comprised a signal peptide at the N-terminal of the long-form TSLP sequence and a transmembrane region at the C-terminal of the long-form TSLP sequence, with an amino acid sequence as shown in SEQ ID NO: 80.

Amino acid sequence of membrane long-form TSLP fusion protein (SEQ ID NO: 80):

MALPVTALLLPLALLLHAARPYDFTNCDFEKIKAAYLSTISKDLITYM
SGTKSTEFNNTVSCSNRPHCLTEIQSLTFNPTAGCASLAKEMFAMKTKA
ALAIWCPGYSETQINATQAMKKARKSKVTTNKCLEQVSQLQGLWRRFNR
PLLKQQTTTPAPRPPTPAPTIASQPLSLRPEACRPAAGGAVHTRGLDFA
CDIYIWAPLAGTCGVLLLSLVITLYCKRGRKKLLYIFKQPFMRPVQTTQ
EEDGCSCRFPEEEEGGCELRVKFSRSADAPAYKQGQNQLYNELNLGRRE
EYDVLDKRRGRDPEMGGKPRRKNPQEGLYNELQKDKMAEAYSEIGMKGE
RRRGKGHDGLYQGLSTATKDTYDALHMQALPPR

### 2.2 Membrane CyTSLP Expressing Cell

According to the amino acid and nucleotide sequence information of cynomolgus monkey TSLP (XM_00557498.2) on NCBI, the gene CyTSLP was synthesized and constructed into the vector Ori.Vec.E055-pCDH.CMV-Sig.CD3TM-EF1.GFP.T2A.Puro-RE, and the overexpression vector pCDH.CMV-Sig.CD3TM-EF1.GFP.T2A.Puro-RE-CynoTSLP was obtained, which display the CyTSLP protein on the cell membrane through the transmembrane region of CD3. The amino acid sequence of the membrane CyTSLP fusion protein is shown in SEQ ID NO: 81. After the plasmid was sequenced to verify the correct expression frame, the overexpression cell lines of related cells were constructed by packaging lentivirus and infecting HEK-293 and Ba/F3 cells.

Amino acid sequence of the membrane CyTSLP fusion protein (SEQ ID NO: 81):

MALPVTALLLPLALLLHAARPYDFTNCDFQKIEADYLRTISKDLITYM
SGTKSTDFNNTVSCSNRPHCLTEIQSLTFNPTPRCASLAKEMFARKTKA
TLALWCPGYSETQINATQAMKKRRKRKVTTNKCLEQVSQLLGLWRRFIR
TLLKKQTTTPAPRPPTPAPTIASQPLSLRPEACRPAAGGAVHTRGLDFA
CDIYIWAPLAGTCGVLLLSLVITLYCKRGRKKLLYIFKQPFMRPVQTTQ
EEDGCSCRFPEEEEGGCELRVKFSRSADAPAYKQGQNQLYNELNLGRRE
EYDVLDKRRGRDPEMGGKPRRKNPQEGLYNELQKDKMAEAYSEIGMKGE
RRRGKGHDGLYQGLSTATKDTYDALHMQALPPR

2.3 Membrane huTSLPR-huIL7Ra Expressing Cell

The full length of human IL7RA gene fragment and human TSLPR gene fragment were obtained from human IL7R (NM_002185) cDNA clone (Fenghui biological G120380) and human CRLF2 (nm_022148) cDNA clone (Fenghui biological G157739). They were constructed into vector pL VX-IRES-Neo and vector Ori.Vec.E025-pCDH-CMV.MCS-EF1.CopGFP.T2A.Puro respectively by homologous recombination, two overexpression vectors human IL7Ra-pLVX-IRES-Neo and human TSLPR-pCDH-CMV-Puro were obtained. The amino acid sequence of human IL7RA protein is shown as SEQ ID NO: 71, respectively. The amino acid sequence of human TSLPR protein is shown as SEQ ID NO: 72. After the plasmid was sequenced to verify the correct expression frame, the overexpression cell lines of related cells were constructed by packaging lentivirus and infecting HEK-293 and Ba/F3 cells.

2.4 293T Cell Expressing hTSLPR-hIL7Ra and STAT5-Luciferase

STAT5 luciferase virus was used to infect 293T-hTSLPR-hIL7Ra stable cells. After 24 hours, the fresh culture medium was changed and 100 ug/ml hygro was added for screening. After 8 days of screening, the positive rate of FACS was detected. The positive rate of expressed cells was more than 90%. The expression of luciferase could be detected after stimulation with hTSLP, indicating that the receptor expressed by the cell has the function of activating STAT5 luciferase.

### Example 3. Immunization

In order to obtain antibodies specifically binding to long form TSLP, 6-8 weeks SPF Balb/C female healthy mice (purchased from Zhejiang Vital River Experimental Animal Technology Co., Ltd.) were selected. The mice were immunized for several times with recombinant human long form TSLP antigen (purchased from ACRO, TSP-H52Ha, having an amino acid sequence of SEQ ID NO: 73, the mutated long form TSLP with R127A and R130A mutations) and equal volume of Freund's adjuvant, for the first time using Freund's complete adjuvant, and for strengthen using Freund's incomplete adjuvant. From the second booster immunization, the orbital blood of mice was collected within 6-8 days after each booster immunization, and the immune serum titer of mice was detected by indirect ELISA. The results show that mice can produce high titer antibodies specific for human long form TSLP after immunization, indicating that the scheme can meet the preparation of subsequent hybridoma antibodies.

### Example 4. Hybridoma Generation and Screening

Mice with stable and qualified serum titer after two booster immunizations were selected for cell fusion. The spleen lymphocytes of these mice were electro-fused with SP2/0 myeloma cells. The fusion clones were screened by hypoxanthine aminopterin thymidine (HAT) and cultured for 7 days. The cell supernatant was detected by indirect ELISA (same as methods described below in Section 4.1 of this Example).

Flow cytometry (same as method described below in Section 4.2 of this Example) was used to detect the blocking activity of the supernatant on the binding of human long-form TSLP (His Tag, TSP-H52Ha), monkey long-form TSLP (His Tag, TSP-C52H8) (all purchased from ACRO) to 293T-hTSLP/hIL7Ra cells.

According to the results of ELISA and flow cytometry, the clones with good ELISA binding and FACS blocking activity were selected, and 2-3 rounds of subclones were carried out by limited dilution method. When the subclonal culture were all ELISA positive and at least two monoclonal culture were flow cytometry positive, two clones were selected from each hybridoma for strain identification and expanded culture.

After subclonal screening, 98 human TSLP specific hybridoma cell lines were finally obtained for subsequent development.

Through the analysis of heavy and light chain sequence of hybridoma antibody, 69 hybridoma cells with different VH sequences were cultured in serum-free medium to express hybridoma antibody. The culture supernatant was collected, protein G affinity purified, and the antibody was replaced into 20 mM PB and 150 mM NaCl buffer by dialysis. Through SDS-PAGE electrophoresis and binding verification, the purity of the antibody was >90%, and the binding activity was qualified, which can be used for antibody screening in the later stage.

### 4.1 ELISA Binding Test

1 ug/ml human long-form TSLP protein (Acro, tsp-h5255 or tsp-h52ha) or cynomolgus long-form TSLP protein (Acro, tsp-c52h8) antigen solution was prepared with PBS, coated in 384 well plates with 25 ul per well overnight at 4° C. The antigen coating solution was discarded. 80 ul/well 3% milk was added and sealed at room temperature for 1.5 hours. The supernatant was discarded, and the plate was washed with PBST (0.1% T20) for 3 times. 25 ul of samples were added to 384 well plate for incubation for 1 hour at room temperature. The supernatant was discarded, and the plate was washed with PBST (0.1% T20) for 3 times. 25 ul secondary antibody was added to 384 well plate per well for incubation at room temperature for 1 hour. The supernatant was discarded, and the plate was washed with PBST (0.1% T20) for 7 times. HRP substrate TMB was added, 25 ul/well. After incubation for 12 minutes, 25 ul termination solution and 2M HCl were added to terminate the reaction. The microplate reader was used to read the absorbance value of 450 nm. Antibodies showing specific binding to human long-form TSLP were identified and selected.

### 4.2 FACS Blocking Test

The antibody with cell culture medium was diluted and transferred with 50 ul to a new 96 well plate per well. 10 ul of 0.012 ug/ml human long-form TSLP protein (Acro, tsp-h52ha) or cynomolgus long-form TSLP protein (Acro, tsp-c52h8) was added per well, for incubation at 4° C. for 0.5h. 293T-TSLPR-IL7R stable cells (constructed according to section 2.3 of Example 2) were added to 96 well plate, and the supernatant was discarded after centrifugation. The pre-incubated antibody and TSLP were transferred to cell plate for 50 ul, mixed, and incubated at 4° C. for 0.5 hours. After centrifugation, the supernatant was washed with PBS for 3 times, and the supernatant was discarded after centrifugation. 50 ul secondary antibody was added to 96 well plate, for incubation at 4° C. for 0.5 hours. The supernatant was washed with PBS for 3 times after centrifugation, and the supernatant was discarded after centrifugation. The cells were resuspended with 100 ul PBS, and the fluorescence values were red by flow cytometry. Antibodies showing specific blockade to binding of long form human TSLP to 293T-TSLPR-IL7R stable cells were identified and selected.

### 4.3 Membrane CyTSLP-Expressing Cell Binding Test

CyTSLP expressing cells (constructed according to section 2.2 of Example 2) were added to 96 well plate, and supernatant was discarded after centrifugation. 50 ul of the antibody solution diluted with D10 (DMEM+10% FBS) was transferred to cell plate, for incubation at 4° C. for 0.5 hours. The plate was washed with PBS after centrifugation for 3 times, and the supernatant was discarded after centrifugation. 50 ul secondary antibody was added to 96 well plate, for binding at 4° C. for 0.5 hours. The supernatant was removed after centrifugation, and the plate was washed with PBS for 3 times, and the supernatant was discarded after centrifugation. The cells were resuspended with 100 ul PBS and the fluorescence values were read by flow cytometry.

### 4.4 TSLP Dependent BaF3 Proliferation Test

After BaF3-hTSLPR-hIL7R cells were thawed, they were washed twice with hybridoma serum-free medium, diluted to 4E5/ml, and added 30 ul per well into 384 well cell culture plate. Hybridoma serum-free medium was used to prepare 20 ng/ml human long form TSLP protein (Acro, tsp-h52ha), and 15 ul per well was added to the cell culture plate. The antibody was prepared with hybridoma serum-free medium, and added 15 ul per well to the cell culture plate, for incubation in 37° C. and 5% CO₂ cell culture box for 72 hours. The 384 well cell culture plate was taken out and added with 15 ul CTG (Promega, g7572). The plate was shaken for 5 min after centrifugation, and centrifuged again, and the luminescence values were red within 30 min. Antibodies showing specific inhibition on recombinant long form TSLP induced BaF3 proliferation were identified and selected.

Through ELISA, FACS blocking test, and function test as described in Examples 4.1, 4.2 and 4.4, clones with good activity were selected for chimeric antibody preparation. These candidate sequences were further verified and screened by chimeric expressed antibodies.

### Example 5. Chimeric Antibody Generation and Screening

According to the hybridoma antibody screening result, we constructed the recombinant expression vector and purified the selected 15 antibodies. The process was as follows: using the heavy chain sequencing plasmid obtained by hybridoma sequencing as the template, the heavy chain variable region fragment was amplified by PCR and cloned into the pTT5-hIgG2.CH vector containing the amino acid sequence of IgG2 heavy chain constant region by homologous recombination to obtain the chimeric antibody heavy chain expression vector. Similarly, using the light chain sequencing plasmid obtained by hybridoma sequencing as the template, the light chain variable region fragment was amplified by PCR and cloned into the pTT5-hKappa.CL vector, which has amino acid sequence of light chain constant region CL of κ light chain, to obtain the chimeric antibody light chain expression vector.

The constructed transient expression vector was used to prepare 50-100 ug endotoxin free plasmid, which was transfected into freestyle 293T cells, affinity purified by protein A, and the antibody was replaced into 20 mM PB and 150 mM NaCl buffer by dialysis. Among them, two clones were not obtained due to their low expression. After SDS-PAGE electrophoresis and binding verification, the purity of the other 13 antibodies was >90%, and the binding activity was qualified, which can be used for antibody screening in the later stage.

Then, two clones 39H11 and 35G12 with good activity were screened for humanization through FACS blocking test and function test, following the same methods as described in Example 4.

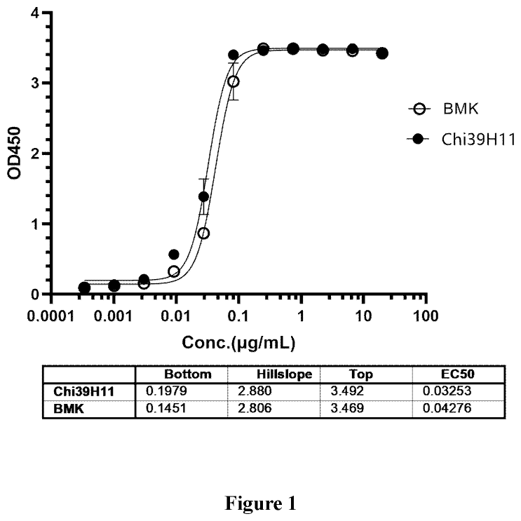

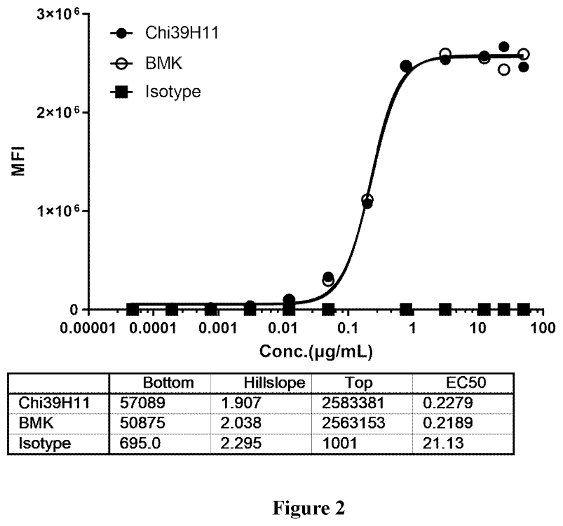

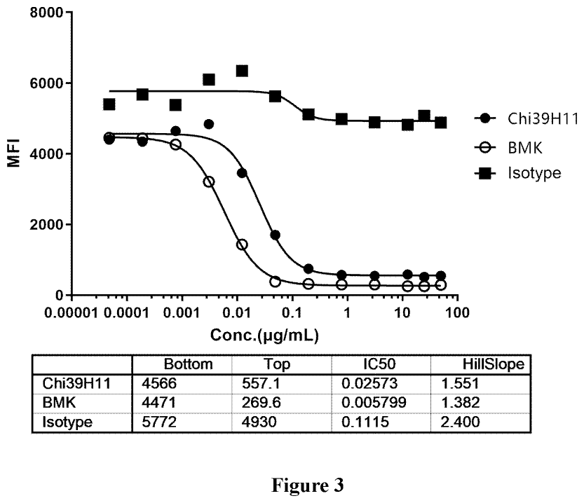

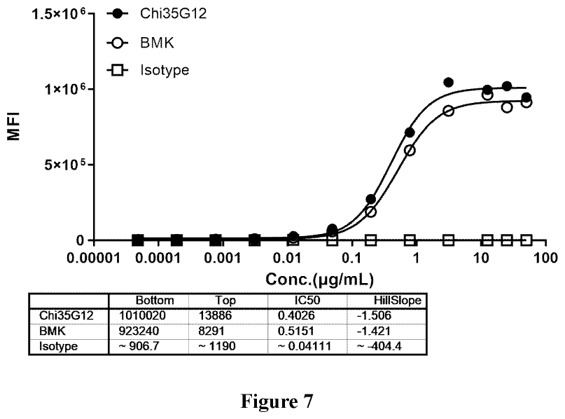

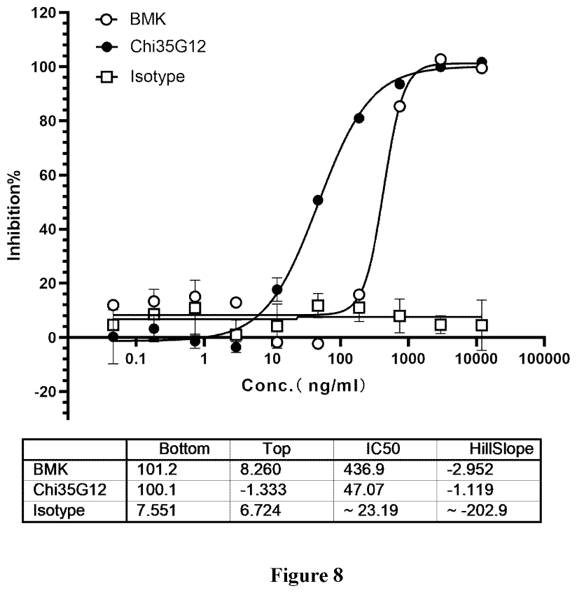

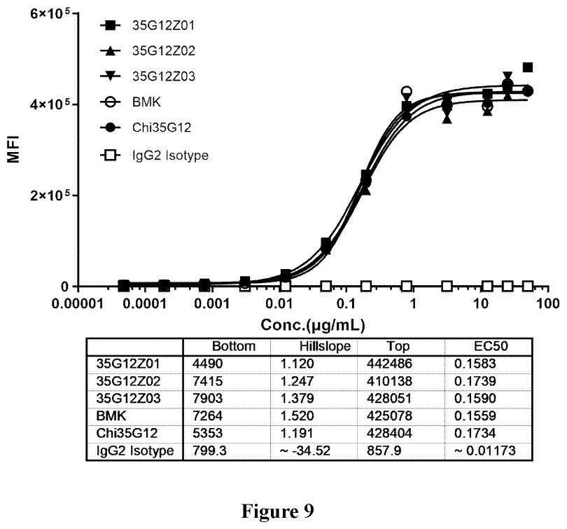

The 39H11 chimeric antibody was named Chi39H11 and the 35G12 chimeric antibody was named Chi35G12. The results of binding test (FIG. 1, FIG. 2, and FIG. 7), blocking test (FIG. 3) and function test (FIG. 8) are shown.

### Example 6. Humanization and Lead Generation

### 6.1 39H11

The candidate antibodies were humanized according to the commonly known CDR transplantation method. Use IMGT/V-QUEST tool (http://www.imgt.org/IMGT_vquest/input), the sequences of antibody heavy chain variable region (VH) and light chain variable region (VL) were analyzed to determine the CDR region sequences of antibody light chain and heavy chain. Using the IgBlast tool (https://www.ncbi.nlm.nih.gov/igblast/), the amino acid sequence of the antibody was analyzed to obtain the closest VH and VL sequences of human race. The CDR regions of the antibody were grafted into the frame regions of the selected VH and VL sequences, which were the humanized antibody sequences.

Then, the back mutation was carried out for individual amino acids in the antibody sequence. Homo sapiens codon optimization and gene synthesis were carried out, the heavy chain genes were constructed into pTT5-hIgG2.CH vector containing amino acid sequence of IgG2 heavy chain constant region, light chain genes were constructed into pTT5-hKappa.CL vector containing amino acid CL sequence of antibody κ light chain constant region. The constructed transient expression vectors were paired with light and heavy chains and transfected into freestyle 293T cells. The antibodies were expressed and purified, then was taken for quantification, binding, blocking and function evaluation. Two humanized antibody clones 39H11Z07 and 39H11Z11 were selected for further evaluation.

Back mutation to mouse sequence was introduced to the corresponding sites of the humanized sequence. Mutant vectors obtained, paired and transfected into freestyle 293T cells. Antibodies were expressed and purified, and tested for binding, blocking and function evaluation.

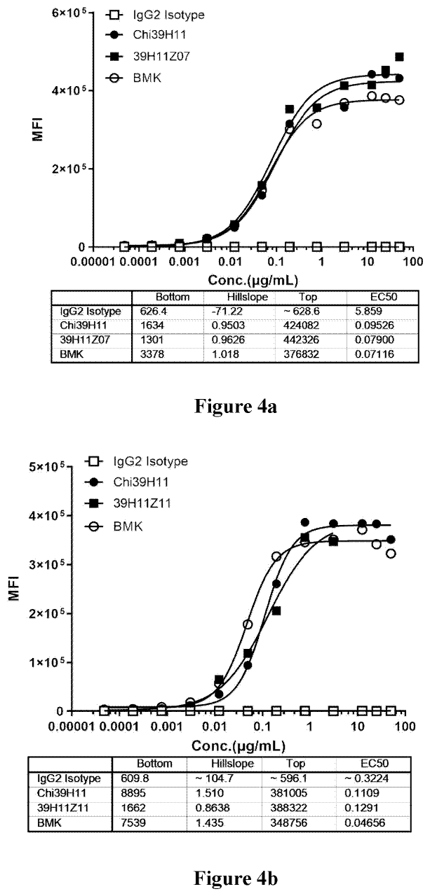

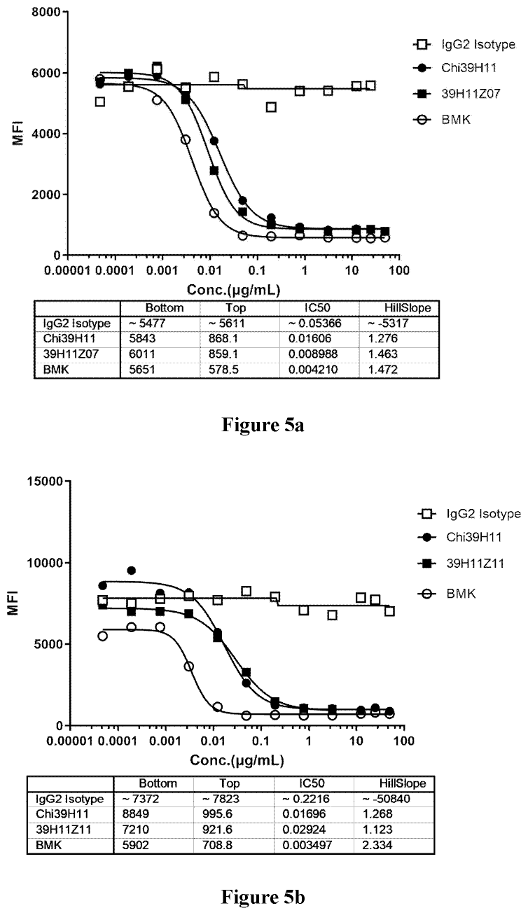

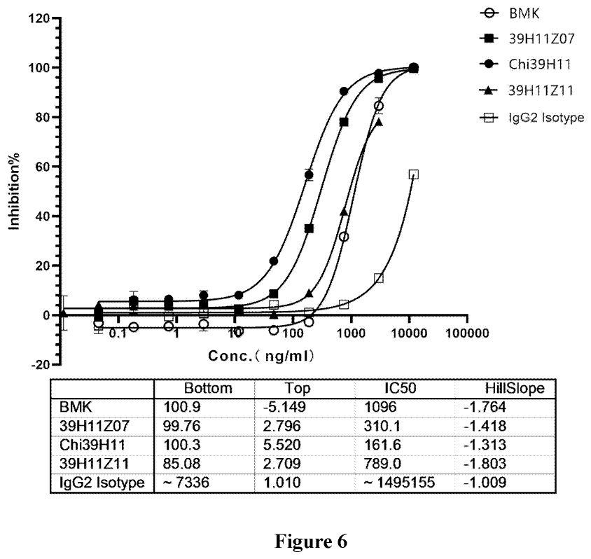

Results were shown in FIGS. 4a, 4b, 5a, 5b and 6. The results show that the blocking activity of 39H11Z07 and 39H11Z11 is comparable to Chi39H11 (FIG. 5a, 5b), and function better than BMK (FIG. 6).

### 6.2 35G12

The candidate antibodies were humanized according to the commonly known CDR transplantation method. Use IMGT/V-QUEST tool (http://www.imgt.org/IMGT_vquest/input), the sequences of antibody heavy chain variable region (VH) and light chain variable region (VL) were analyzed to determine the CDR region sequences of antibody light chain and heavy chain. Using the IgBlast tool (https://www.ncbi.nlm.nih.gov/igblast/), the amino acid sequence of the antibody was analyzed to obtain the closest VH and VL sequences of human race. The CDR regions of the antibody were grafted into the frame regions of the selected VH and VL sequences, which were the humanized antibody sequences.

Then, the back mutation was carried out for individual amino acids in the antibody sequence. Homo sapiens codon optimization and gene synthesis were carried out, the heavy chain genes were constructed into pTT5-hIgG2.CH vector containing amino acid sequence of IgG2 heavy chain constant region, light chain genes were constructed into pTT5-hKappa.CL vector containing amino acid CL sequence of antibody κ light chain constant region. The constructed transient expression vectors were paired with light and heavy chains and transfected into freestyle 293T cells. The antibodies were expressed and purified, then was taken for quantification, binding, blocking and function evaluation.

Back mutation to mouse sequence was introduced to the corresponding sites of the humanized sequence. Mutant vectors obtained, paired and transfected into freestyle 293T cells. Antibodies were expressed and purified, and tested for binding, blocking and function evaluation. Three humanized antibody clones 35G12Z01, 35G12Z02 and 35G12Z03 were selected for further evaluation.

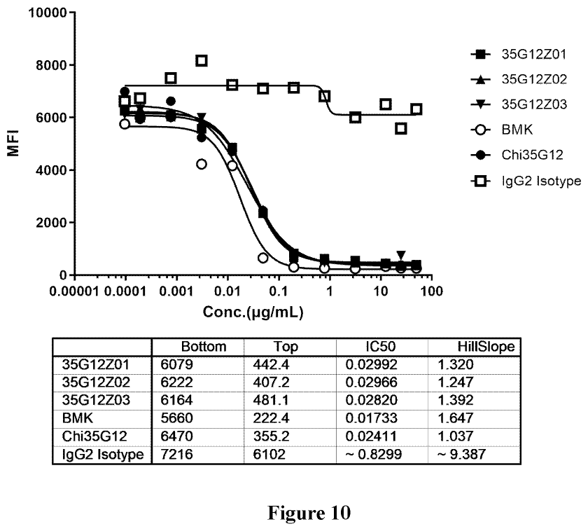

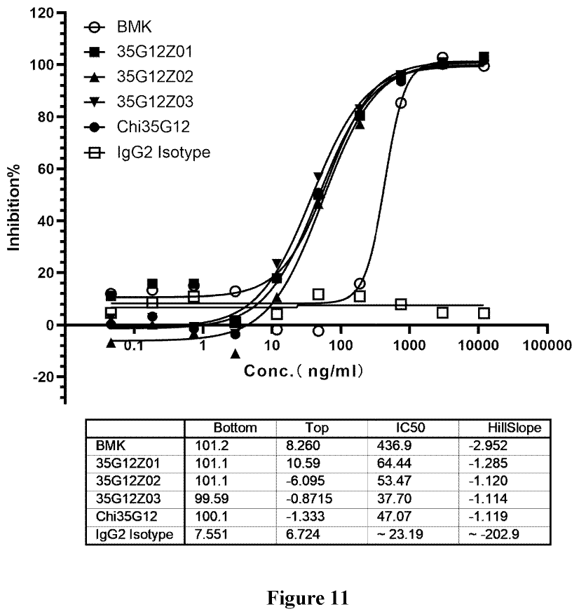

Then, the expression and purification of the antibodies were carried out according to the preparation process of chimeric antibody. The obtained antibodies were tested for binding activity, blocking activity and function. The results are shown in FIG. 9 to FIG. 11. The results show that the inhibitory effect of 35G12Z01, 35G12Z02 and 35G12Z03 on the human long-form TSLP dependent proliferation of BaF3 cells is better than BMK and Chi35G12 (FIG. 11).

### Example 7. In Vitro Characterization

The two screened antibodies 39H11Z07 and 35G12Z03 were modified by Fc with reduced ADCC and prolonged half-life.

S228P\F234A\L235A\M252Y\S254T\T256E\K447del mutations were introduced to obtain IgG4-PAA-YTE heavy chain constant region expression vector. The amino acid sequence of IgG4-PAA-YTE heavy chain constant region is shown as SEQ ID NO: 75.

Amino acid sequence of constant region of human IgG4-PAA-YTE heavy chain (SEQ ID NO: 75):

ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEAAGGPSVFLFPPKPKDTLYITREPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLG

L234A\L235A\M252Y\S254T\T256E mutations were introduced to obtain IgG1-LALA-YTE heavy chain constant region expression vector. The amino acid sequence of IgG1-LALA-YTE heavy chain constant region is shown as SEQ ID NO: 88. Amino acid sequence of the heavy chain constant region IgG1-LALA-YTE Fc (SEQ ID NO: 88):

ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLYITREPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

39H11Z07 heavy chain variable region fragment was amplified by PCR and cloned into pTT5HE-hIgG4CH-PAA-YTE-RE vector containing IgG4-PAA-YTE heavy chain constant region amino acid sequence by homologous recombination, and 39H11Z07 Fc replaced expression vector was obtained. Similarly, 35G12Z03 heavy chain variable region fragment was amplified by PCR with h35G12.H3 plasmid as template and cloned into pTT5HE-hIgG4CH-PAA-YTE-RE vector containing IgG4-PAA-YTE heavy chain constant region amino acid sequence by homologous recombination.

The 35G12Z03 Fc replaced expression vector were obtained. The constructed transient expression heavy chain vector and its corresponding light chain vector were used to prepare endotoxin free plasmid, which was transfected into freestyle 293T cells, affinity purified by protein A, and the antibody was replaced into 20 mm PB and 150 mM NaCl buffer by dialysis or cation chromatography.

In the examples, the benchmark (BMK in short) used is AMG 157, with a heavy chain comprising an amino acid sequence of SEQ ID NO: 77 and a light chain comprising an amino acid sequence of SEQ ID NO: 78.

Amino acid sequence of AMG 157 heavy chain (SEQ ID NO: 77):

QMQLVESGGGVVQPGRSLRLSCAASGFTFRTYGMHWVRQAPGKGL
EWVAVIWYDGSNKHYADSVKGRFTITRDNSKNTLNLQMNSLRAEDTAVY
YCARAPQWELVHEAFDIWGQGTMVTVSSASTKGPSVFPLAPCSRSTSES
TAALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPSV
FLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISK
TKGQPREPQVYTLPPSREEMTKNQVSLTCLVKGFYPSDIAVEWESNGQP
ENNYKTTPPMLDSDGSFFLYSKLTVDKSRWQQGNVFSCSVMHEALHNHY
TQKSLSLSPGK
Amino acid sequence of AMG 157 light chain (SEQ
ID NO: 78):
SYVLTQPPSVSVAPGQTARITCGGNNLGSKSVHWYQQKPGQAPVLV
VYDDSDRPSWIPERFSGSNSGNTATLTISRGEAGDEADYYCQVWDSSSD
HVVFGGGTKLTVLGQPKAAPSVTLFPPSSEELQANKATLVCLISDFYPG
AVTVAWKADSSPVKAGVETTTPSKQSNNKYAASSYLSLTPEQWKSHRSY
SCQVTHEGSTVEKTVAPTECS

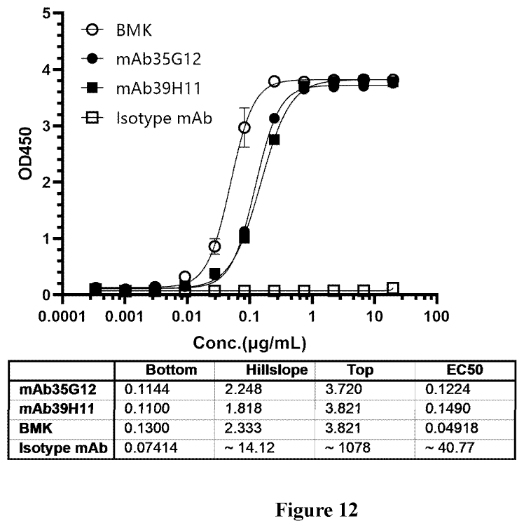

The obtained antibodies were characterized for binding, blocking and functional test, the results of which are as shown in FIG. 12 to FIG. 16. The results show that different Fc antibodies have little difference in binding activity, blocking activity and function, which are better than BMK.

### 7.1 ELISA Binding

1 ug/ml human long-form TSLP protein (Acro, tsp-h5255 or tsp-h52ha) or cynomolgus long-form TSLP protein (Acro, tsp-c52h8) antigen solution was prepared with PBS, coated in 384 well plates with 25 ul per well overnight at 4° C. The antigen coating solution was discarded. 80 ul/well 3% milk was added and sealed at room temperature for 1.5 hours. The supernatant was discarded, and the plate was washed with PBST (0.1% T20) for 3 times. 25 ul of samples were added to 384 well plate for incubation for 1 hour at room temperature. The supernatant was discarded, and the plate was washed with PBST (0.1% T20) for 3 times. 25 ul secondary antibody was added to 384 well plate per well for incubation at room temperature for 1 hour. The supernatant was discarded, and the plate was washed with PBST (0.1% T20) for 7 times. HRP substrate TMB was added, 25 ul/well. After incubation for 12 minutes, 25 ul termination solution and 2M HCl were added to terminate the reaction. The microplate reader was used to read the absorbance value of 450 nm.

The results are shown in FIG. 12, which showed that antibodies mAb35G12 and mAb39H11 in present disclosure can bind to the TSLP protein.

### 7.2 Affinity Determination

The affinity of the antibody was determined using the probes of Gator and HFC (Gator, pl168-160003). The five steps, including balance, loading, balance, association, and dissociation, were adopted.

Different types of antibodies were diluted to the concentration of 10 ug/ml for loading, and then the sensor was balanced with human long form TSLP protein (Acro, tsp-h5255 or tsp-h52ha) with the same concentration of 50 nm, 25 nm, 12.5 nm, 6.25 nm, 3.12 nm, 1.56 nm and 0 concentration reference respectively. The Kon values were tested. After binding to equilibrium, the dissociation curves of antibody antigen complex in the dissociation buffer were determined and the koff values were calculated.

The Kd values of candidate molecules are as follows. The results show that the Kd values of different Fc forms of candidate clones mAb35G12 and mAb39H11 combined with antigen are equivalent to that of AMG157 antibody.

TABLE 6
Affinity determination using Gator

| Sample | Koff(1/s) | Kon(1/Ms) | KD(M) | FullR² |
|---|---|---|---|---|
| mAb35G12 | 2.28E−03 | 1.15E+06 | 1.98E−09 | 0.958 |
| mAb39H11 | 3.31E−03 | 1.58E+06 | 2.10E−09 | 0.908 |
| BMK | 1.69E−03 | 2.13E+06 | 7.49E−10 | 0.971 |

### 7.3 Binding to Different Species TSLP

In order to detect the binding of antibodies to different species of TSLP, 1 ug/ml human long-form TSLP protein (ACRO, tsp-h5255 or tsp-h52ha), cynomolgus long-form TSLP protein (ACRO, tsp-c52h8), mouse long-form TSLP protein (ACRO, tsp-m52h8) and rat long-form TSLP protein (ACRO, tsp-r52h3) antigen solution were prepared with PBS. Each well was coated with 384 well plate at 25 ul and coated overnight at 4° C. After that, the supernatant was discarded and 80 ul/well 3% milk was added and sealed at room temperature for 1.5 hours. The supernatant was discarded and the plate was washed with PBST (0.1% T20) for 3 times. 25 ul of samples to be tested were added to 384 well plate for incubation for 1 hour at room temperature. The supernatant was discarded and the plate was washed with PBST (0.1% T20) for 3 times. 25 ul secondary antibody was added to 384 well plate per well for incubation at room temperature for 1 hour. The supernatant was discarded and the plate was washed with PBST (0.1% T20) for 7 times. HRP substrate TMB was added, 25 ul/well. After 12 minutes, 25 ul termination solution and 2m HCl were added to terminate the reaction. The microplate reader was used to read the absorbance value of 450 nm.

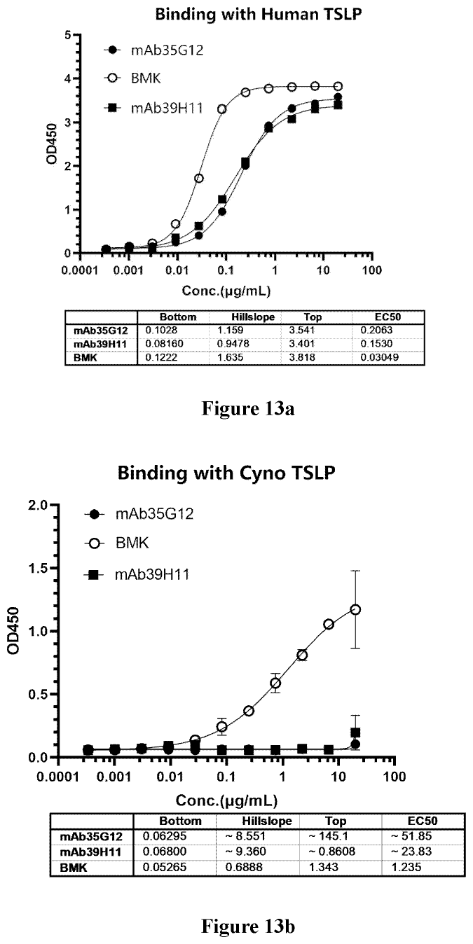

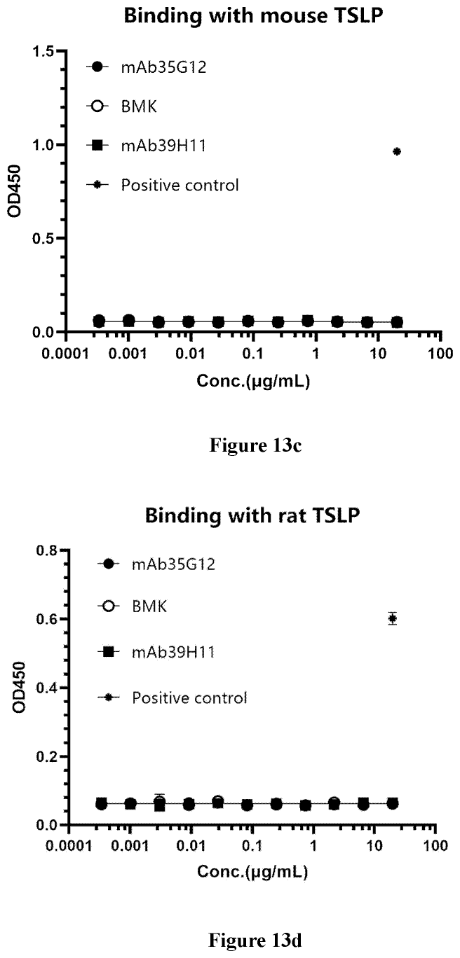

The results were shown in FIGS. 13a to 13d. The results of ELISA show that all the molecules of mAb35G12 and mAb39H11 can bind human long-form TSLP, and none of the molecules can bind monkey, rat and mouse long-form TSLP, except the BMK can bind to monkey long-form TSLP with low potency.

### 7.4 Epitope Analysis

The epitope of the antibody was determined using Gator and His probe (Gator, pl168-160009).

After the probe was balanced, the human long-form TSLP protein (Acro, tsp-h52ha) antigen with a concentration of 5 ug/ml was loaded. The first antibody of 10 ug/ml was loaded after balancing, and the second antibody of 10 ug/ml was loaded after re-balancing. Finally, the binding thickness (nm) of the second antibody was obtained, and this parameter was used for analyzing whether the epitopes are the same.

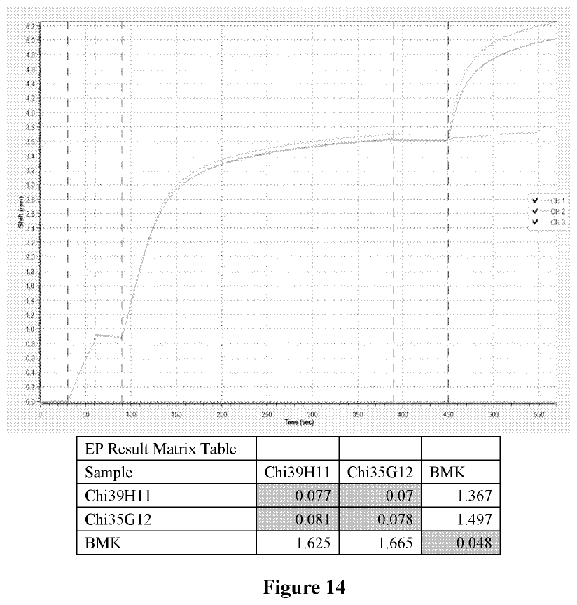

The results were shown in FIG. 14, which show that binding epitopes of Chi39H11 and Chi35G12 are different from that of BMK.

### 7.5 Receptor Blocking

The protocols of receptor blocking test for the humanized antibodies were the same as those described in Example 4.1.

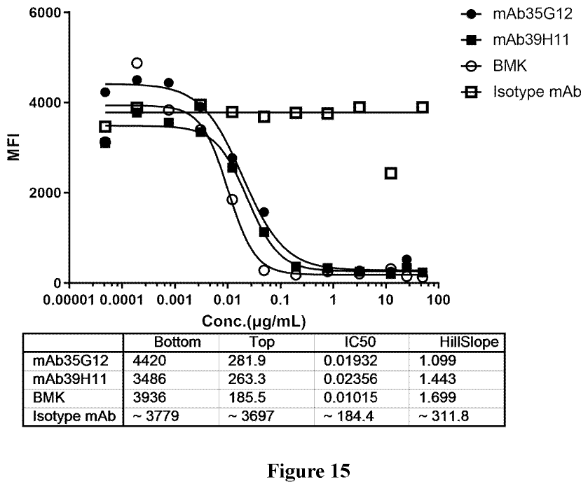

The results were shown in FIG. 15, which show that antibodies mAb35G12 and mAb39H11 in present disclosure can block the TSLP binding to cell surface of 293T-hTSLPR-hIL7R.

### 7.6 Short Form TSLP Binding

Short form huTSLP (97-159) expressing cells were added to 96 well plate, and supernatant was discarded after centrifugation. 50 μl of the antibody solution diluted with D10 was transferred to cell plate, for incubation at 4° C. for 0.5 hours. The plate was washed with PBS after centrifugation for 3 times, and the supernatant was discarded after centrifugation. 50 ul secondary antibody was added to 96 well plate, for binding at 4° C. for 0.5 hours. The supernatant was removed after centrifugation, and the plate was washed with PBS for 3 times, and the supernatant was discarded after centrifugation. The cells were resuspended with 100 ul PBS and the fluorescence values were read by flow cytometry.

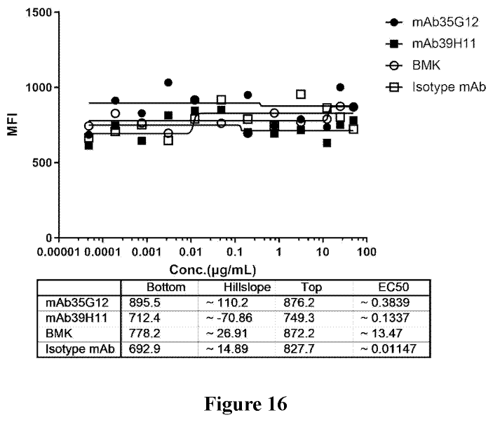

The results are shown in FIG. 16, which show that none of the antibodies mAb35G12 and mAb39H11 in present disclosure can bind to cell surface expressing short form huTSLP.

### 7.7 Digested TSLP Binding

To test antibody binding to digested form and non-digested form of TSLP, three different forms of TSLP were used in this study. H52Ha has an amino acid sequence of SEQ ID NO: 73, and is a mutated long-form TSLP with R127A and R130A mutations which confer resistance to enzymatic digestion. H52Hb has an amino acid sequence of SEQ ID NO: 82, and is a native long-form TSLP which contains a cleavage site at 130 and is prone to enzymatic digestion.

The TSLP to be digested H53Hb (ACRO #tsp-h52hb) comprises an amino acid sequence as shown in SEQ ID NO: 82. The non-digested TSLP H53Ha (ACRO #tsp-h52ha) comprises an amino acid sequence as shown in SEQ ID NO: 73.

(SEQ ID NO: 82)
YDFTNCDFEKIKAAYLSTISKDLITYMSGTKSTEFNNTVSCSNRPHCL
TEIQSLTFNPTAGCASLAKEMFAMKTKAALAIWCPGYSETQINATQAMK
KRRKRKVTTNKCLEQVSQLQGLWRRFNRPLLKQQ
(SEQ ID NO: 73)
YDFTNCDFEKIKAAYLSTISKDLITYMSGTKSTEFNNTVSCSNRPHCL
TEIQSLTFNPTAGCASLAKEMFAMKTKAALAIWCPGYSETQINATQAMK
KARKAKVTTNKCLEQVSQLQGLWRRFNRPLLKQQ

To obtain the digested product of native long-form TSLP H53Hb-d, a total system of 100 μL of digestion buffer (25 mm Tris, 1 mM CaCl2, 0.5% (w/V) Brij-35, pH 9.0) was prepared, in which the working concentration of long-form native TSLP (ACRO #tsp-h52hb) was 10 μg/ml and the working concentration of PCSK3 was 12 ug/ml, for enzymatic digestion at 37° C. for 24 hours. Then digested products (referred to as 52Hb-d) were prepared into western blot system (5 ug per well) of reducing and non-reducing samples respectively, and non-digested native TSLP H53Hb (ACRO #tsp-h52hb) and non-digested mutant TSLP H53Ha (ACRO #tsp-h52ha) were also added for SDS-PAGE electrophoresis in parallel.

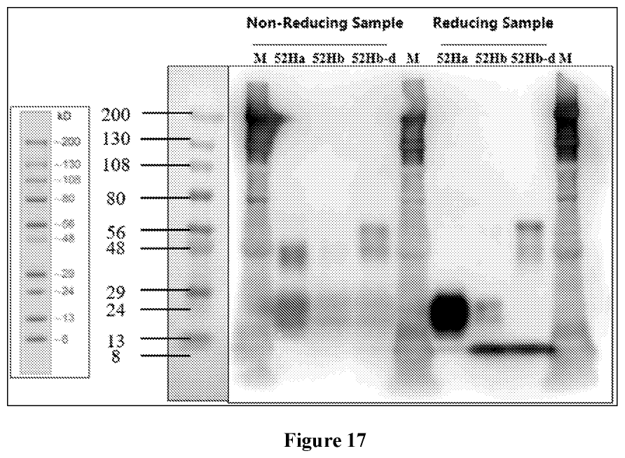

The enzyme digestion effect was detected by reduced SDS-PAGE. 52Ha showed no digested form fragment due to mutation, while wildtype sequence 52Hb showed significant digested fragments, which could attribute to the natural enzymes existing during production process. Moreover, 52Hb was further digested completely upon adding PCSK3, as shown in FIG. 17.

This result demonstrated that 52Hb comprises a mixture of non-digested and digested TSLP, while 52Hb-d is fully digested TSLP sample, both of which are further tested for anti-TSLP antibody binding activity.

Three forms of TSLP proteins (52Ha, 52Hb and 52Hb-d, respectively) were treated, loaded in SDS-PAGE protein loading buffer and prepared into reduced sample (containing DTT) and non-reduced sample (excluding DTT) with a concentration of 0.5 ug/20 ul. After SDS-PAGE electrophoresis, samples were transferred to membrane (PVDF membrane) using Beyotime membrane transfer device, set the membrane transfer current to 300 mA and the membrane transfer time to 60 min, and sealed (adding Western blocking solution, shaking slowly on the shaking table, blocking at 37° C. for more than 1 hour, and blocking at room temperature for more than 2 hours).

Primary antibody: BMK, 35G12Z03 and 39H11Z07 were diluted to the concentration of 2 ug/ml with antibody diluent respectively, incubated for 2h, washed with washing solution for 3 times (10 min each time). Each of the three antibodies here comprises an IgG2 heavy chain constant region (SEQ ID NO: 83) and an IgG2 light chain constant region (SEQ ID NO: 84).

(SEQ ID NO: 83)
ASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDKT
VERKCCVECPPCPAPPVAGPSVFLFPPKPKDTLMISRTPEVTCVVVDVS
HEDPEVQFNWYVDGVEVHNAKTKPREEQFNSTFRVVSVLTVVHQDWLNG
KEYKCKVSNKGLPAPIEKTISKTKGQPREPQVYTLPPSREEMTKNQVSL
TCLVKGFYPSDISVEWESNGQPENNYKTTPPMLDSDGSFFLYSKLTVDK
SRWQQGNVFSCSVMHEALHNHYTQKSLSLSPGK
(SEQ ID NO: 84)
RTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSS
PVTKSFNRGEC

Secondary antibody: the secondary antibody anti HuIgG HRP (Sino ssa002) 1:2000 were diluted with antibody diluent, incubated at room temperature for 60 min, washed with washing solution for 3 times (10 min each time).

Coloration using chemiluminescence method: the two anti PVDF membrane were placed in the fluorescence and chemiluminescence imaging system. luminescent liquid A and the B of the same volume were mixed and evenly distributed on the PVDF film. The software of the gel imaging system was run and the exposure time was set.

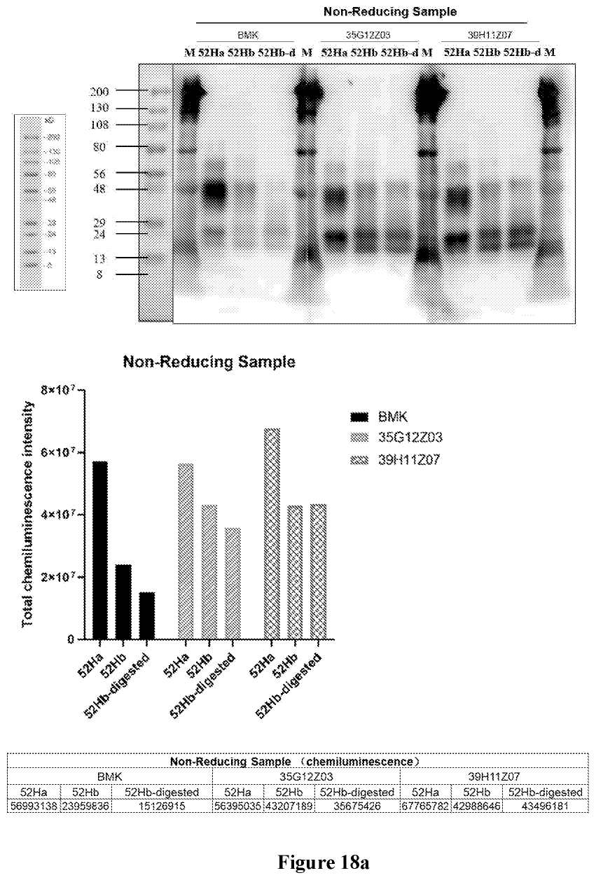

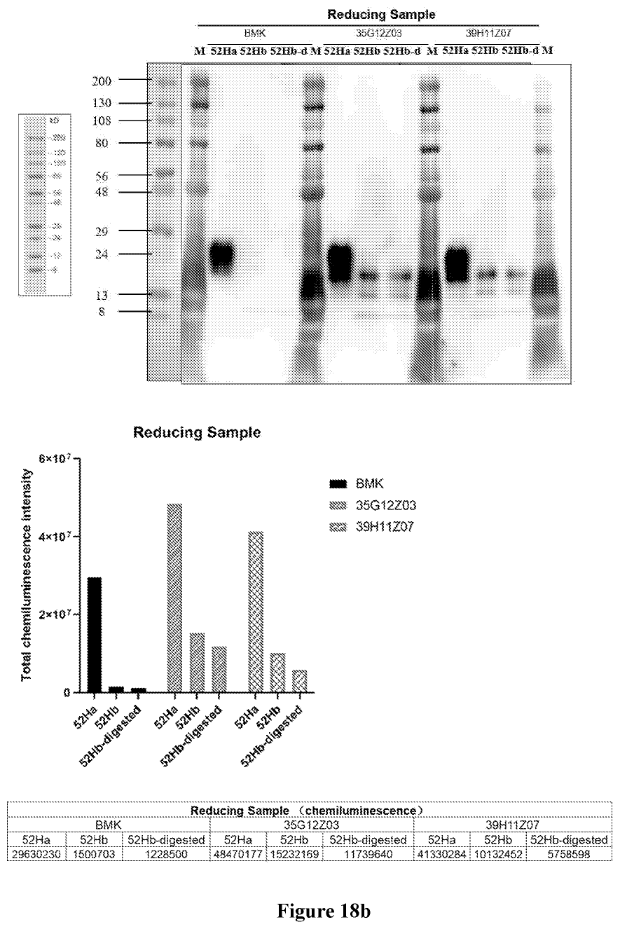

52Ha, 52Hb and 52Hb-d TSLP proteins were immunoblotted with primary and secondary antibodies as indicated above, under reducing and non-reducing conditions, respectively. The results are shown in FIGS. 18a (with non-reducing sample) and 18b (with reducing sample). Total chemiluminescence of each single lane was measured for gray value.

The results show that molecules in present disclosure could potently bind to the digested form of TSLP (52Hb-d). Such binding effect is much stronger (e.g. at least 2 fold) than that observed for the BMK molecule (see FIG. 18a, 18b).

### 7.8 STAT5 Activation Blocking

In order to determine the function of antibody blocking long-form TSLP stimulated STAT5 activation, 293T-hTSLPR-hIL7Ra and STAT5-Luciferase stable cells (constructed according to Section 2.4 of Example 2) in good growth condition were used. After trypsin digestion and cell counting, the cells were diluted to 2×10⁵/ml, and 15 ul/well cells were added to 384 well plate. The antibody solution was diluted with DMEM cell culture medium, and 15 ul of antibody solutions of different concentrations were added to 384 well plate, for incubation in incubator for 30 min. 30 ng/ml human TSLP (ACRO, tsp-h52ha) was prepared with DMEM cell culture medium, and 15 ul/well cells were added to 384 well plate and cultured at 37° C. with 5% CO₂ for 4-6h. Finally, 25 ul bright Glo™ Luciferase assay system (Promega, e2620) was added. After being shaked for 5 min, the luminescence values were read with enzyme labeling instrument.

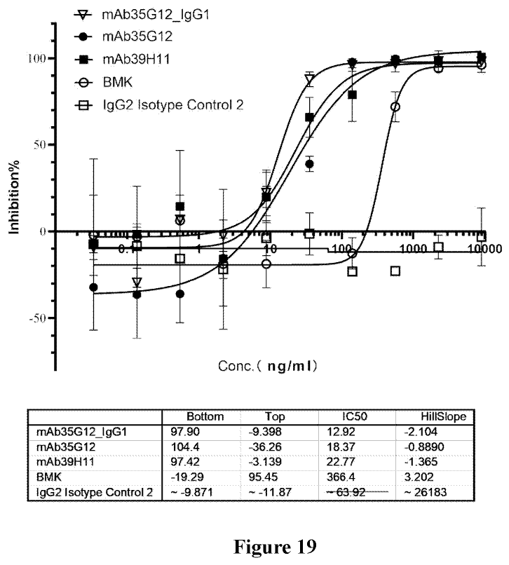

The experimental results are shown in the FIG. 19. According to the detection results, molecules mAb35G12 and mAb39H11 presented can well block the STAT5 activation stimulated by long-form TSLP. Switching the isotypes of mAb35G12 retained similar blocking potency. Compared with BMK antibody, the blocking IC50 of candidate clones mAb35G12, mAb35G12_IgG1 and mAb39H11 are significantly better.

### 7.9 Blockade of Natural TSLP-Induced TARC Secretion

Natural TSLP was induced and collected from human lung fibroblasts. Briefly, human lung fibroblasts with a density of 80% were taken out, digested with trypsin, and subcultured into T25 culture flask according to 1:3. The cell density was monitored and when it was up to 80%, the supernatant was discarded. Natural TSLP production was induced by culture human lung fibroblasts in medium containing 10 ng/ml IL1-β, 20 ng/ml IL-13, and 20 ng/ml TNF-α. After incubation for 24 hours, the TSLP induced supernatant was collected into the centrifuge tube, and then centrifuged at 3000 rpm for 10 min to transfer the supernatant to a new centrifuge tube.

The antibody was diluted with cell culture medium. PBMC, resuspend the cells with cell culture medium were thawed after centrifugation. The cell concentration was adjusted to 1×10⁷/ml, and added 50 ul/well to 96 well cell culture plate. Natural TSLP was prepared as described above, and was added 25 ul/well to 96 well cell culture plate to make the final concentration of 20 ng/ml. 25 ul diluted antibody solution was added per well. After 24 hours, TARC expression was detected by ELISA.

2 ug/ml capture antibody (Sino, sek10233) antigen solution was prepared with PBS, and coated in 384 well plate with 25 ul per well, for incubation overnight at 4° C. The supernatant was discarded. 80 ul/well 3% milk was added and sealed at room temperature for 1.5 hours. The supernatant was discarded and the plate was washed with PBST (0.1% T20) for 3 times. 25 ul of samples to be tested were added to 384 well plate for incubation for 2 hours at room temperature. The supernatant was discarded and the plate was washed with PBST (0.1% T20) for 3 times. 25 ul of secondary antibody solution was added to 384 well plate per well for incubation at room temperature for 1 hour. The supernatant was discarded and the plate was washed with PBST (0.1% T20) for 7 times. HRP substrate TMB was added 25 ul/well. After incubation for 12 minutes, 25 ul termination solution and 2m HCl were added to terminate the reaction. The microplate reader was used to read the absorbance value of 450 nm.

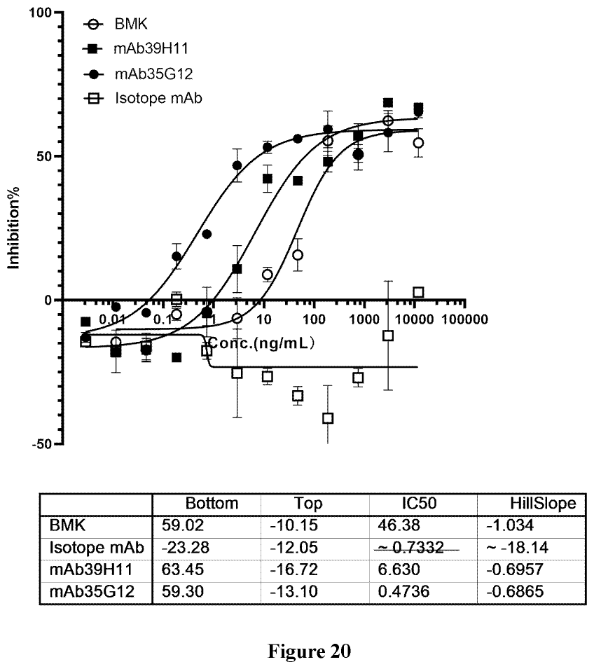

The results are shown in FIG. 20. The results showed that antibodies mAb35G12 and mAb39H11 in present disclosure can block natural TSLP induced TARC secretion, which is better than BMK.

### 7.10 Recombinant TSLP Dependent BaF3 Proliferation

The protocols of recombinant TSLP dependent BaF3 proliferation test for the humanized antibodies were the same as those described in Example 4.2.

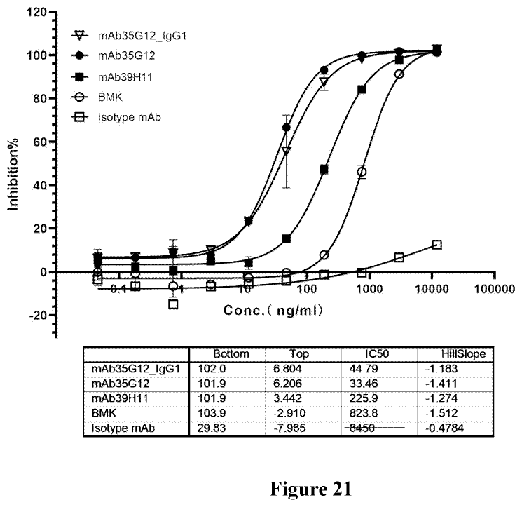

The results are shown in FIG. 21, which showed that antibodies mAb35G12, mAb35G12_IgG1 and mAb39H11 in present disclosure can block TSLP induced BaF3-hTSLP-hIL7R cell proliferation, which is better than BMK. Switching the isotypes of mAb35G12 retained relatively similar blocking potency.

### 7.11 Natural TSLP Dependent BaF3 Proliferation

Human lung fibroblasts with a density of 80% were taken out, digested with trypsin, and subcultured into T25 culture flask according to 1:3. The cell density was monitored and when it was up to 80%, the supernatant was discarded. TSLP production was induced by culture human lung fibroblasts in medium containing 10 ng/ml IL1-β, 20 ng/ml IL-13, and 20 ng/mL TNF-α. Another T25 bottle containing above media without cell was taken. Cells were cultured in a third T25 bottle containing serum-free medium. After incubation for 24 hours, the above cell supernatants (TSLP induced supernatant/non induced supernatant/cell-free supernatant) were collected into the centrifuge tube respectively, and then centrifuged at 3000 rpm for 10 min to transfer the supernatant to a new centrifuge tube. The antibody solutions were diluted with serum-free medium. Baf3-TSLPR-IL7Ra was thawed and washed twice with medium. The cells were resuspended and counted, and the cell density was adjusted to 4×10⁵/ml, added 30 μL/well into 384 well cell culture plate. TSLP induced supernatant was spread 15 ul/well in 384 well black cell culture plate (TSLP induced supernatant was added to the experimental well and TSLP induced supernatant, non-induced supernatant/cell-free supernatant was added to the control well respectively) and cultured in 37° C. and 5% CO₂ cell culture chamber for 2 days. The 384 well plate was taken out, added with 15 ul CTG, shaken for 5 min, and the luminous values were red within 30 min. The proliferation of baf3 was detected.

The results showed that only fibroblasts induced by cytokines could stimulate the proliferation of Baf3, indicating that this method could induce the expression of natural TSLP.

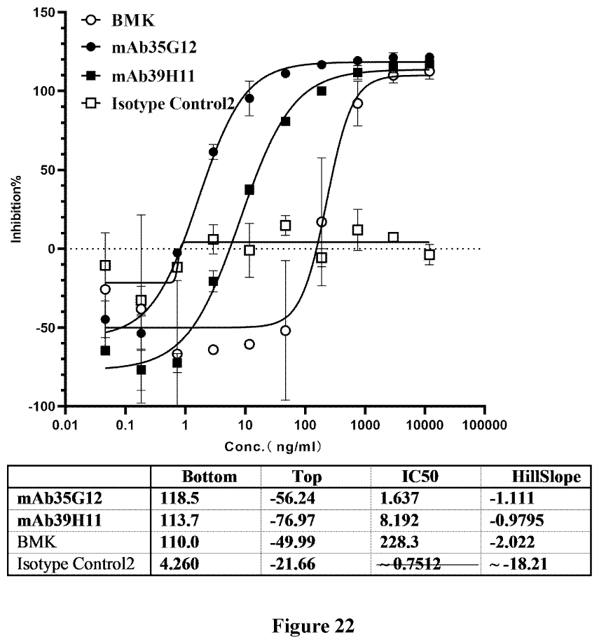

Then we tested the inhibitory effect of antibody on Baf3 proliferation induced by natural TSLP by the same method as that of antibody inhibiting hTSLP protein stimulating baf3 proliferation experiment (see Example 4.3). The results are shown in FIG. 22, which show that molecules mAb35G12 and mAb39H11 in present disclosure could potently block the proliferation of baf3 cells. Molecules presented were better than BMK in inhibitory effect.

What is claimed is:

1. An anti-TSLP antibody or antigen-binding fragment thereof, comprising heavy chain complementary determining region 1 (HCDR1), HCDR2 and HCDR3, wherein
(a) the HCDR1 comprises an amino acid sequence of SEQ ID NO: 11;
the HCDR2 comprises an amino acid sequence of IFPGDGX₁T (SEQ ID NO: 59); and
the HCDR3 comprises an amino acid sequence of ARX₂GX₃X₄X₅X₆X₇X₈YAMDY (SEQ ID NO: 60);
wherein, X₁ is E or D; X₂ is G or S; X₃ is Y or F; X₄ is V or D; X₅ is N or Y; X₆ is none or G; X₇ is F or Y; X₈ is L or F; or
(b) the HCDR1 comprises an amino acid sequence of SYWX₉N (SEQ ID NO: 61);
the HCDR2 comprises an amino acid sequence of QIFPGDGX₁₀TX₁₁Y NGX₁₂FKG (SEQ ID NO: 62); and
the HCDR3 comprises an amino acid sequence of X₁₃GX₁₄X₁₅X₁₆X₁₇X₁₈X₁₉YAMDY (SEQ ID NO: 63);
wherein, X₉ is M or I; X₁₀ is E or D; X₁₁ is N or T; X₁₂ is K or N; X₁₃ is G or S; X₁₄ is Y or F; X₁₅ is V or D; X₁₆ is N or Y; X₁₇ is none or G; X₁₈ is F or Y; X₁₉ is L or F.

2. An anti-TSLP antibody or antigen-binding fragment thereof, comprising light chain complementary determining region 1 (LCDR1), LCDR2 and LCDR3, wherein
(a) the LCDR1 comprises an amino acid sequence of SEQ ID NO: 14;
the LCDR2 comprises an amino acid sequence of SEQ ID NO: 15; and
the LCDR3 comprises an amino acid sequence of SEQ ID NO: 16; or
(b) the LCDR1 comprises an amino acid sequence of SEQ ID NO: 53;
the LCDR2 comprises an amino acid sequence of GTSX₂₀LAS (SEQ ID NO: 64); and
the LCDR3 comprises an amino acid sequence of SEQ ID NO: 16;
wherein, X₂₀ is T or N.

3. The antibody or antigen-binding fragment thereof of claim 1 or 2, wherein
(a) the HCDR1 comprises an amino acid sequence of SEQ ID NO: 11;
the HCDR2 comprises an amino acid sequence of IFPGDGX₁T (SEQ ID NO: 59); and
the HCDR3 comprises an amino acid sequence of ARX₂GX₃X₄X₅X₆X₇X₈YAMDY (SEQ ID NO: 60);
the LCDR1 comprises an amino acid sequence of SEQ ID NO: 14,
the LCDR2 comprises an amino acid sequence of SEQ ID NO: 15, and
the LCDR3 comprises an amino acid sequence of SEQ ID NO: 16,
X₁ is E or D; X₂ is G or S; X₃ is Y or F; X₄ is V or D; X₅ is N or Y; X₆ is none or G; X₇ is F or Y; X₈ is L or F;
(b) the HCDR1 comprises an amino acid sequence of SYWX₉N (SEQ ID NO: 61);
the HCDR2 comprises an amino acid sequence of QIFPGDGX₁₀TX₁₁Y NGX₁₂FKG (SEQ ID NO: 62); and
the HCDR3 comprises an amino acid sequence of X₁₃GX₁₄X₁₅X₁₆X₁₇X₁₈X₁₉YAMDY (SEQ ID NO: 63);
the LCDR1 comprises an amino acid sequence of SEQ ID NO: 53;
the LCDR2 comprises an amino acid sequence of GTSX₂₀LAS (SEQ ID NO: 64); and
the LCDR3 comprises an amino acid sequence of SEQ ID NO: 16;
wherein, X₉ is M or I; X₁₀ is E or D; X₁₁ is N or T; X₁₂ is K or N; X₁₃ is G or S; X₁₄ is Y or F; X₁₅ is V or D; X₁₆ is N or Y; X₁₇ is none or G; X₁₈ is F or Y; X₁₉ is L or F; X₂₀ is T or N.

4. The antibody or antigen-binding fragment thereof of claim 3, wherein
(a) the HCDR1 comprises an amino acid sequence of SEQ ID NO: 11,
the HCDR2 comprises an amino acid sequence of SEQ ID NO: 12 or 25,
the HCDR3 comprises an amino acid sequence of SEQ ID NO: 13 or 26,
the LCDR1 comprises an amino acid sequence of SEQ ID NO: 14,
the LCDR2 comprises an amino acid sequence of SEQ ID NO: 15, and
the LCDR3 comprises an amino acid sequence of SEQ ID NO: 16; or
(b) the HCDR1 comprises an amino acid sequence of SEQ ID NO: 50 or 55,
the HCDR2 comprises an amino acid sequence of SEQ ID NO: 51 or 56,
the HCDR3 comprises an amino acid sequence of SEQ ID NO: 52 or 57,
the LCDR1 comprises an amino acid sequence of SEQ ID NO: 53,
the LCDR2 comprises an amino acid sequence of SEQ ID NO: 54 or 58, and
the LCDR3 comprises an amino acid sequence of SEQ ID NO: 16.

5. The antibody or antigen-binding fragment thereof of claim 3, wherein
(a) the HCDR1 comprises an amino acid sequence of SEQ ID NO: 11,
the HCDR2 comprises an amino acid sequence of SEQ ID NO: 12,
the HCDR3 comprises an amino acid sequence of SEQ ID NO: 13,
the LCDR1 comprises an amino acid sequence of SEQ ID NO: 14,
the LCDR2 comprises an amino acid sequence of SEQ ID NO: 15, and
the LCDR3 comprises an amino acid sequence of SEQ ID NO: 16; or
(b) the HCDR1 comprises an amino acid sequence of SEQ ID NO: 11,
the HCDR2 comprises an amino acid sequence of SEQ ID NO: 25,
the HCDR3 comprises an amino acid sequence of SEQ ID NO: 26,
the LCDR1 comprises an amino acid sequence of SEQ ID NO: 14,
the LCDR2 comprises an amino acid sequence of SEQ ID NO: 15, and
the LCDR3 comprises an amino acid sequence of SEQ ID NO: 16;
(c) the HCDR1 comprises an amino acid sequence of SEQ ID NO: 50,
the HCDR2 comprises an amino acid sequence of SEQ ID NO: 51,
the HCDR3 comprises an amino acid sequence of SEQ ID NO: 52,
the LCDR1 comprises an amino acid sequence of SEQ ID NO: 53,
the LCDR2 comprises an amino acid sequence of SEQ ID NO: 54, and
the LCDR3 comprises an amino acid sequence of SEQ ID NO: 16; or
(d) the HCDR1 comprises an amino acid sequence of SEQ ID NO: 55,
the HCDR2 comprises an amino acid sequence of SEQ ID NO: 56,
the HCDR3 comprises an amino acid sequence of SEQ ID NO: 57,
the LCDR1 comprises an amino acid sequence of SEQ ID NO: 53,
the LCDR2 comprises an amino acid sequence of SEQ ID NO: 58, and
the LCDR3 comprises an amino acid sequence of SEQ ID NO: 16.

6. The antibody or antigen-binding fragment thereof of claim 1, further comprising one or more of heavy chain framework region 1 (HFR1), HFR2, HFR3 and HFR4, and/or one or more of light chain framework region 1 (LFR1), LFR2, LFR3 and LFR4, wherein
the HFR1 comprises an amino acid sequence of SEQ ID NO: 17, SEQ ID NO: 27, or X₂₁VQLVQSGAEVKKPGX₂₂SX₂₃KX₂₄SCKX₂₅S (SEQ ID NO: 65), or a homologous sequence of at least 85% sequence identity thereof,
the HFR2 comprises an amino acid sequence of SEQ ID NO: 18 or WVRQX₂₇PGX₂₈GLEWMG (SEQ ID NO: 66), or a homologous sequence of at least 85% sequence identity thereof,
the HFR3 comprises an amino acid sequence of SEQ ID NO: 19, SEQ ID NO: 28, or X₃₁VTX₃₂X₃₃X₃₄DX₃₅SX₃₆STX₃₇YX₃₈X₃₉X₄₀SSLX₄₁X₄₂X₄₃DTAX₄₄YYC (SEQ ID NO: 67), or a homologous sequence of at least 85% sequence identity thereof,
the HFR4 comprises an amino acid sequence of SEQ ID NO: 20 or SEQ ID NO: 35, or a homologous sequence of at least 85% sequence identity thereof,
the LFR1 comprises an amino acid sequence of SEQ ID NO: 21, SEQ ID NO: 29, or SEQ ID NO: 36, or a homologous sequence of at least 85% sequence identity thereof,
the LFR2 comprises an amino acid sequence of SEQ ID NO: 22, SEQ ID NO: 30, or WYQQKPGQSPRPWIX₄₅ (SEQ ID NO: 68), or a homologous sequence of at least 85% sequence identity thereof,
the LFR3 comprises an amino acid sequence of SEQ ID NO: 23, SEQ ID NO: 31, or SEQ ID NO:38, or a homologous sequence of at least 85% sequence identity thereof, and
the LFR4 comprises an amino acid sequence of SEQ ID NO: 24 or SEQ ID NO: 39, or a homologous sequence of at least 85% sequence identity thereof,
wherein, X₂₁ is E or Q; X₂₂ is E, S or A; X₂₃ is L or V; X₂₄ is I or V; X₂₅ is S or A; X₂₇ is M or A; X₂₈ is K or Q; X₃₁ is Q or R; X₃₂ is I or M; X₃₃ is S or T; X₃₄ is A or R; X₃₅ is K or T; X₃₆ is I or T; X₃₇ is A or V; X₃₈ is L or M; X₃₉ is Q or E; X₄₀ is W or L; X₄₁ is K or R; X₄₂ is A or S; X₄₃ is S or E; X₄₄ is M or V; X₄₅ is Y or F.

7. The antibody or antigen-binding fragment thereof of claim 6, wherein
the HFR1 comprises an amino acid sequence of SEQ ID NO: 17, 27, 32, 40, 42, 47 or 87,
the HFR2 comprises an amino acid sequence of SEQ ID NO: 18, 33, 43 or 48,
the HFR3 comprises an amino acid sequence of SEQ ID NO: 19, 28, 34, 41, 44 or 49,
the HFR4 comprises an amino acid sequence of SEQ ID NO: 20 or 35,
the LFR1 comprises an amino acid sequence of SEQ ID NO: 21, 29 or 36,
the LFR2 comprises an amino acid sequence of SEQ ID NO: 22, 30, 37 or 45,
the LFR3 comprises an amino acid sequence of SEQ ID NO: 23, 31, or 38, and
the LFR4 comprises an amino acid sequence of SEQ ID NO: 24 or 39.

8. The antibody or antigen-binding fragment thereof of claim 6 or 7, wherein
(a) the HFR1 comprises an amino acid sequence of SEQ ID NO: 17,
the HFR2 comprises an amino acid sequence of SEQ ID NO: 18,
the HFR3 comprises an amino acid sequence of SEQ ID NO: 19,
the HFR4 comprises an amino acid sequence of SEQ ID NO: 20,
the LFR1 comprises an amino acid sequence of SEQ ID NO: 21,
the LFR2 comprises an amino acid sequence of SEQ ID NO: 22,
the LFR3 comprises an amino acid sequence of SEQ ID NO: 23, and
the LFR4 comprises an amino acid sequence of SEQ ID NO: 24; or
(b) the HFR1 comprises an amino acid sequence of SEQ ID NO: 27,
the HFR2 comprises an amino acid sequence of SEQ ID NO: 18,
the HFR3 comprises an amino acid sequence of SEQ ID NO: 28,
the HFR4 comprises an amino acid sequence of SEQ ID NO: 20,
the LFR1 comprises an amino acid sequence of SEQ ID NO: 29,
the LFR2 comprises an amino acid sequence of SEQ ID NO: 30,
the LFR3 comprises an amino acid sequence of SEQ ID NO: 31, and
the LFR4 comprises an amino acid sequence of SEQ ID NO: 24;
(c) the HFR1 comprises an amino acid sequence of SEQ ID NO: 32,
the HFR2 comprises an amino acid sequence of SEQ ID NO: 33,
the HFR3 comprises an amino acid sequence of SEQ ID NO: 34,
the HFR4 comprises an amino acid sequence of SEQ ID NO: 35,
the LFR1 comprises an amino acid sequence of SEQ ID NO: 36,
the LFR2 comprises an amino acid sequence of SEQ ID NO: 37,
the LFR3 comprises an amino acid sequence of SEQ ID NO: 38, and
the LFR4 comprises an amino acid sequence of SEQ ID NO: 39;
(d) the HFR1 comprises an amino acid sequence of SEQ ID NO: 40,
the HFR2 comprises an amino acid sequence of SEQ ID NO: 33,
the HFR3 comprises an amino acid sequence of SEQ ID NO: 41,
the HFR4 comprises an amino acid sequence of SEQ ID NO: 35,
the LFR1 comprises an amino acid sequence of SEQ ID NO: 36,
the LFR2 comprises an amino acid sequence of SEQ ID NO: 37,
the LFR3 comprises an amino acid sequence of SEQ ID NO: 38, and
the LFR4 comprises an amino acid sequence of SEQ ID NO: 39;
(e) the HFR1 comprises an amino acid sequence of SEQ ID NO: 42,
the HFR2 comprises an amino acid sequence of SEQ ID NO: 43,
the HFR3 comprises an amino acid sequence of SEQ ID NO: 44,
the HFR4 comprises an amino acid sequence of SEQ ID NO: 35,
the LFR1 comprises an amino acid sequence of SEQ ID NO: 36,
the LFR2 comprises an amino acid sequence of SEQ ID NO: 45,
the LFR3 comprises an amino acid sequence of SEQ ID NO: 38, and
the LFR4 comprises an amino acid sequence of SEQ ID NO: 39;
(f) the HFR1 comprises an amino acid sequence of SEQ ID NO: 87,
the HFR2 comprises an amino acid sequence of SEQ ID NO: 33,
the HFR3 comprises an amino acid sequence of SEQ ID NO: 41,
the HFR4 comprises an amino acid sequence of SEQ ID NO: 35,
the LFR1 comprises an amino acid sequence of SEQ ID NO: 36,
the LFR2 comprises an amino acid sequence of SEQ ID NO: 45,
the LFR3 comprises an amino acid sequence of SEQ ID NO: 38, and
the LFR4 comprises an amino acid sequence of SEQ ID NO: 39; or
(g) the HFR1 comprises an amino acid sequence of SEQ ID NO: 47,
the HFR2 comprises an amino acid sequence of SEQ ID NO: 48,
the HFR3 comprises an amino acid sequence of SEQ ID NO: 49,
the HFR4 comprises an amino acid sequence of SEQ ID NO: 35,
the LFR1 comprises an amino acid sequence of SEQ ID NO: 36,
the LFR2 comprises an amino acid sequence of SEQ ID NO: 45,
the LFR3 comprises an amino acid sequence of SEQ ID NO: 38, and
the LFR4 comprises an amino acid sequence of SEQ ID NO: 39.

9. The antibody or antigen-binding fragment thereof of any of the preceding claims, comprising a heavy chain variable region (VH) comprising an amino acid sequence selected from the group consisting of SEQ ID NOs: 1, 3, 5, 7, 8, 10 and 86, or a homologous sequence thereof having at least 80% sequence identity.

10. The antibody or antigen-binding fragment thereof of any of the preceding claims, comprising a light chain variable region (VL) comprising an amino acid sequence selected from the group consisting of SEQ ID NOs: 2, 4, 6 and 9, or a homologous sequence thereof having at least 80% sequence identity.

11. The antibody or an antigen-binding fragment thereof of any of the preceding claims, wherein at least one of the substitutions or modifications is in one or more of the complementary determining region (CDR) sequences of the heavy chain variable region or light chain variable region.

12. The antibody or an antigen-binding fragment thereof of any of the preceding claims, wherein at least one of the substitutions or modifications is in one or more of the non-CDR sequences of the heavy chain variable region or light chain variable region.

13. The antibody or an antigen-binding fragment thereof of any one of the preceding claims, further comprising an Fc region, optionally an Fc region of human immunoglobulin (Ig), or optionally an Fc region of human IgG.

14. The antibody or an antigen-binding fragment thereof of claim 13, wherein the Fc region is derived from human IgG1 or IgG4.

15. The antibody or an antigen-binding fragment thereof of claim 14, wherein the Fc region is derived from human IgG4 and comprises one or more mutations selected from the group consisting of S228P, F234A, L235A, M252Y, S254T, T256E, K447del.

16. The antibody or an antigen-binding fragment thereof of claim 15, wherein the amino acid sequence of the Fc region derived from human IgG4 comprising mutations of S228P, F234A, L235A, M252Y, S254T, T256E, K447del is shown as SEQ ID NO: 75.

17. The antibody or antigen-binding fragment thereof of any of the preceding claims, further comprising a signal peptide at the N-terminal of the heavy chain variable region and/or a signal peptide at the N-terminal of the light chain variable region.

18. The antibody or an antigen-binding fragment thereof of any one of the preceding claims, which is humanized.

19. The antibody or an antigen-binding fragment thereof of any one of the preceding claims, which is a monoclonal antibody, a bispecific antibody, a multi-specific antibody, a recombinant antibody, a chimeric antibody, a labeled antibody, a bivalent antibody, an anti-idiotypic antibody or a fusion protein.

20. The antibody or an antigen-binding fragment thereof of any one of the preceding claims, which is a diabody, a Fab, a Fab′, a F(ab′)2, a Fd, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

21. The antibody or antigen-binding fragment thereof of any of the preceding claims, further comprising one or more amino acid residue substitutions or modifications yet retains binding specificity to human TSLP.

22. The antibody or an antigen-binding fragment thereof of any one of the preceding claims, having one or more properties selected from the group consisting of:
a) having the ability to specifically bind to human TSLP;
b) having the ability to block the binding between TSLP with TSLPR;
c) having the ability to block the binding between TSLP with cells expressing TSLPR and IL7R;
d) having the ability to inhibit the TSLP dependent proliferation of BaF3 cells;
e) having the ability to inhibit the TSLP dependent TARC secretion from PBMC;
or
f) having the ability to inhibit the TSLP dependent STAT5 activation in cells expressing TSLPR and IL7R.

23. The antibody or antigen-binding fragment thereof of claim 18, wherein the antibody or antigen-binding fragment thereof does not specifically bind to TSLP of monkey, rat or mouse.

24. The antibody or antigen-binding fragment thereof of claim 18 or 19, wherein antibody or antigen-binding fragment thereof specifically binds to long-form human TSLP and does not bind to short-form human TSLP;
wherein, the long-form human TSLP has an amino acid sequence as shown in SEQ ID NO: 73, and the short-form human TSLP has an amino sequence as shown in SEQ ID NO: 74.

25. The antibody or antigen-binding fragment thereof of claim 20, having the ability to inhibit the human TSLP dependent proliferation of human TSLPR-expressing cells.

26. The antibody or antigen-binding fragment thereof of claim 23, wherein the cells further express human IL7R.

27. The antibody or an antigen-binding fragment thereof of any one of the preceding claims, binding to a different epitope from what a reference antibody binds, wherein the amino acid sequence of the heavy chain of reference antibody is shown as SEQ ID NO: 77, and the amino acid sequence of the light chain of reference antibody is shown as SEQ ID NO: 78.

28. The antibody or an antigen-binding fragment thereof of any one of the preceding claims, which is linked to one or more conjugate moieties.

29. The antibody or an antigen-binding fragment thereof of claim 26, wherein the conjugate moiety comprises an agent for detection or isolation, such as a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, or other anticancer drugs.

30. An isolated polynucleotide encoding the antibody or an antigen-binding fragment thereof of any one of claims 1-29.

31. A vector comprising the isolated polynucleotide of claim 30.

32. A host cell comprising the vector of claim 31.

33. A pharmaceutical composition, comprising:
(i) the antibody or an antigen-binding fragment thereof of any one of claims 1-29, or the polynucleotide encoding the antibody or antigen-binding fragment thereof of any one of claims 1-29; and
(ii) one or more pharmaceutically acceptable carriers, diluent, buffer or excipient.

34. The pharmaceutical composition of claim 33, further comprising an additional therapeutic agent.

35. The pharmaceutical composition of claim 34, wherein the additional therapeutic agent is an agent for treating an inflammatory disease, an autoimmune disease, and a cancer.

36. The pharmaceutical composition of claim 35, wherein the additional therapeutic agent is an agent targeting IL-33, IL-25, IL-4, IL-5, IL-4R, or IL-13.

37. A method of expressing the antibody or an antigen-binding fragment thereof of any one of claims 1-29, comprising culturing the host cell of claim 34 under the condition at which the vector of claim 31 is expressed.

38. A method of treating, preventing or alleviating a TSLP-related disease or disorder in a subject, comprising administering to the subject a therapeutically effective amount of the antibody or an antigen-binding fragment thereof capable of binding to long-form human TSLP having an amino acid sequence as shown in SEQ ID NO: 73, and a digested form of TSLP.

39. A method of treating, preventing or alleviating a TSLP-related disease or disorder in a subject, comprising administering to the subject a therapeutically effective amount of the antibody or an antigen-binding fragment thereof of any one of claims 1-29, or the polynucleotide encoding the antibody or antigen-binding fragment thereof of any one of claims 1-29, and/or the pharmaceutical composition of any one of claim 33-36.

40. The method of any one of claims 37-39, wherein the disease or disorder is associated with dysregulation of TSLPR mediated signaling compared to the control level.

41. The method of claim 40, wherein the dysregulation of TSLP mediated signaling includes dysregulation of STAT5 activation by phosphorylation.

42. The method of claim 40, wherein the dysregulation of TSLP mediated signaling includes dysregulation of TSLPR-expressing cell proliferation.

43. The method of any one of claims 37-39, wherein the disease or disorder is selected from the group consisting of: an inflammatory disease, an autoimmune disease, and a cancer.

44. The method of claim 43, wherein the disease or disorder is selected from the group consisting of: asthma (including severe asthma), idiopathic pulmonary fibrosis, atopic dermatitis (AD), allergic conjunctivitis, allergic rhinitis (AR), Netherton syndrome (NS), eosinophilic esophagitis (EoE), food allergy, allergic diarrhoea, eosinophilic gastroenteritis, allergic bronchopulmonary aspergillosis (ABPA), allergic fungal sinusitis, chronic pruritus, cancer, rheumatoid arthritis, Chronic obstructive pulmonary disease COPD, systemic sclerosis, multiple sclerosis, keloids, ulcerative colitis, chronic rhinosinusitis (CRS), polyposis, chronic eosinophilic pneumonia, eosinophilic bronchitis, allergic bronchopulmonary aspergillosis, coeliac disease, eosinophilic gastroenteritis, ChurgStrauss syndrome, eosinophilic myalgia syndrome, hypereosinophilic syndrome, eosinophilic granulomatosis with polyangiitis, eosinophilic esophagitis, inflammatory bowel disease, fibrotic disorder, inflammatory bowel disease Hodgkin's lymphoma, systemic lupus erythematosus.

45. The method of claim 44, wherein the cancer is selected from: breast cancer, pancreas cancer, colon cancer, lung cancer, ovarian cancer, prostate cancer, and B-cell acute lymphoblastic leukemia.

46. The method of claim 44, wherein the fibrotic disorder is selected from: systemic and local scleroderma, keloids and hypertrophic scars, interstitial lung disease (ILD), idiopathic pulmonary fibrosis (IPF), liver fibrosis resulting from chronic hepatitis B or C infection, radiation-induced fibrosis, and fibrosis arising from wound healing, atherosclerosis, restinosis, pulmonary inflammation and fibrosis, liver cirrhosis, kidney disease, heart disease resulting from scar tissue, and eye diseases such as macular degeneration, and retinal and vitreal retinopathy, fibrosis resulting from chemotherapeutic drugs, and injuries and burns.

47. The method of claim 39, wherein the disease or disorder is selected from the group consisting of asthma, Polyp nasal Sinusitis, COPD, urticaria, EoE, and atopic dermatitis.

48. The method of any one of claims 37-47, wherein the subject is human.

49. The method of any one of claims 37-48, wherein the administration is via oral, nasal, intravenous, subcutaneous, sublingual, or intramuscular administration.

50. A method of detecting the presence or amount of TSLP in a sample, comprising contacting the sample with the antibody or an antigen-binding fragment thereof of any one of claims 1-29, and determining the presence or the amount of TSLP in the sample.

51. The method of claim 50, further comprising a step of determining whether TSLP is over-expressed in the cells in the sample.

52. Use of the antibody or an antigen-binding fragment thereof of any one of claims 1-29, the pharmaceutical composition of any one of claims 33-36, and/or the polynucleotide encoding the antibody or antigen-binding fragment thereof of any one of claims 1-29 in the manufacture of a medicament for treating, preventing or alleviating a disease or disorder that is responsive to TSLP inhibition.

53. A chimeric antigen receptor (CAR) comprising an antigen binding domain, a transmembrane domain, and a TCR signaling domain, wherein the antigen binding domain specifically binds to TSLP and comprises an antigen binding fragment of any of claims 1-29.

54. The CAR of claim 53, wherein the CAR further comprises a costimulatory domain.

55. A nucleic acid sequence encoding the chimeric antigen receptor (CAR) of claim 53 or 54.

56. A cell comprising the nucleic acid sequence of claim 55.

57. The cell genetically modified to express the CAR of claim 53 or 54.

58. A vector comprising the nucleic acid sequence of claim 55.

59. A method for stimulating a T cell-mediated immune response to a TSLP-rich environment or tissue in a mammal, the method comprising administering to the mammal an effective amount of a cell genetically modified to express the CAR of claim 53 or 54.

60. A method of treating a mammal having a disease or disorder that is responsive to TSLP inhibition, comprising administering to the mammal an effective amount of a cell of claim 56, thereby treating the mammal.

61. The method of claim 60, wherein the cell is an autologous T cell.

62. The method of claim 60, wherein the mammal is a human subject.

63. The method of claim 60, wherein the mammal is identified as having a TSLP positive cell, or a cell with TSLP signaling up-regulated.


SEQUENCE LISTING (88 sequences)

SEQ ID NO: 1 - protein, 121 aa, synthetic construct
  REGION 1..121: note = synthetic
QVQLQQSGAELVRPGSSVKISCKASGYAFSSYWMNWVKQRPGQGLEWIGQIFPGDGETNYNGKFKGKATLTADKSSSTAYMQLSSLTSEDSAVYFCARGGYVNFLYAMDYWGQGTSVTVSS

SEQ ID NO: 2 - protein, 108 aa, synthetic construct
  REGION 1..108: note = synthetic
EIVLTQSPALMAASPGEKVTITCSVSSSISSSNLHWYQQKSETSPKPWIYGTSTLASGVPVRFSGSGSGTSYSLTISSMEAEDAATYYCQQWSSYPLTFGAGTKLELK

SEQ ID NO: 3 - protein, 122 aa, synthetic construct
  REGION 1..122: note = synthetic
QVQLQQSGAELVRPGSSVKISCKSSGYAFSSYWMNWVKQRPGQGLEWIGQIFPGDGDTTYNGNFKGKTTLTADKSSNTAYMQLSSLTSEDSAVYFCARSGFDYGYFYAMDYWGQGTSVTVSS

SEQ ID NO: 4 - protein, 108 aa, synthetic construct
  REGION 1..108: note = synthetic
EIVLTQSPTLMAASPGEKVTITCSVSSSISSSNLHWYQQKSETSPKPWIFGTSNLASGVPVRFSGSGSGTSYSLTISNMEAEDAATYYCQQWSSYPLTFGAGTKLELK

SEQ ID NO: 5 - protein, 121 aa, synthetic construct
  REGION 1..121: note = synthetic
QVQLVQSGAEVKKPGSSVKVSCKASGYAFSSYWMNWVRQAPGQGLEWMGQIFPGDGETNYNGKFKGRVTITADKSTSTAYMELSSLRSEDTAVYYCARGGYVNFLYAMDYWGQGTTVTVSS

SEQ ID NO: 6 - protein, 108 aa, synthetic construct
  REGION 1..108: note = synthetic
EIVMTQSPATLSVSPGERATLSCSVSSSISSSNLHWYQQKPGQSPRPWIYGTSTLASGIPARFSGSGSGTEFTLTISSLQSEDFAVYYCQQWSSYPLTFGGGTKVEIK

SEQ ID NO: 7 - protein, 121 aa, synthetic construct
  REGION 1..121: note = synthetic
QVQLVQSGAEVKKPGASVKVSCKASGYAFSSYWMNWVRQAPGQGLEWMGQIFPGDGETNYNGKFKGRVTMTRDTSTSTVYMELSSLRSEDTAVYYCARGGYVNFLYAMDYWGQGTTVTVSS

SEQ ID NO: 8 - protein, 122 aa, synthetic construct
  REGION 1..122: note = synthetic
EVQLVQSGAEVKKPGESLKISCKSSGYAFSSYWINWVRQMPGKGLEWMGQIFPGDGDTTYNGNFKGQVTISADKSISTAYLQWSSLKASDTAMYYCARSGFDYGYFYAMDYWGQGTTVTVSS

SEQ ID NO: 9 - protein, 108 aa, synthetic construct
  REGION 1..108: note = synthetic
EIVMTQSPATLSVSPGERATLSCSVSSSISSSNLHWYQQKPGQSPRPWIFGTSNLASGIPARFSGSGSGTEFTLTISSLQSEDFAVYYCQQWSSYPLTFGGGTKVEIK

SEQ ID NO: 10 - protein, 122 aa, synthetic construct
  REGION 1..122: note = synthetic
QVQLVQSGAEVKKPGSSVKVSCKSSGYAFSSYWINWVRQAPGQGLEWMGQIFPGDGDTTYNGNFKGRVTITADKSTSTAYMELSSLRSEDTAVYYCARSGFDYGYFYAMDYWGQGTTVTVSS

SEQ ID NO: 11 - protein, 8 aa, synthetic construct
  REGION 1..8: note = synthetic
GYAFSSYW

SEQ ID NO: 12 - protein, 8 aa, synthetic construct
  REGION 1..8: note = synthetic
IFPGDGET

SEQ ID NO: 13 - protein, 14 aa, synthetic construct
  REGION 1..14: note = synthetic
ARGGYVNFLYAMDY

SEQ ID NO: 14 - protein, 7 aa, synthetic construct
  REGION 1..7: note = synthetic
SSISSSN

SEQ ID NO: 15 - skipped

SEQ ID NO: 16 - protein, 9 aa, synthetic construct
  REGION 1..9: note = synthetic
QQWSSYPLT

SEQ ID NO: 17 - protein, 25 aa, synthetic construct
  REGION 1..25: note = synthetic
QVQLQQSGAELVRPGSSVKISCKAS

SEQ ID NO: 18 - protein, 17 aa, synthetic construct
  REGION 1..17: note = synthetic
MNWVKQRPGQGLEWIGQ

SEQ ID NO: 19 - protein, 38 aa, synthetic construct
  REGION 1..38: note = synthetic
NYNGKFKGKATLTADKSSSTAYMQLSSLTSEDSAVYFC

SEQ ID NO: 20 - protein, 11 aa, synthetic construct
  REGION 1..11: note = synthetic
WGQGTSVTVSS

SEQ ID NO: 21 - protein, 26 aa, synthetic construct
  REGION 1..26: note = synthetic
EIVLTQSPALMAASPGEKVTITCSVS

SEQ ID NO: 22 - protein, 17 aa, synthetic construct
  REGION 1..17: note = synthetic
LHWYQQKSETSPKPWIY

SEQ ID NO: 23 - protein, 36 aa, synthetic construct
  REGION 1..36: note = synthetic
TLASGVPVRFSGSGSGTSYSLTISSMEAEDAATYYC

SEQ ID NO: 24 - protein, 10 aa, synthetic construct
  REGION 1..10: note = synthetic
FGAGTKLELK

SEQ ID NO: 25 - protein, 8 aa, synthetic construct
  REGION 1..8: note = synthetic
IFPGDGDT

SEQ ID NO: 26 - protein, 15 aa, synthetic construct
  REGION 1..15: note = synthetic
ARSGFDYGYFYAMDY

SEQ ID NO: 27 - protein, 25 aa, synthetic construct
  REGION 1..25: note = synthetic
QVQLQQSGAELVRPGSSVKISCKSS

SEQ ID NO: 28 - protein, 38 aa, synthetic construct
  REGION 1..38: note = synthetic
TYNGNFKGKTTLTADKSSNTAYMQLSSLTSEDSAVYFC

SEQ ID NO: 29 - protein, 26 aa, synthetic construct
  REGION 1..26: note = synthetic
EIVLTQSPTLMAASPGEKVTITCSVS

SEQ ID NO: 30 - protein, 17 aa, synthetic construct
  REGION 1..17: note = synthetic
LHWYQQKSETSPKPWIF

SEQ ID NO: 31 - protein, 36 aa, synthetic construct
  REGION 1..36: note = synthetic
NLASGVPVRFSGSGSGTSYSLTISNMEAEDAATYYC

SEQ ID NO: 32 - protein, 25 aa, synthetic construct [sequence omitted]

SEQ ID NO: 33 - protein, 14 aa, synthetic construct
  REGION 1..14: note = synthetic
WVRQAPGQGLEWMG

SEQ ID NO: 34 - protein, 30 aa, synthetic construct
  REGION 1..30: note = synthetic
RVTITADKSTSTAYMELSSLRSEDTAVYYC

SEQ ID NO: 35 - protein, 11 aa, synthetic construct
  REGION 1..11: note = synthetic
WGQGTTVTVSS

SEQ ID NO: 36 - protein, 23 aa, synthetic construct
  REGION 1..23: note = synthetic
EIVMTQSPATLSVSPGERATLSC

SEQ ID NO: 37 - protein, 15 aa, synthetic construct
  REGION 1..15: note = synthetic
WYQQKPGQSPRPWIY

SEQ ID NO: 38 - protein, 32 aa, synthetic construct
  REGION 1..32: note = synthetic
GIPARFSGSGSGTEFTLTISSLQSEDFAVYYC

SEQ ID NO: 39 - protein, 10 aa, synthetic construct
  REGION 1..10: note = synthetic
FGGGTKVEIK

SEQ ID NO: 40 - protein, 25 aa, synthetic construct [sequence omitted]

SEQ ID NO: 41 - protein, 30 aa, synthetic construct
  REGION 1..30: note = synthetic
RVTMTRDTSTSTVYMELSSLRSEDTAVYYC

SEQ ID NO: 42 - protein, 25 aa, synthetic construct
  REGION 1..25: note = synthetic
EVQLVQSGAEVKKPGESLKISCKSS

SEQ ID NO: 43 - protein, 14 aa, synthetic construct
  REGION 1..14: note = synthetic
WVRQMPGKGLEWMG

SEQ ID NO: 44 - protein, 30 aa, synthetic construct
  REGION 1..30: note = synthetic
QVTISADKSISTAYLQWSSLKASDTAMYYC

SEQ ID NO: 45 - protein, 15 aa, synthetic construct
  REGION 1..15: note = synthetic
WYQQKPGQSPRPWIF

SEQ ID NO: 46 - skipped

SEQ ID NO: 47 - protein, 25 aa, synthetic construct
  REGION 1..25: note = synthetic
QVQLVQSGAEVKKPGSSVKVSCKSS

SEQ ID NO: 48 - protein, 14 aa, synthetic construct
  REGION 1..14: note = synthetic
WVRQAPGQGLEWMG

SEQ ID NO: 49 - protein, 30 aa, synthetic construct; identical to SEQ ID NO: 34
  REGION 1..30: note = synthetic

SEQ ID NO: 50 - protein, 5 aa, synthetic construct
  REGION 1..5: note = synthetic
SYWMN

SEQ ID NO: 51 - protein, 17 aa, synthetic construct
  REGION 1..17: note = synthetic
QIFPGDGETNYNGKFKG

SEQ ID NO: 52 - protein, 12 aa, synthetic construct
  REGION 1..12: note = synthetic
GGYVNFLYAMDY

SEQ ID NO: 53 - protein, 12 aa, synthetic construct
  REGION 1..12: note = synthetic
SVSSSISSSNLH

SEQ ID NO: 54 - protein, 7 aa, synthetic construct
  REGION 1..7: note = synthetic
GTSTLAS

SEQ ID NO: 55 - protein, 5 aa, synthetic construct
  REGION 1..5: note = synthetic
SYWIN

SEQ ID NO: 56 - protein, 17 aa, synthetic construct
  REGION 1..17: note = synthetic
QIFPGDGDTTYNGNFKG

SEQ ID NO: 57 - protein, 13 aa, synthetic construct
  REGION 1..13: note = synthetic
SGFDYGYFYAMDY

SEQ ID NO: 58 - protein, 7 aa, synthetic construct
  REGION 1..7: note = synthetic
GTSNLAS

SEQ ID NO: 59 - protein, 8 aa, synthetic construct
  REGION 1..8: note = synthetic
  SITE 7: note = misc_feature - Xaa can be Glu or Asp
IFPGDGXT

SEQ ID NO: 60 - protein, 15 aa, synthetic construct
  REGION 1..15: note = synthetic
  SITE 3: note = misc_feature - Xaa can be Gly or Ser
  SITE 5: note = misc_feature - Xaa can be Tyr or Phe
  SITE 6: note = misc_feature - Xaa can be Val or Asp
  SITE 7: note = misc_feature - Xaa can be Asn or Tyr
  SITE 8: note = misc_feature - Xaa can be none or Gly
  SITE 9: note = misc_feature - Xaa can be Phe or Tye
  SITE 10: note = misc_feature - Xaa can be Leu or Phe
ARXGXXXXXXYAMDY

SEQ ID NO: 61 - protein, 5 aa, synthetic construct
  REGION 1..5: note = synthetic
  SITE 4: note = misc_feature - Xaa can be Met or Ile
SYWXN

SEQ ID NO: 62 - protein, 17 aa, synthetic construct
  REGION 1..17: note = synthetic
  SITE 8: note = misc_feature - Xaa can be Glu or Asp
  SITE 10: note = misc_feature - Xaa can be Asn or Thr
  SITE 14: note = misc_feature - Xaa can be Lys or Asn
QIFPGDGXTXYNGXFKG

SEQ ID NO: 63 - protein, 13 aa, synthetic construct
  REGION 1..13: note = synthetic
  SITE 1: note = misc_feature - Xaa can be Gly or Ser
  SITE 3: note = misc_feature - Xaa can be Tyr or Phe
  SITE 4: note = misc_feature - Xaa can be Val or Asp
  SITE 5: note = misc_feature - Xaa can be Asn or Tyr
  SITE 6: note = misc_feature - Xaa can be none or Gly
  SITE 7: note = misc_feature - Xaa can be Phe or Tyr
  SITE 8: note = misc_feature - Xaa can be Leu or Phe
XGXXXXXXYAMDY

SEQ ID NO: 64 - protein, 7 aa, synthetic construct
  REGION 1..7: note = synthetic
  SITE 4: note = misc_feature - Xaa can be Thr or Asn
GTSXLAS

SEQ ID NO: 65 - protein, 25 aa, synthetic construct
  REGION 1..25: note = synthetic
  SITE 1: note = misc_feature - Xaa can be Glu or Gln
  SITE 16: note = misc_feature - Xaa can be Glu, Ser or Ala
  SITE 18: note = misc_feature - Xaa can be Leu or Val
  SITE 20: note = misc_feature - Xaa can be Ile or Val
  SITE 24: note = misc_feature - Xaa can be Ser or Ala
XVQLVQSGAEVKKPGXSXKXSCKXS

SEQ ID NO: 66 - protein, 14 aa, synthetic construct
  REGION 1..14: note = synthetic
  SITE 5: note = misc_feature - Xaa can be Met or Ala
  SITE 8: note = misc_feature - Xaa can be Lys or Gln
WVRQXPGXGLEWMG

SEQ ID NO: 67 - protein, 30 aa, synthetic construct
  REGION 1..30: note = synthetic
  SITE 1: note = misc_feature - Xaa can be Gln or Arg
  SITE 4: note = misc_feature - Xaa can be Ile or Met
  SITE 5: note = misc_feature - Xaa can be Ser or Thr
  SITE 6: note = misc_feature - Xaa can be Ala or Arg
  SITE 8: note = misc_feature - Xaa can be Lys or Thr
  SITE 10: note = misc_feature - Xaa can be Ile or Thr
  SITE 13: note = misc_feature - Xaa can be Ala or Val
  SITE 15: note = misc_feature - Xaa can be Leu or Met
  SITE 16: note = misc_feature - Xaa can be Gln or Glu
  SITE 17: note = misc_feature - Xaa can be Trp or Leu
  SITE 21: note = misc_feature - Xaa can be Lys or Arg
  SITE 22: note = misc_feature - Xaa can be Ala or Ser
  SITE 23: note = misc_feature - Xaa can be Ser or Glu
  SITE 27: note = misc_feature - Xaa can be Met or Val
XVTXXXDXSXSTXYXXXSSLXXXDTAXYYC

SEQ ID NO: 68 - protein, 17 aa, synthetic construct
  REGION 1..17: note = synthetic
  SITE 17: note = misc_feature - Xaa can be Tyr or Phe
LHWYQQKPGQSPRPWIX

SEQ ID NO: 69 - skipped

SEQ ID NO: 70 - protein, 507 aa, synthetic construct
  REGION 1..507: note = synthetic
MGWSCIILFLVATATGVHSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSK

SEQ ID NO: 71 - protein, 459 aa, synthetic construct
  REGION 1..459: note = synthetic
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 72 - protein, 371 aa, synthetic construct
  REGION 1..371: note = synthetic
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 73 - protein, 131 aa, synthetic construct
  REGION 1..131: note = synthetic
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKARKAKVTTNKCLEQVSQLQGLWRRFNRPLLKQQ

SEQ ID NO: 74 - protein, 63 aa, synthetic construct
  REGION 1..63: note = synthetic
MFAMKTKAALAIWCPGYSETQINATQAMKKARKAKVTTNKCLEQVSQLQGLWRRFNRPLLKQQ

SEQ ID NO: 75 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 76 - protein, 19 aa, synthetic construct
  REGION 1..19: note = synthetic
MGWSCIILFLVATATGVHS

SEQ ID NO: 77 - protein, 448 aa, synthetic construct
  REGION 1..448: note = synthetic
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 78 - protein, 214 aa, synthetic construct
  REGION 1..214: note = synthetic
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - protein, 307 aa, synthetic construct
  REGION 1..307: note = synthetic
MALPVTALLLPLALLLHAARPMFAMKTKAALAIWCPGYSETQINATQAMKKARKSKVTTNKCLEQVSQLQGLWRRFNRPLLKQQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 375 aa, synthetic construct
  REGION 1..375: note = synthetic
MALPVTALLLPLALLLHAARPYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKARKSKVTTNKCLEQVSQLQGLWRRFNRPLLKQQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 375 aa, synthetic construct
  REGION 1..375: note = synthetic
MALPVTALLLPLALLLHAARPYDFTNCDFQKIEADYLRTISKDLITYMSGTKSTDFNNTVSCSNRPHCLTEIQSLTFNPTPRCASLAKEMFARKTKATLALWCPGYSETQINATQAMKKRRKRKVTTNKCLEQVSQLLGLWRRFIRTLLKKQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 131 aa, synthetic construct
  REGION 1..131: note = synthetic
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 83 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 84 - protein, 107 aa, synthetic construct [sequence omitted]

SEQ ID NO: 85 - protein, 159 aa, synthetic construct
  REGION 1..159: note = synthetic
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 86 - protein, 122 aa, synthetic construct
  REGION 1..122: note = synthetic
QVQLVQSGAEVKKPGASVKVSCKSSGYAFSSYWMNWVRQAPGQGLEWMGQIFPGDGDTTYNGNFKGRVTMTRDTSTSTVYMELSSLRSEDTAVYYCARSGFDYGYFYAMDYWGQGTTVTVSS

SEQ ID NO: 87 - protein, 25 aa, synthetic construct
  REGION 1..25: note = synthetic
QVQLVQSGAEVKKPGASVKVSCKSS

SEQ ID NO: 88 - protein, 330 aa, synthetic construct
  REGION 1..330: note = synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK